(19) United States
(12) Patent Application Publication
(10) Pub. No.: US 2015/0289861 A1
(43) Pub. Date: Oct. 15, 2015

(54) DEVICES FOR USE WITH HEMOSTATIC MATERIALS

(71) Applicant: STB, Ltd., Bellevue, WA (US)

(72) Inventors: Martin MacPhee, Darnestown, MD (US); Dawson Beall, Germantown, MD (US); Jennifer Brichetti, Gaithersburg, MD (US); Belinda Wilmer, Martinsburg, WV (US)

(21) Appl. No.: 14/656,685

(22) Filed: Mar. 12, 2015

Related U.S. Application Data
(60) Provisional application No. 61/951,956, filed on Mar. 12, 2014.

(51) Int. Cl. A61B 17/00; A61M 35/00

(52) U.S. Cl. CPC A61B 17/0057; A61B 17/00234; A61M 35/00

(57) ABSTRACT

Disclosed are solid and frozen haemostatic materials and dressings consisting essentially of a fibrinogen component and a fibrinogen activator. Also disclosed are methods of treating internal wounded tissue in a mammal by applying one or more of these haemostatic materials and dressings, particularly for the treatment of injured tissue via endoscopic or minimally-invasive surgical techniques.

### FIELD OF THE INVENTION

The present invention relates to compositions and applicators for treating injured tissue in a mammalian patient, such as a human and methods of using the same.

### BACKGROUND OF THE INVENTION

There are a large number of medical procedures that result in injuries to blood vessels. Similarly, there are numerous examples of bleeding caused by traumatic injuries, hematological disorders, and from unknown causes. When the site of bleeding is not readily accessible, such as an injured vessel located deep within the flesh, or inside a body cavity, a simple and effective method of hemorrhage control that can access the site within the body and seal the injured vessel is needed. Similarly, tissue may be divided by either traumatic injury or surgical procedure, and require sealing to approximate the edges of the injury in order to restore function. The same problems may also occur in regards to open wounds, or damaged tissue inside the respiratory, alimentary, reproductive, urinary, auditory and digestive tracts as well as other tissue tracts that communicate with the outside of the body such as tear ducts. Current sealing products and devices have one or more deficiencies, usually due to their inadequate performance, or their reliance upon non-natural components that interfere with normal healing, or fundamental difficulties with conveniently and effectively applying them.

The need for improved technologies to address these injuries is significant. For example, in the case of blood vessels that have been deliberately punctured as part of a diagnostic and/or therapeutic procedure (such as cardiac catheterization, balloon angioplasty, vascular stenting and the like), over seven million such procedures are currently performed every year, but with a 9% overall complication rate and a 1-3% major complication rate (See Millennium Research Group: Global Markets for Vascular Closure Devices 2006). These complications can lead to significant morbidity, increased expense, a requirement for additional procedures and/or devices, extended time in the medical facility and conversion of outpatients to inpatients. Commercially available products now available only reduce the major complication rate by one half of one percent (See Arora et al: Am Heart J. 2007 April; 153(4):606-11) to 2.4%, Nevertheless, despite this poor performance, even these devices are currently used since the costs and consequences of procedure-induced complications are so high (See Resnic et al: Am J Cardiol. 2007 Mar. 15; 99(6):766-70).

Not only are there the above described complications associated with therapy itself, closure of the access hole(s) created in the blood vessel is a significant source of additional complications, including uncontrolled hemorrhage, pseudoaneurysm, hematoma, arteriovenous fistula, arterial thrombosis, infection, and retained devices (See Meyerson et al.: Angiographic Access Site Complications in the Era of Arterial Closure Devices Vasc Endovasc Surg. 2002; 36 (2) 137-44). These additional complications may lead to prolonged closure procedures, hospitalization, the requirement for surgical repair, and even tissue loss or death.

Currently, the primary means of closing the access hole in the vessel has been to allow a natural blood clot to form at the puncture site. This has generally been accomplished by manual compression, but various products have recently been developed in an attempt to reduce the time required to achieve vascular closure. Such devices automate the application of pressure over the injury site, suture the hole in the vessel, clip the hole shut, or apply some sort of patch or pad that allegedly increases the formation of a natural clot at the site. These devices are convenient and gaining in popularity, but their overall safety appears over estimated. Indeed, far from being risk free, these devices may be associated with unique levels of hemorrhagic and cardiac risks including myocardial infarction, stroke and death (See Rao, S. Implication of bleeding and blood transfusion in percutaneous coronary intervention. Rev Cardiovasc Med. 2007, 8 Suppl 3:S18-26.).

Significant risks have been reported to be associated with ail classes of vascular closure devices. Most seriously, the severity and the difficulty is treating complications are generally greater when vascular closure devices are used (See Nehler et al. Iatrogenic vascular injuries from percutaneous vascular suturing devices. J. Vasc Surg 2001 May; 33(5):943-7; Castelli et al: Incidences of vascular injuries after use of the Angio-Seal closure device following endovascular procedures in a single center. World J Surg. 2006 March; 30(3):280-4.). The use of such devices is even associated with higher risks among patients having complications of pseudoaneurysms, failure to successfully test such pseudoaneurysms, blood loss, transfusions, extensive operations to correct the problems and arterial infections (See Sprouse et al. The management of peripheral vascular complications associated with the use of percutaneous suture-mediated closure devices. J Vasc Surg. 2001. April; 33(4):688-693.). Moreover, some of these complications can be deadly, particularly in patients with diabetes, obesity and previously implanted devices (all conditions commonly found in patients in whom such closure devices are frequently used) (See Hollis and Rehring. Femoral endarteritis associated with percutaneous suture closure: new technology, challenging complications. J Vasc Surg. 2003 July; 38(1):83-7.). Accordingly, there remains a great need to develop a vascular closure system that avoids the problems associated with use of knows vascular closure devices.

Another medical situation involving treatment of injured internal tissue is the repair of herniations. There are numerous types and locations of hernia, and the surgical repair techniques vary widely depending thereon. Both open and endoscopic procedures are currently in use, and may involve the use of sutures alone or sutures in combination with various kinds of meshes or supports for the injured tissue. Major complications for most hernia repair procedures include pain and the requirement to re-do the repair (See American College of Surgeons. When you need an operation . . . About Hernia Repair, available at: http://www/facs.org/public_info/operation/hernrep.pdf).

Similarly, there is also a need to improve the therapeutic options for treatment of simple bleeding conditions such as epistaxis, which requires professional medical treatment in 1 of 7 people in their lifetime (See Evans: Epistaxis. emedicine (2007) available at www.emedicine.com/EMERG/topic806.htm). In fact, epistaxis is frequently cited as the most common ENT emergency (See Hussain et al: Evalutaion of aetology and efficacy of management protocols of epistaxis. Ayub Med Coll Abottabad, 2006 October-December; 18(4):63-6). The difficulty in treating these cases is evidenced by the face that 1.6 out of every 10,000 patients are hospitalized for epistaxis that is refractory to normal treatment (See Viehweg et al: Epistaxis: diagnosis and treatment. J. Oral Maxillofac Surg 2006 March; 64(3):5 11-8). Current treatment options include packing, chemical cauterization, electrocautery, surgical ligation and embolization (See: Ortiz & Bhattacharyya: Managemnt pittfalls in the use of embolization for the treatment of severe epistaxis, Ear Nose Throat J. 2002 March; 82(3): 178-83.) Frequently, multiple treatments with different technologies are required to effectively treat this often life-threatening condition (See Siniluoto et al: Embolization for the treatment of posterior epistaxis. An analysis of 31 cases. Arch Otolaryngol Head Neck Surg. 1993 August; 119(8):837-41; Gifford & Orlandi: Epistaxis. Otoloaryngol Clin North Am. 2008 June; 41(3)S25-36, vii).

There are now in use a number of newer haemostatic agents that have bees developed to overcome the deficiencies of traditional gauze bandages. These haemostatic agents include the following:
Microporous polysaccharide particles (TraumaDEX®, Medafor Inc., Minneapolis, Minn.);
Zeolite (QuikClot®, Z-Medica Corp, Wallington, Conn.);
Acetylated poly-N-acetyl glucosamine (Rapid Deployment Hemostat™ (RDH), Marine Polymer Technologies, Danvers, Mass.);
Chitosan (HemCon® bandage, HemCon Medical Technologies Inc. Portland Oreg.);
Liquid Fibrin Sealants (Tiseel VH, Baxter, Dearfield, Ill.)
Human fibrinogen and thrombin on equine collagen (TachoComb-S, Hafslund Nycomed Pharma, Linz, Austria);
Microdispersed oxidized cellulose (m*doc™, Alltracel Group, Dublin, Ireland);
Propyl gallate (Hemostatin™, Analytical Control Systems Inc., Fishers, Ind.);
Epsilon aminocaproic acid and thrombin (Hemarrest™ patch, Clarion Pharmaceuticals, Inc);
Purified bovine corium collagen (Avitene® sheets (non-woven web or Avitene Microfibrillar Collagen Hemostat (MCH), Davol, Inc., Cranston, R.I.);
Controlled oxidation of regenerated cellulose (Surgicel®, Ethicon Inc., Somerville, N.J.);
Aluminum sulfate with an ethyl cellulose coating (Sorbastace Microcaps, Hemostace, LLC, New Orleans, La.);
Microporous hydrogen-forming polyacrylamide (BioHemostat, Hemodyne, Inc., Richmond Va.); and
Recombinant activated factor VII (NovoSeven®, NovoNordisk Inc., Princeton, N.J.).
   These agents have met with varying degrees of success when used in animal models of traumatic injuries and/or in the field, and with limited success in the sealing of therapeutic vascular injuries.

Liquid fibrin sealants, such as Tissel VH, have been used for years as an operating room adjunct for hemorrhage control. See J. L., Garza et al., J. Trauma 30:512-513 (1990); H. B. Kram et al., J. Trauma 30:97-101(1990); M. G. Ochsner et al., J. Trauma 30:884-887 (1990); T. L., Matthew et al., Ann. Thorac. Surg. 50:40-44 (1990); H. Jakob et al., J. Vasc. Surg., 1:171-180 (1984). The first mention of tissue glue used for hemostasis dates back to 1909. See Current Trends in Surgical Tissue Adhesives: Proceedings of the First International Symposium on Surgical Adhesives, M. J. MacPhee et al., eds. (Lancaster, Pa.: Technomic Publishing Co; 1995). Liquid fibrin sealants are typically composed of fibrinogen and thrombin, but may also contain Factor XIII/XIIIa, either as a by-product of fibrinogen purification or as an added ingredient (in certain applications, it is therefore not necessary that Factor XIII/Factor XIIIa be present in the fibrin sealant because there is sufficient Factor XIII/XIIIa, or other transaminase, endogenously present to induce fibrin formation). As liquids, however these fibrin sealants have not proved useful outside certain specific procedures.

Dry fibrinogen-thrombin dressings having a collagen support (e.g. TachoComb™, TachoComb™ H and TachoSil available from Hafslund Nycomed Pharma, Linz, Austria) are also available for operating room use is many European countries. See U. Schiele et al., Clin. Materials 9:169-177 (1992). While these fibrinogen-thrombin dressings do not require the pre-mixing needed by liquid fibrin sealants, their utility for field applications is limited by a requirement for storage at 4° C. and the necessity for pre-wetting with saline solution prior to application to the wound. These dressings are also not effective against high pressure, high volume bleeding. See Sondeen et al., J. Trauma 54:280-285 (2003).

A dry fibrinogen/thrombin dressing for treating wounded tissue is also disclosed in U.S. Pat. No. 8,762,336. This particular dressing is composed of a backing material and a plurality of layers, the outer two of which contain fibrinogen (but no thrombin) while the inner layer contains thrombin and calcium chloride (but no fibrinogen). While this dressing has shown great success in several animal models of hemorrhage, the bandage is fragile, inflexible, and has a tendency to break apart when handled. See McManus et al. Business Briefing: Emergency Medical Review 2005, at 78.; Kheirabadi et al., J. Trauma 59:25-35 (2005). In addition, U.S. Pat. No. 6,762,336 teaches that this bandage should contain 15 mg/cm² of fibrinogen to successfully pass a porcine arteriotomy test that is less robust than that disclosed in this application (see Example XI). Moreover, although U.S. Pat. No. 6,762,336 discloses that bandages comprising two layers of fibrinogen, each with a concentration of 4 mg/cm² to 15 mg/cm² may provide effective control of hemorrhage, it further teaches that “fibrinogen dose is related to quality. The higher dose is associated with more firm and tightly adhered clots. While lower fibrinogen doses are effective for hemorrhage control daring the initial 60 minutes, longer term survival will likely depend on clot quality.”

Other fibrinogen/thrombin-based dressings have also been proposed. For example, U.S. Pat. No. 4,683,142 discloses a resorptive sheet material for closing and healing wounds which consists of a glycoprotein matrix, such as collagen, containing coagulation proteins, such as fibrinogen and thrombin. U.S. Pat. No. 5,702,715 discloses a reinforced biological sealant composed of separate layers of fibrinogen and thrombin, at least one of which also contains a reinforcement filler such as PEG, PVP, BSA, mannitol, FICOLL, dextran, myo-inositol or sodium chlorate. U.S. Pat. No. 6,056,970 discloses dressings composed of a bioabsorbable polymer, such as hyaluronic acid or carboxymethylcellulose, and a haemostatic composition composed of powdered thrombin and/or powdered fibrinogen. U.S. Pat. No. 7,189,410 discloses a bandage composed of a hacking material having thereon: (i) particles of fibrinogen; (ii) particles of thrombin; and (iii) calcium chloride. U.S. Patent Application Publication No. US 2006/0155234 A1 discloses a dressing composed of a backing material and a plurality of fibrinogen layers which have discrete areas of thrombin between them. To date, none of these dressings have been approved for use or are available commercially.

Minimally invasive procedures often have strict requirements for attaining hemostasis. For the most part, the body cavities being treated are reached by either natural orifices or by small holes, and thus the instruments that can reach the treatment sites are themselves of a small diameter. This limits their complexity and dexterity, with a resulting limit on the general effectiveness of hemostatic products that can be used. The primary tools include direct pressure, sometime supplemented with a small amount of gauze at the tissue-instrument interface, and cautery. Should these tools fail, the only option is to convert the ‘closed’ minimally-invasive surgical procedure to a traditional ‘open’ one, with the attendant disadvantages of increased risk to the Patient, increased Patient morbidity, increased surgical time and increased costs. Thus any invention that improves the chances of achieving hemostasis during a minimally invasive procedure is highly desirable. Furthermore, current limitations on the ability to achieve hemostasis using the available endoscopic products limits the number of operations that can be initiated as endoscopic procedures, placing a further value on more capable endoscopic hemorrhage control technologies.

The same is true for procedures that involve treating wounds, whether medical or traumatic, that involve wound ‘tracts’ that lead form the exterior surface of the body deep into tissue. Current technologies for treating these situations are few and minimally effective, and further improvements highly desirable, it is even the case that open wounds or open surgical procedures may benefit from treatment with more effective, convenient, ready to use and economical hemostatic technologies.

A number of different techniques, including the use of liquid fibrin sealant, have been proposed for sealing punctures in blood vessels, including those made to secure vascular access. For example, U.S. Pat. No. 7,357,794 discloses devices, systems and methods for acute or chronic delivery of substances or apparatus to extravascular treatment sites. U.S. Pat. No. 7,335,220 discloses apparatus and methods for sealing a vascular puncture using an expanding lyophylized hydrogel plug. U.S. Pat. No. 7,300,663 discloses adhesion and sealing of tissue with compositions containing polyfunctional crosslinking agents and protein polymers. U.S. Pat. No. 7,399,483 discloses a carrier with solid fibrinogen and solid thrombin; U.S. Pat. No. 7,335,220 discloses apparatus and methods for sealing vascular punctures. U.S. Pat. No. 7,115,588 discloses methods for treating a breach or puncture in a blood vessel. U.S. Pat. No. 7,008,442 discloses vascular sealant delivery devices using liquid formulations. U.S. Pat. No. 6,890,342 discloses to methods and apparatus for closing vascular puncture using a guidewire and/or other surgical implement extending from the wound on which a haemostatic material is moved into contact with an area of the blood vessel surrounding the wound. U.S. Pat. No. 6,818,088 discloses percutaneous puncture sealing method using flowable sealants. U.S. Pat. No. 6,699,262 discloses a percutaneous tissue track closure assembly and method using flowable materials. U.S. Pat. No. 6,613,070 discloses sealing vascular penetrations with haemostatic gels. U.S. Pat. No. 6,500,152 discloses a device for introducing a two-component liquid fibrin adhesive into a puncture channel. U.S. Pat. No. 6,325,789 also discloses a device for sealing puncture wounds using liquid or paste fibrin sealant. U.S. Pat. No. 5,814,066 discloses methods of reducing femoral arterial bleeding using percutaneous application of liquid fibrin sealant. U.S. Pat. No. 5,725,551, U.S. Pat. No. 5,486,195 and U.S. Pat. No. 5,443,481 each disclose the use of two component liquid fibrin sealant for artery closure. U.S. Pat. No. 5,649,959 discloses an assembly for sealing a puncture is a vessel which maintains the fibrinogen and thrombin separately. To date, however, all of these remain little-used in therapy, most likely doe to the difficult and time consuming preparation requirements for two-component liquid fibrin sealant compositions.

Liquid fibrin sealant has also been used to treat epistaxis, endoscopic sinus surgery and endonasal surgery ((See Vaiman et al. Fibrin glue treatment for epistaxis. Rhinology. 2002 June; 40(2):99-91; Vaiman et al. Use of fibrin glue as a haemostatic in endoscopic sinus surgery. Ann Otol Rhinol Laryngol, 2005 March; 114(3): 237-41; Vaiman et al. Fibrin sealant: alternative to nasal packing in endonasal operations. A prospective randomized study. Isr Med Assoc J. 2005 September; 7(9):571-4.). All these reports indicate that liquid fibrin sealant may he used with some success at controlling hemorrhage from various locations just inside the nose all the way into the sinuses. However, the time and efforts associated with preparing such sealants make them less than ideal for daily clinical use. Their effectiveness may be further limited by the difficulties in combining their application with direct pressure during the period required for fibrin formation.

Accordingly, there remains a need in the art for compositions of solid hemostatic materials and effective, convenient means of applying them to achieve hemostasis and sealing of both internal and external wounded tissue, particularly highly vascularized tissue, and single blood vessels. Additionally, treatment of tissues that have been divided (e.g. due to accident, pathology or surgical intervention) and require re-approximation to promote healing would also benefit from such materials and applicators capable of adequate tissue sealing.

The assessment of such materials requires new techniques that go beyond those previously disclosed for testing haemostatic dressings. The ability of dressings to seal an injured blood vessel has been determined by an ex vivo porcine arteriotomy (EVPA) performance test, which was first described in U.S. Pat. No. 6,762,336. The EVPA performance test evaluates the ability of a dressing to stop fluid flow through a hole in a porcine artery. While the procedure described in U.S. Pat. No. 6,762,336 has been shown to be useful for evaluating haemostatic dressings, it failed to replicate faithfully the requirements for success in vivo. More specifically, the procedure disclosed in U.S. Pat. No. 6,762,336 required testing at 37° C., whereas, in the real world, wounds are typically cooler than that this decreased temperature can significantly reduce the rate of fibrin formation and its haemostatic efficacy in trauma victims. See, e.g., Acheson et al., J. Trauma 59:865-874 (2005). The test in U.S. Pat. No. 6,762,336 also failed to require a high degree of adherence of the dressing to the injured tissue. A failure mode in which fibrin forms but the dressing fails to attach tightly to the tissue would, therefore, not be detected by this test. Additionally, the pressure utilized in the procedure (200 mHg) may be exceeded during therapy for some trauma patients. The overall result of this is that numerous animal tests, typically involving small animals (such as rats and rabbits), must be conducted to accurately predict dressing performance in large animal, realistic trauma studies and in the clinical environment.

In order to minimize the amount of time and the number of animal studies required to develop dressings intended to treat accessible traumatic injuries, an improved ex vivo testing procedure has been developed. To accomplish this, the basic conditions under which the dressing test was conducted were changed, and the severity of the test parameters was increased to include testing at lower temperatures (i.e. 29-33° C. vs. 37° C., representing the real physiologic challenge at realistic wound temperatures (Acheson et al., J. Trauma 59:865-874 (2005)), higher pressures (i.e. 250 mmHg vs. 200 mmHg), a longer test period (3 minutes vs. 2 minutes) and larger sized arterial injuries (U.S. Pat. No. 6,762,336 used an 18 gauge needle puncture, whereas the revised procedure used puncture holes ranging from 2.8 mm to 4 mm×6 mm). A new test has also been developed to directly measure adherence of the dressing to the injured tissue. Both these tests showed greatly improved stringency and are thus capable of surpassing the previous ex vivo test and replacing many in vivo tests for efficacy. These newer tests are described in U.S. patent application Ser. No. 11/882,874, the disclosure of which is herein incorporated by reference in its entirety.

The newer tests described in U.S. patent application Ser. No. 11/882,874 were designed to simulate trauma-derived, accessible wounds with high pressure and flow characteristics. Therefore, for the evaluation of methods and compositions for treating wounded internal tissue, it was preferable to develop additional assays to more accurately simulate the peripheral vasculature and the effects of surrounding tissue.

### SUMMARY OF THE INVENTION

It is therefore an object of the present invention to provide solid dressings that can treat wounded internal mammalian tissue. It is further an object of the present invention to provide a method of treating wounded Internal mammalian tissue, particularly human tissue. Other objects, features and advantages of the present invention will be set forth in the detailed description of preferred embodiments that follows, and will in part be apparent from that description and/or may be learned by practice of the present invention. These objects and advantages will be realized and attained by the compositions and methods described in this specification and particularly pointed out in the claims that follow.

In accordance with these and other objects, a first embodiment of the present invention is directed to a method for treating wounded internal tissue in a mammal comprising applying to wounded internal tissue a haemostatic putty material consisting essentially of a fibrinogen component and a fibrinogen activator that can be applied with an applicator or by hand to treat wounded internal tissue.

Another embodiment is directed to a method for treating wounded internal tissue in a mammal comprising applying to wounded internal tissue at least one haemostatic material consisting essentially of a fibrinogen component and a fibrinogen activator that is ground up into a powder and then reformed into pellets, in combination with other excipients to create a haemostatic material suitable for treatment of wounded tissue.

Another embodiment is directed to a method for treating wounded internal tissue in a mammal comprising applying to a powder consisting essentially of a fibrinogen component and a fibrinogen activator that is compressed into a suitable shape and attachable to an applicator for treatment of wounded tissue.

Additional embodiments are directed to the design of applicators suitable for use of the hemostatic materials and facilitating their use on various tissues, whether accessing the site to be treated via a conventional ‘open’ surgical technique or by as endoscopic, minimally invasive-type approach. When the product format is for endoscopic use an applicator may have one or more of several kinds of features designed to hold the product firmly to the applicator tip and allow it to be pressed onto the site to be treated until such time as the application has complete and to then release the product from the applicator. This may be achieved by the use of clamps, quills, hook and loop fasteners, or a suitable break-away layer, or the product may be affixed by some kind of thread that can be withdrawn so as to no longer hold it to the applicator when desired.

Another embodiment would include an applicator that is rod-like in shape, which may have one or more of the following additional features: a handle, a trigger-release to free the product from the end.

Another embodiment would include a provision, preferably co-axial to the applicator shaft(s), to provide for application of a suitable fluid to the application site or product to facilitate its application, dissolution or adherence to the tissue and or release from the applicator.

Another embodiment would include a provision, preferably co-axial to the applicator shaft(s), to provide for application of a mild vacuum or suction to remove blood or other bodily fluids, irrigation fluid or residual product components from the application site before, during or after application.

Still another embodiment would include a provision, preferably co-axial to the applicator shaft(s), to provide for application of multiple products to multiple sites using a single applicator, either simultaneously or sequentially.

Another embodiment would include a provision, preferably co-axial to the applicator shaft(s), to provide for application of illumination of a frequency that causes blood or other bodily fields to be easily identified, via visual inspection, either via optics incorporated into the applicator itself or into another device inserted into the body cavity to be treated.

Another embodiment would include a provision, preferably co-axial to the applicator shaft(s), to provide for application of a controlled or limited amount of application pressure to facilitate sale and effective treatment, particularly for delicate tissues. In one embodiment this could consist of a pressure monitoring device that alerts the operator to excessive application pressure. An additional embodiment would be a device that alerts the operator that sufficient pressure is being applied, and further it may also alert to the application of excessive pressure in a fashion distinct from the alert for sufficient pressure, thus allowing the operator to easily maintain pressure in the optimal range throughout the application. Suitable alerts would include, but not be limited to, auditory, visual and haptic means.

Another embodiment would include a provision, preferably co-axial to the applicator shaft(s), to provide for an timer alert to tell the operator when pressure has been applied for a sufficient period of time. This may operate automatically when sufficient pressure is applied by the Operator or at the Operators instigation. Suitable alerts would include, but not be limited to, auditory, visual and haptic means.

It is to be understood that the foregoing general description and the following detailed description of preferred embodiments are exemplary and explanatory only and are intended to provide further explanation, but not limitation, of the invention as claimed herein.

### BRIEF DESCRIPTION OF DRAWINGS

FIG. 1 is a diagram of the set-up for the ex vivo porcine carotid arteriotomoy assay described herein.

FIGS. 2A, 2B, and 2C depict an embodiment having a plunger, a tube and a absorbable material disposed of on said plunger so as to absorb some or all of the haemostatic liquid material bandage, before pressing the material to a wound site.

FIGS. 3A, 3B and 3C depict an alternative embodiment as described herein having a backing and an absorbent material.

FIGS. 4A-4E depict an applicator used in connection with a sheath and a haemostatic material.

FIGS. 5A-5F depict various embodiments of means to connect a plunger or applicator to a haemostatic material or backing.

FIGS. 6A, 6B, and 6C depict embodiments of an applicator with a haemostatic material secured thereto.

FIGS. 7A, 7B, and 7C depict embodiments utilizing an absorbable material at the end of an applicator.

FIG. 8 depicts a SDS-PAGE Gel of a haemostatic dressing described by an embodiment described herein.

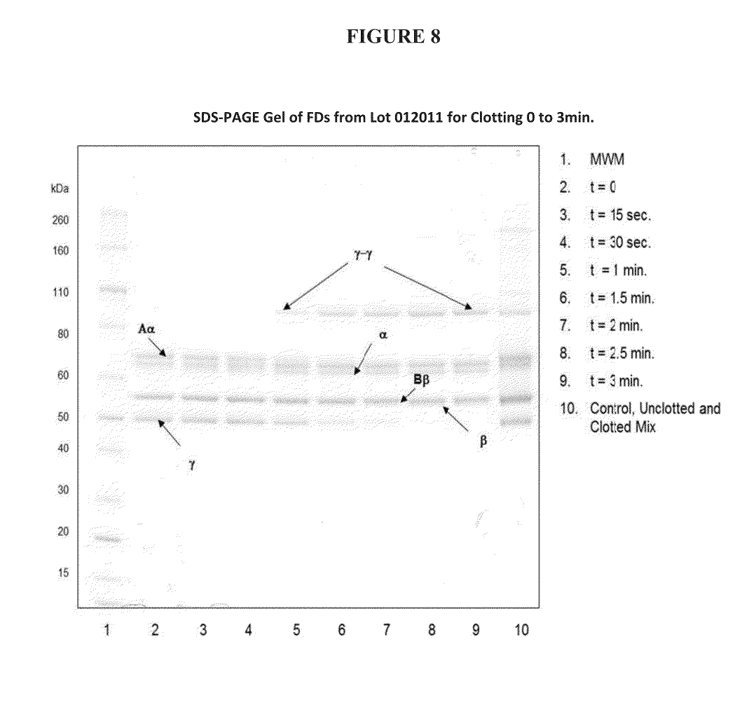

FIG. 9 depicts a Gel showing the clotting cascade from 3.5 to 10 minutes of a haemostatic dressing described by an embodiment described herein.

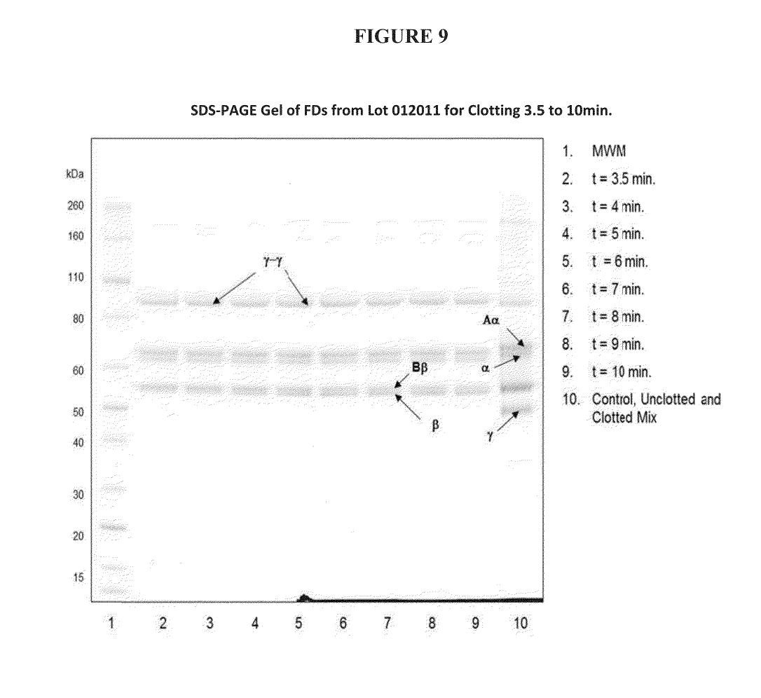

FIG. 10 is a graphical depiction of the clotting of a haemostatic dressing described by an embodiment described herein.

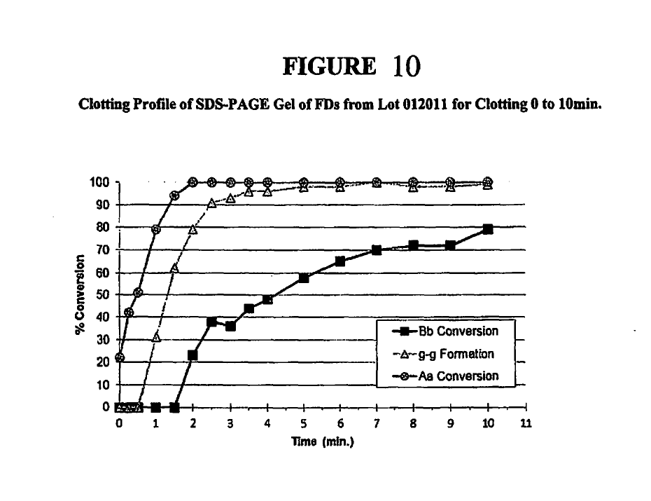

FIGS. 11A-11F depict various embodiments of a rod shaped haemostatic material, a plunger, and attachment mechanisms to said plunger.

FIGS. 12A-12E depict embodiments utilizing a sheath to form a cylindrical haemostatic material.

### DETAILED DESCRIPTION OF PREFERRED EMBODIMENTS

Unless defined otherwise, all technical and scientific terms used herein have the same meaning as is commonly understood by one of skill in the art to which this invention belongs.

All patents and publications mentioned herein are incorporated by reference.

As used herein, use of a singular article such as “a,” “an,” and “the” is not intended to excluded pluralities of the article's object unless the context clearly and unambiguously dictates otherwise.

“Patient” as used herein refers to human or animal individuals in need of medical care and/or treatment.

“Wound” or “wounded tissue” as used herein refers to any damage to any internal tissue of a patient which results in the loss of blood from the circulatory system and/or any other fluid from the patient's body. The tissue may be any mammalian internal tissue, such as an organ or blood vessel. A wound may be in a soft internal tissue, such as an organ, or in hard internal tissue, such as bone. The “damage” may have been caused by any agent or source, including traumatic injury, infection or surgical intervention. Thus, the “damage” being treated according to the methods of the present invention may be the result of either an accident or an intentional act.

“Resorbable material” as used herein refers to a substance that is broken down spontaneously and/or by the mammalian body into components which are consumed or eliminated in such a manner as not to interfere significantly with wound healing and/or tissue regeneration, and without causing any significant metabolic disturbance.

“Stability” as used herein refers to the retention of those characteristics of a substance that determine activity and/or function.

“Suitable” as used herein is intended to mean that a substance (or mixture of substances) does not adversely affect the stability of the dressings or any component thereof.

“Binding agent” as used herein refers to a compound or mixture of compounds that improves the adherence and/or cohesion of the components of the haemostatic material of the dressings.

“Solubilizing agent” as used herein refers to a compound or mixture of compounds that improves the dissolution of a protein or proteins in aqueous solvent.

“Filler” as used herein refers to a compound or mixture of compounds that provide bulk and/or porosity to the haemostatic material.

“Release agent” as used herein refers to a compound or mixture of compounds that facilitates removal of a dressing from a manufacturing mold.

“Foaming agent” as used herein refers to a compound or mixture of compounds that produces gas when hydrated under suitable conditions.

“Solid” as used herein is intended to mean that a haemostatic material or dressing will not substantially change in shape or form when placed on a rigid surface and then left to stand at mom temperature for 24 hours.

“Frozen” as used herein is intended to mean that a haemostatic material or dressing will not substantially change In shape or form when placed on a rigid surface and then left to stand at 0° C. for 24 hours, but will substantially change in shape or form when placed on a rigid surface and then left at room temperature for 24 hours. Thus, in the context of the present invention, a “solid” dressing is not “frozen” and a “frozen” composition is not “solid”.

“Substantially homogeneous” is used herein is intended to mean that the haemostatic material has a uniform composition throughout, within the tolerances described herein. Thus, a “substantially homogeneous” haemostatic material according to the present invention may be composed of a plurality of particles, provided that each of those particles has the same composition.

“γ-γ dimer” as used herein, means covalently cross-linked fibrinogen γ chains. Since the resulting structure has a higher apparent molecular weight then single γ chains, and can be separated from the α and β chains by molecular weight, the relative amount of γ-γ versus free γ chains in a sample can be determined. Further, since the formation of γ-γ dimers from γ chains occurs late in the transformation of fibrinogen to insoluble fibrin. It can be used to quantify the amount of fibrin in a sample.

As used herein, “fibrin” refers to fibrin polymers, predominantly cross-linked via their gamma chains that are substantially insoluble under physiological conditions.

“About” as used herein means within 10% of a stated number.

“Substantially” as used herein means something done with the intent of the action being complete but allowing for 5% variance. For example substantially unreacted, means intended to have no reaction, but allowing up to 5% reaction to have occurred.

As used herein, “consisting essentially of” is intended to mean that the fibrinogen component and the fibrinogen activator are the only necessary and essential ingredients of the haemostatic material when it is used as intended to treat wounded internal tissue. Accordingly, the haemostatic material may contain other ingredients in addition to the fibrinogen component and the fibrinogen activator as desired for a particular application, but these other ingredients are not required for the solid dressing to function as intended under normal conditions, i.e. these other ingredients are not necessary for the fibrinogen component and fibrinogen activator to react and form enough fibrin to reduce the flow of blood and/or fluid from normal wounded tissue when that dressing is applied to that tissue under the intended conditions of use. If, however, the conditions of use in a particular situation are not normal, for example the patient is a hemophiliac suffering from Factor XIII deficiency, then the appropriate additional components, such as Factor XIII/XIIIa or some other transaminase, may be added to the haemostatic material without deviating from the spirit of the present invention.

According to certain embodiments of the present invention, the haemostatic material is formed or cast as a putty. The particular nature of the formation of such a material requires certain conditional and drying regimens to form the putty without forming a fibrin based material.

According to certain embodiments, the ability to take a preformed FD dressing, and grind it into a powder and then compress it into certain shapes. See example 10 and 12.

According to other preferred embodiments, it is advantageous to utilize methods and products as instructed in Example 25.

Once such is formed or cast, the haemostatic material may then be used as is or it may be former processed, for example by grinding into a powder of predetermined particle size. Such particles may then be used as is or may be combined with other substances for a particular application, e.g. such particles of haemostatic material may be mixed with a foaming agent or aerosol gas or may be combined with one or more binding agents and applied to a support material.

The haemostatic materials of the present invention may be formed or cast in any shape or form suitable for a given application. For example, the haemostatic material may be formed or cast in the shape of a cone or cylinder or the like. Such a shape is particularly suitable for use in applications where the damage to the tissue being treated is a hole to be plugged or sealed, e.g a vein which has been intentionally punctured as part of a medical procedure, such as angioplasty. In such applications, the haemostatic material may alternatively be in the shape of a disk, optionally with a hole for use in conjunction with a guide wire. Additionally, each of these forms can also be prepared by combining particles of the inventive haemostatic materials with at least one suitable binding agent in an appropriate mold.

The haemostatic material may also he formed or cast in the shape of a flat sheet. Such a form is particularly suitable for use in applications where tissue needs to be sealed, or approximated, for example in connection with endoscopic surgery or, hernia repair. Alternatively, a flat sheet may be prepared by combining particles of the inventive haemostatic materials with one or more suitable binding agents, optionally in a mold.

In suitable situations, a haemostatic material may be formed in a mold to conform to a specified shape for use in a specified type of setting. Shapes may include circular, oval, square, or other shapes as necessary. Furthermore, the haemostatic material may comprise a length, a width, and a depth, such that a three-dimensional haemostatic material may properly seal a wound. A wound may comprise a particular surgical opening or close a fistula, or for other need to seal a wound.

Typical situations may arise when an endoscope makes an incision and requires a haemostatic material of a particular shape and size for a particular surgery. Such a haemostaic material may be pre-formed to that suitable surgery. Such haemostatic material may comprise a backing or no backing, and may comprise a release mechanism or none.

Suitable hemostatic materials may comprise thrombin alone, fibrinogen alone, fibrinogen activators alone, fibrinogen components alone, or combinations thereof. Furthermore, anti-clotting materials may be incorporated into a haemostatic material, either alone or in combination with any fibrinogen activator or fibrinogen component.

Suitable ranges and optimization of the composition of the material may comprise about 0.1 mg/cm² to about 15.0 mg/cm² fibrinogen component and comprise about 0.01 U/mg to about 10 U/mg fibrinogen of a fibrinogen activator.

The haemostatic material may also optionally contain one or more suitable fillers, such as sucrose, lactose, maltose, silk, fibrin, collagen, albumin (natural or recombinantly produced), polysorbate (Tween™), chitin, chitosan and its derivatives (e.g. NOCC-chitosan), alginic acid and salts thereof, cellulose and derivatives thereof, proteoglycans, hyaluron and its derivatives, such as hyaluronic acid, glycolic acid-polymers, lactic acid polymers, glycolic acid/lactic acid co-polymers, and mixtures of two or more thereof.

The haemostatic material may also optionally contain one or more suitable solubilizing agents, including detergents and tensides. Illustrative examples of suitable solubilizing agents include, but are not limited to, the following: sucrose, dextrose, mannose, trehalose, mannitol, sorbitol, albumin, hyaluron and its derivatives, such as hyaluronic acid, sorbate, polysorbate (Tween™), sorbitan (SPAN™) and mixtures of two or more thereof.

The haemostatic material may also optionally contain one or more suitable foaming agents, such as a mixture of a physiologically acceptable acid citric acid or acetic acid) and a physiologically suitable base (e.g. sodium bicarbonate or calcium carbonate). Other suitable foaming agents-include, but are not limited to, dry particles containing pressurized gas, such as sugar particles containing carbon dioxide (see, e.g., U.S. Pat. No. 3,012,893) or other physiologically acceptable gases (e.g. Nitrogen or Argon), and pharmacologically acceptable peroxides. Such a foaming agent may be introduced into the aqueous mixture of the fibrinogen component and the fibrinogen activator, or may be introduced into an aqueous solution of the fibrinogen component and/or an aqueous solution of the fibrinogen activator prior to mixing. Alternatively, the inventive haemostatic materials may be ground to particles of a predetermined size and then combined with a suitable foaming agent.

The haemostatic material may also optionally contain a suitable source of calcium ions, such as calcium chloride, and/or a fibrin cross-linker, such as a transaminase (e.g. Factor XIII/XIIIa) or glutaraldehyde.

The haemostatic materials of the present invention are most preferably prepared by mixing aqueous solutions of the fibrinogen component and the fibrinogen activator under conditions which minimize the activation of the fibrinogen component by the fibrinogen activator. This aqueous mixture of the fibrinogen component and the fibrinogen activator may then be frozen until used to treat wounded tissue. Alternatively, the mixture may then subjected to a process, such as lyophilization or freeze-drying, to reduce the moisture content to a predetermined effective level, to a level where the dressing is solid and therefore will not substantially change in shape or form upon standing at room temperature for 24 hours. Similar processes that achieve the same result, such as drying, spray-drying, vacuum drying and vitrification, may also be employed, either alone or in combination.

As used herein, “moisture content” refers to levels determined by procedures substantially similar to the FDA-approved, modified Karl Fischer method (Centers for Biologics Evaluation and Research, FDA, Docket No. 89D-0140, 83-93; 1990 and references cited therein) or by near infrared spectroscopy. Suitable moisture content(s) for a particular inventive haemostatic material may be determined empirically by one skilled in the art depending upon the intended application(s) thereof.

For example, in certain embodiments of the present invention, higher moisture contents are associated with more flexible solid dressings. Thus, in solid dressings intended to be deformed in use, it may be preferred for the haemostatic material to have a moisture content of at least 6% and even more preferably In the range of 6% to 44%.

Similarly, in other embodiments of the present invention, lower moisture contents are associated with more rigid solid dressings. Thus, in solid dressings intended to be used as formed or cast, it may be preferred for the haemostatic material to have a moisture content of less than 6% and even more preferably in the range of 1% to 6%.

Accordingly, illustrative examples of suitable moisture contents for the inventive haemostatic materials include, but are not limited to, the following (each value being±0.9%); less than 53%; less than 44%; less than 28%; less than 24%; less than 16%; less than 12%; less than 6%; less than 5%; less than 4%; less than 3%, less than 2.5%; less than 2%; less than 1.4%; between 0 and 12%, non-inclusive; between 0 and 6%; between 0 and 4%; between 0 and 3%; between 0 and 2%; between 0 and 1%; between 1 and 16%; between 1 and 11%; between 1 and 8%; between 1 and 6%; between 1 and 4%; between 1 and 3%; between 1 and 2%; and between 2 and 4%.

The fibrinogen component in the haemostatic material may be any suitable fibrinogen known and available to those skilled in the art. The fibrinogen component may also be a functional derivative or metabolite of a fibrinogen, such the fibrinogen α, β and/or γ chains, soluble fibrin I or fibrin II, or a mixture of two or more thereof. A specific fibrinogen (or functional derivative or metabolite) for a particular application may be selected empirically by one skilled In the art. As used herein, the term “fibrinogen” is intended to include mixtures of fibrinogen and small mounts of Factor XIII/Factor XIIIa, or some other such transaminase. Such small amounts are generally recognized by those skilled in the art as usually being found in mammalian fibrinogen after it has been purified according to the methods and techniques presently known and available in the art, and typically range from 0.1 to 20 Units/mL.

Preferably, the fibrinogen employed as the fibrinogen component is a purified fibrinogen suitable for introduction Into a mammal. Typically, such fibrinogen is a part of a mixture of human plasma proteins which include Factor XIII/XIIIa and have been purified to an appropriate level and virally inactivated. A preferred aqueous solution of fibrinogen for preparation of a solid dressing contains around 37.5 mg/mL fibrinogen at a pH of around 7.4±0.1. Suitable fibrinogen for use as the fibrinogen component has been described in the art e.g. U.S. Pat. No. 3,716,645, and similar materials are commercially available, e.g. sources such as Sigma-Aldrich, Enzyme Research Laboratories, Haematologic Technologies and Aniara.

The fibrinogen component should be present in the inventive haemostatic materials in an amount effective to react with the fibrinogen activator and form sufficient fibrin to reduce the flow of fluid from wounded internal tissue. According to certain preferred embodiments of the present invention, when the haemostatic material is frozen, the fibrinogen component is present in an amount of from 4.70 mg to 18.75 mg (±0.009 mg) per square centimeter of the surface(s) of the haemostatic material intended to contact the wounded internal tissue. *******

According to other preferred embodiments, when the haemostatic material is a solid, regardless of form, the fibrinogen component is present in an amount of from 5.00 mg to 450.00 mg (±0.009 mg) per square centimeter of the surface(s) intended to contact the wounded internal tissue being treated. Greater or lesser amounts, however, may be employed depending upon the particular application intended for the solid dressing.

For example, when the haemostatic material is in the shape of a rod or cylinder, the fibrinogen component is more preferably present in an amount of from 25.00 mg to 75.00 mg (±0.009 mg) per square centimeter of the surface(s) intended to contact the wounded internal tissue being treated. Alternatively, when the haemostatic material is in the shape of a flat sheet or disk, the fibrinogen component is more preferably present in an amount of from 5.00 to 56.00 mg (±0.009 mg) per square centimeter of the surface(s) intended to contact the wounded internal tissue being treated. Still alternatively, when the haemostatic material is powdered, either loose or compressed, the fibrinogen component is more preferably present in an amount from 26.00 mg to 450.00 mg (±0.09 mg) per square centimeter of the surface(s) intended to contact the wounded internal tissue being treated.

The fibrinogen activator employed in the haemostatic materials of the present invention may be any of the substances or mixtures of substances known by those skilled in the art to convert fibrinogen (or a fibrinogen equivalent) into fibrin, illustrative examples of suitable fibrinogen activators include, but are not limited to, the following: thrombins, such as human thrombin or bovine thrombin, and prothrombins, such as human prothrombin or prothrombin complex concentrate (a mixture of Factors II, VII, IX and X); snake venoms, such as batroxobin, reptilase (a mixture of batroxobin and Factor XIIIa), bothrombin, calobin, fibrozyme, and enzymes isolated from the venom of Bothrops jararacussu; and mixtures of any two or more of these. See, e.g., Dascombe et al., Thromb. Haemost. 78:947-51 (1997); Hahn et al., J. Biochem. (Tokyo) 119:835-43 (1996); Fortova et al., J. Chromatogr. S. Biomed. Appl. 694:49-53 (1997); and Andriao-Escarso et al., Toxicon. 35: 1043-52 (1997).

Preferably, the fibrinogen activator is a thrombin. More preferably, the fibrinogen activator is a mammalian thrombin, although bird and/or fish thrombin may also be employed in appropriate circumstances. While any suitable mammalian thrombin may be used, the thrombin employed is preferably a lyophilized mixture of human plasma proteins which has been sufficiently purified and virally inactivated for the intended use of the solid dressing. Suitable thrombin is available commercially from sources such as Sigma-Aldrich, Enzyme Research Laboratories, Haematologic Technologies and Biomol International. A particularly preferred aqueous solution of thrombin for preparing the inventive haemostatic materials contains thrombin at a potency of between 10 and 2000±50 International Units/mL, and more preferred at a potency of 25±2.5 International Units/mL. Other constituents may include albumin (generally about 0.1 mg/mL) and glycine (generally about 100 nM±0.1 nM). The pH of this particularly preferred aqueous solution of thrombin is generally in the range of 6.5-7.8 and preferably 7.4±0.1, although a pH in the range of 5.5-8.5 may be acceptable.

In addition, to the inventive haemostatic material(s), the solid and frozen dressings of the present invention may optionally further comprise one or more support materials. As used herein, a “support material” refers to a material that sustains or improves the structural integrity of the solid or frozen dressing and/or the fibrin clot formed when such a dressing is applied to wounded tissue. The support material may be an internal support material or a surface support material. Moreover, in the case of the latter, if the dressing is in a form that has a wound facing side, the support material may fee on the wound facing side or it may be on the non-wound facing side or both.

Any suitable resorbable material known and available to those skilled in the art may be employed in the present invention. For example, the resorbable material may be a proteinaceous substance, such as silk, fibrin, keratin, collagen and/or gelatin. Alternatively, the resorbable material may be a carbohydrate substance, such as alginates, chitin, cellulose, proteoglycans (e.g. poly-N-acetyl glucosamine), glycolic acid polymers, lactic acid polymers, or glycolic acid/lactic acid co-polymers. The resorbable material may also comprise a mixture of proteinaceous substances or a mixture of carbohydrate substances or a mixture of both proteinaceous substances and carbohydrate substances. Specific resorbable material(s) may be selected empirically by those skilled in the art depending upon the intended use of the solid dressing.

According to certain preferred embodiments of the present invention, the resorbable material is a carbohydrate substance. Illustrative examples of particularly preferred resorbable materials include, but are not limited to, the materials sold under the trade names Vicryl™ (a glycolic acid/lactic acid copolymer) and Dexon™ (a glycolic acid polymer).

Any suitable non-resorbable material known and available to those skilled in the art may be employed as the support material. Illustrative examples of suitable non-resorbable materials include, but are not limited to, plastics, silicone polymers, paper and paper products, latex, gauze plastics, non-resorbable suture materials, latexes and suitable derivatives thereof.

According to other preferred embodiments, the support material comprises an internal support material. Such an internal support material is preferably fully contained within the haemostatic material(s) of a solid or frozen dressing. The internal support material may take any form suitable for the intended application of the haemostatic material. For example, according to certain embodiments, the internal support material may be particles of a predetermined suitable size which are dispersed throughout the haemostatic material. Alternatively, a sheet or film or internal support material may be included in the solid or frozen haemostatic material.

According to still other preferred embodiments, the support material may comprise a backing material on the surface(s) of the dressing opposite the wound-facing surface. As with the internal support material the backing material may be a resorbable material or a non-resorbable material, or a mixture thereof, such as a mixture of two or more resorbable materials or a mixture of two or more non-resorbable materials or a mixture of resorbable material(s) and non-resorbable material(s).

According to still other preferred embodiments, the dressing comprises both a backing material and an internal support material in addition to the haemostatic material(s). According to still other preferred embodiments, the dressing comprises both a front support material and an internal support material in addition to the haemostatic layer(s). According to still other preferred embodiments, the dressing comprises a backing material, a front support material and an internal support material in addition to the haemostatic layer(s).

According to certain preferred embodiments, the haemostatic material(s) may also contain a binding agent to maintain the physical integrity of the haemostatic material(s). Illustrative examples of suitable binding agents include, but are not limited to, sucrose, mannitol, sorbitol, gelatin, hyaluron and its derivatives, such as hyaluronic acid, maltose, povidone, starch, chitosan and its derivatives, and cellulose derivatives, such as carboxymethylcellulose, as well as mixtures of two or more thereof.

According to certain embodiments of the present invention, particularly where the solid or frozen dressing is manufactured using a mold, the dressings may also optionally further comprise a release layer in addition to the haemostatic material(s) and support layer(s). As used herein, a “release layer” refers to a layer containing one or more agents (“release agents”) which promote or facilitate removal of the solid or frozen dressing from a mold in which it has been manufactured. A preferred such agent is sucrose, but other suitable release agents include gelatin, hyaluron and its derivatives, including hyaluronic acid, mannitol, sorbitol and glucose. Alternatively, such one or more release agents may be contained in the haemostatic material.

The haemostatic material and any layer(s) may be affixed to one another by any suitable means known and available to those skilled in the art. For example, a physiologically-acceptable adhesive may be applied to a backing material (when present), and the haemostatic material subsequently affixed thereto.

In certain embodiments of the present invention, the physiologically-acceptable adhesive has a shear strength and/or structure such that the backing material can be separated from the fibrin clot formed by the haemostatic layer after application of the dressing to wounded tissue. In other embodiments, the physiologically-acceptable adhesive has a shear strength and/or structure such that the hacking material cannot be separated from the fibrin clot after application of the bandage to wounded tissue.

Suitable fibrinogen components and suitable fibrinogen activators for the haemostatic materials may fee obtained from any appropriate source known and available to those skilled in the art, including, but not limited to, the following: from commercial vendors, such as Sigma-Aldrich and Enzyme Research Laboratories; by extraction and purification from human or mammalian plasma by any of the methods known and available to those skilled in the art; from supernatants or pastes derived from plasma or recombinant, tissue culture, viruses, yeast, bacteria, or the like that contain a gene that expresses a human or mammalian plasma protein which has been introduced according to standard recombinant DMA techniques; and/or from the fluids (e.g. blood, milk, lymph, urine or the like) of transgenic mammals (e.g. goats, sheep, cows) that contain a gene which has been introduced according to standard transgenic techniques and that expresses the desired fibrinogen and/or desired fibrinogen activator.

According to certain preferred embodiments of the present invention, the fibrinogen component is a mammalian fibrinogen such as bovine fibrinogen, porcine fibrinogen, ovine fibrinogen, equine fibrinogen, caprine fibrinogen, feline fibrinogen, canine fibrinogen, murine fibrinogen or human fibrinogen. According to other embodiments, the fibrinogen component is bird fibrinogen or fish fibrinogen. According to any of these embodiments, the fibrinogen component may be recombinantly produced fibrinogen or transgenic fibrinogen.

According to certain preferred embodiments of the present invention, the fibrinogen activator is a mammalian thrombin, such as bovine thrombin, porcine thrombin, ovine thrombin, equine thrombin, caprine thrombin, feline thrombin, canine thrombin, murine thrombin and human thrombin. According to other embodiments, the thrombin is bird thrombin or fish thrombin. According to any of these embodiments, the thrombin may be recombinantly produced thrombin or transgenic thrombin.

As a general proposition, the purity of the fibrinogen component and/or the fibrinogen activator for use in the solid dressing will be a purity known to one of ordinary skill in the relevant art to lead to the optimal efficacy and stability of the protein(s). Preferably, the fibrinogen component and/or the fibrinogen activator has been subjected to multiple purification steps, such as precipitation, concentration, diafiltration and affinity chromatography (preferably immunoaffinity chromatography), to remove substances which cause fragmentation, activation and/or degradation of the fibrinogen component and/or the fibrinogen activator daring manufacture, storage and/or use of the solid dressing. Illustrative examples of such substances that are preferably removed by purification include: protein contaminants, such as inter-alpha trypsin inhibitor and pre-alpha trypsin inhibitor; non-protein contaminants, such as lipids; and mixtures of protein and non-protein contaminants, such as lipoproteins. The fibrinogen component and/or fibrinogen activator and/or the inventive haemostatic materials may also be subjected to suitable, sterilization treatments, including, but not limited to, treatment with one or more of the following, heat, gamma radiation, e-beam radiation, plasma radiation and ethylene oxide.

The amount of the fibrinogen activator employed in the solid dressing is preferably selected to optimize both the efficacy and stability thereof. As such, a suitable concentration for a particular application of the solid dressing may be determined empirically by one skilled in the relevant art.

According to certain preferred embodiments of the present invention, when the fibrinogen activator is human thrombin, the amount of human thrombin employed is between 0.03 and 16.10 Units (all values being±0.009) per square centimeter of the surface(s) of the haemostatic material intended to contact the wounded internal tissue. Greater or lesser amounts, however, may be employed depending upon the particular application intended for the solid dressing.

For example, when the haemostatic material is a solid in the shape of a rod or cylinder, the fibrinogen activator is more preferably present in an amount of from 2.50 Units to 7.50 Units (±0.009 Units) per square centimeter of the surface(s) intended to contact the wounded internal tissue being treated. Alternatively, when the haemostatic material is a solid in the shape of a flat sheet or disk, the fibrinogen activator is more preferably present in an amount of from 0.03 Units to 16.10 Units (±0.009 Units) per square centimeter of the surface(s) intended to contact the wounded internal tissue being treated. Still alternatively, when the haemostatic material is a powdered solid, either loose or compressed, the fibrinogen activator is more preferably present in an amount of about 1.3 Units (±0.09 mg) per square centimeter of the surface(s) intended to contact the wounded internal tissue being treated. Still alternatively, when the haemostatic material is frozen, the fibrinogen activator is more preferably present in an amount of about 1.3 Units (±0.09 mg) per square centimeter of the surface(s) intended to contact the wounded internal tissue being treated.

According to still other preferred embodiments of the present invention, when the fibrinogen activator is human thrombin, the amount of human thrombin employed is between 0.0087 and 1.0000 Units (all values being±0.00009) per milligram of the fibrinogen component. Greater or lesser amounts, however, may be employed depending upon the particular application intended for the solid dressing.

For example, when the haemostatic material is a solid in the shape of a rod or cylinder, the fibrinogen activator is more preferably present in an amount of about 0.1 Units (±0.09 Units) per milligram of the fibrinogen component. Alternatively, when the haemostatic material is a solid in the shape of a flat sheet or dish, the fibrinogen activator is more preferably present in an amount of from 0.1 Units to 1.00 Units (±0.009 Units) per milligram of the fibrinogen component. Still alternatively, when the haemostatic material is a powdered solid, either loose or compressed, the fibrinogen activator is more preferably present in an amount of about 0.0087 Units to 0.0500 Units (±0.00009 Units) per milligram of the fibrinogen component. Still alternatively, when the haemostatic material is frozen, the fibrinogen activator is more preferably present in an amount of about 0.07 Units to 0.10 Units (±0.009 Units) per milligram of the fibrinogen component.

During use of the inventive haemostatic materials, the fibrinogen component and the fibrinogen activator are preferably activated at the time the dressing is applied to the wounded tissue by the endogenous fields of the patient escaping from the hemorrhaging wound. Alternatively, in situations where fluid loss from the wounded tissue is insufficient to provide adequate hydration of the protein layers, the fibrinogen component and/or the fibrinogen activator may be activated by a suitable, physiologically-acceptable liquid, optionally containing any necessary co-factors and/or enzymes, prior to or during application of the dressing to the wounded tissue.

In some embodiments of the present invention, the inventive haemostatic materials may also contain one or more supplements, such as growth, factors, drugs, polyclonal and monoclonal antibodies and other compounds. Illustrative examples of such supplements include, but are not limited to, the following; fibrinolysis inhibitors, such as aprotonin, tranexamic acid and epsilon-amino-caproic acid; antibiotics, such as tetracycline and ciprofloxacin, amoxicillin, and metronidazole; anticoagulants, such as activated protein C, heparin, prostacyclins, prostaglandins (particularly (PGI₂), leukotrienes, antithrombin III, ADPase, and plasminogen activator; steroids; such as dexamethasone, inhibitors of prostacyclin, prostaglandins, leukotrienes and/or kinins to inhibit inflammation; cardiovascular drugs, such as calcium channel blockers, vasodilators and vasoconstrictors, such as epinephrine; chemoattractants; local anesthetics such as bupivacaine; and antiproliferative/antitumor drugs such as 3-fluorouracil (5-FU), taxol and/or taxotere; antivirals, such as gangcyclovir, zidovudine, amantidine, vidarabine, ribaravin, trifluridine, acyclovir, dideoxyuridine and antibodies to viral components or gene products; cytokines, such as alpha- or beta- or gamma-Interferon, alpha- or beta-tumor necrosis factor, and interleukins; colony stimulating factors; erythropoietin; antifungals, such as diflucan, ketaconizole and nystatin; antiparasitic gents, such as pentamidine; and anti-inflammatory agents, such as alpha-1-anti-trypsin and alpha-1-antichymotrypsin; anesthetics, such as bupivacaine; analgesics; antiseptics; hormones; vitamins and other nutritional supplements; glycoproteins; fibronectin; peptides and proteins; carbohydrates (both simple and/or complex); proteoglycans; antiangiogenins; antigens; lipids or liposomes; oligonucleotides (sense and/or antisense DNA and/or RNA); and gene therapy reagents. In other embodiments of the present invention, the backing layer and/or the internal support layer, if present, may contain one or more supplements. According to certain preferred embodiments of the present invention, the therapeutic supplement is present in an amount greater than its solubility limit in fibrin.

The inventive haemostatic materials, and the solid and frozen dressings containing them, may be applied to any internal wounded tissue in a mammal using any of the suitable techniques and/or devices known and available to one skilled in the medical arts. For example, when used to treat vascular punctures, the haemostatic material(s) may be applied via a catheter, either with or without a guide wire. The inventive materials and dressings may also be applied in conjunction with endoscopic techniques, including endoscopic surgery, laparoscopic surgery and tele-robotic/tele-presence surgery. According to such embodiments, it is preferable to use a “plunger” or “tamper” to facilitate passage of the inventive materials through surrounding tissue to the wounded internal tissue being treated. The inventive materials and dressings may also be applied manually.

As used herein the terms “kittner” and or “Kittner”, singular or pleural, refers to a device resembling the conventional endoscopic surgical tissue probe or dividing device that has a shaft and an end intended to manipulate or apply pressure to the patient's tissues. As used herein, such the term may also apply to a similarly-shaped device that is tipped with some form of hemostatic mixture or product to be applied to injured tissue.

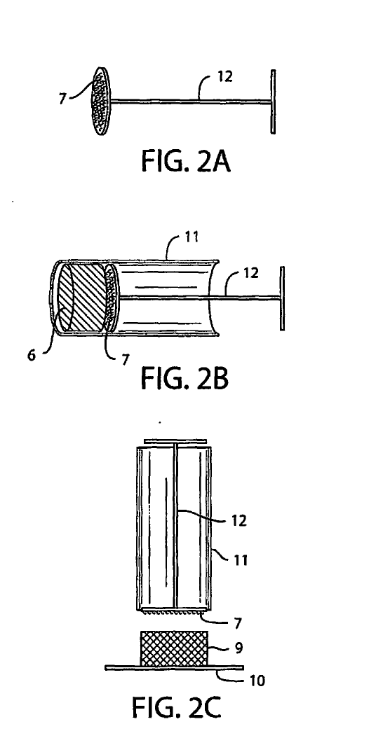

For example, in view of FIG. 2. FIG. 2A depicts a plunger 12 having disposed on one end a hook or loop material 7. In view of FIG. 2B, the plunger 12 has a shaft and an end having the hook or loop material 7 disposed of on said end. The end having the hook or loop material 7 is of such a diameter to fit tightly into the tube 11. Attached to said hook or loop material 7 is a lyophilized haemostatic bandage as made by the methods described herein. A simple example of such tube and plunger is a standard 3 ml or 10 ml syringe wherein the opening of the syringe is not narrowed, and wherein the plunger fits tightly into the tube of the syringe. Added to the bottom of the plunger and facing the opening of the syringe would be the hook or loop material 7. The haemostatic material 6 can then be manufactured in the tube, as described herein.

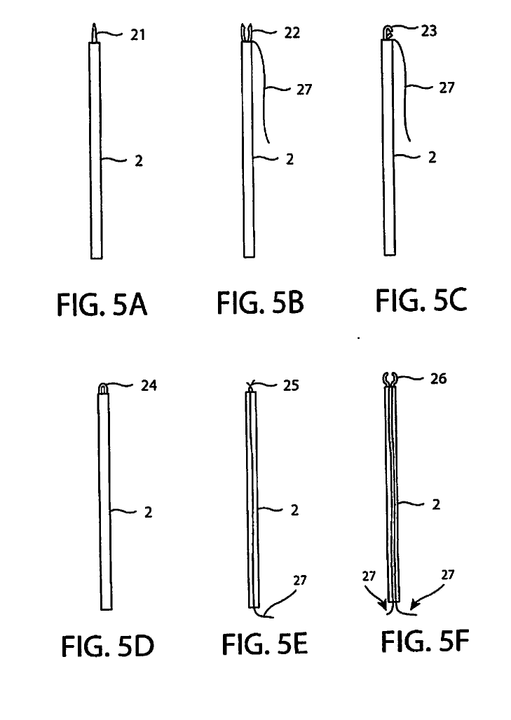

Alternatively, a manufactured haemostatic material 6 can be manufactured in a different vessel and secured to the applicator via an attachment means, including, but not limited to a hook or loop material 7 and placed into the mechanism depicted herein. Other suitable attachment means are depicted in FIGS. 5A-5F.

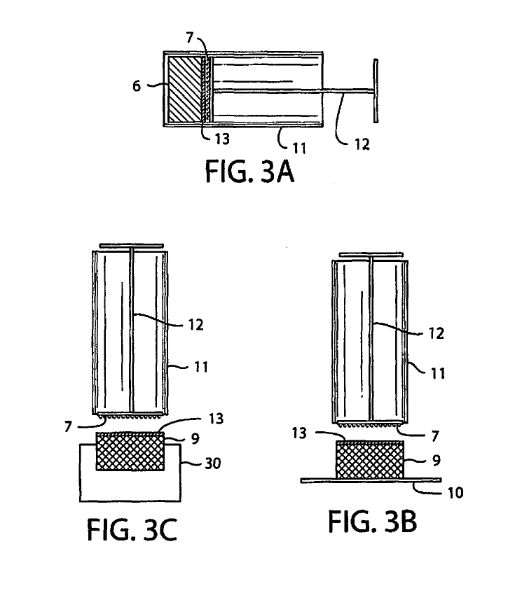

FIG. 3 depicts a similar embodiment, however further utilizing a resorbable backing on the haemostatic material 6. Accordingly, the haemostatic material 6 may be manufactured within the tube 11, or manufactured in a different vessel and then secured to the hook or loop material 7. In view of FIG. 3B, after contact with a wound surface and contact with an aqueous fluid, the haemostatic material forms fibrin and the hook material 7, tube 11, and plunger 12 can be pulled free, The plunger is further capable of applying a force to the wound surface to aid in securing the haemostatic material to the wound surface.

In view of FIG. 3C, the haemostatic material 9, being contacted with a wound surface 30, reacts and forms fibrin and the haemostatic material is secured to the wound surface 30 allowing the hook or loop material 7, plunger 12, and tube 11 to be pulled free. Before pulling free, the plunger may be utilized to impart force upon the wound surface. The plunger and tube 11 may be short or long, depending on the necessary application within the body, for example, it may be used in an endoscope. Where the wound is a deep wound and of three dimensions, the haemostatic material can be inserted into said wound to fill the wound opening. Accordingly, in view of FIG. 3C, the wound is a cavity 30 that is filled with a haemostatic material 6 (as in 3A) and forms fibrin 9 upon contact with the wound surface.

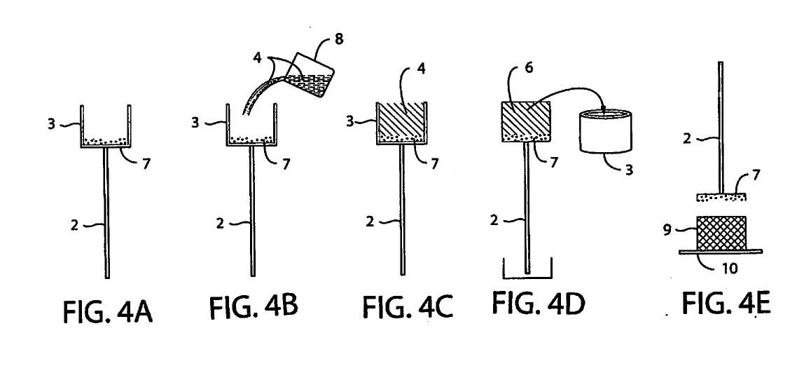

In view of FIG. 4, a depiction of a method of adhering a haemostatic bandage to an applicator 2, wherein a sheath, 3, is situated on the applicator 2, and a material 7 is adhered to the bottom of the applicator. FIG. 4B identifies that liquid fibrinogen and thrombin mixture is poured into the sheath, see FIG. 4C. The liquid 4 is then frozen and lyophilized and the sheath 3 can be removed as in FIG. 4D. Finally, application of the haemostatic bandage to a wound surface 10 provides that the haemostatic material reacts with an aqueous fluid and forms fibrin 9.

The hook or loop material 7 in FIGS. 4A-4E may include other suitable materials and may be an absorbant material, but it is also conceived to be utilized with ordinary fast dressings as described in the description and embodiments herein.

FIGS. 5A-F depict various connectivity mechanisms that may be attached to the end of a plunger or an applicator 2. For example, a pin or pins 21 may simply stick into the haemostatic material. FIG. 5B depicts tweezers 22 or other similar multi prong clamping mechanism may hold onto the haemostatic material or the backing. An action mechanism 27 may move the tweezers or clamping type mechanism. FIG. 5C depicts latch 23 may hook on the haemostatic material and be released via a release mechanism 27. FIG. 5D depicts a staple 24 made of metal, plastic, or other resorbable or non-resorbable material, wherein the staple 24 secures the haemostatic material, or backing to the plunger or applicator. FIG. 5E depicts a suture 25, and wherein said release mechanism is depicted in a hollow applicator 2. Like a staple seeks to secure the haemostatic material or the backing to the plunger or applicator. A release mechanism 27 may pull out the suture to aid in release. Finally, FIG. 5F depicts a clamp mechanism 26, like the tweezers 22, assumes the ability to hold onto the haemostatic material and then release said material via a release mechanism 27 disposed of through the hollow applicator 2. These types of tweezers and release mechanisms are well known in the art of laparoscopic surgery and may be suitable for use in the embodiments described herein.

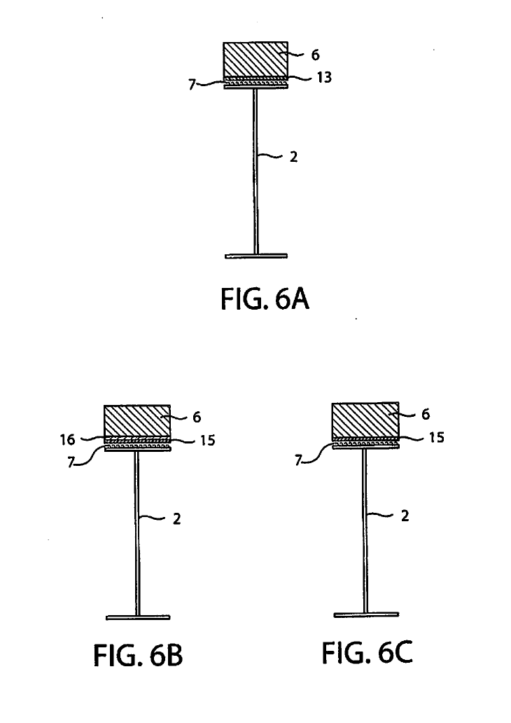

In view of FIG. 6, three further depictions show a haemostatic material 6 as secured to an applicator 2, and having in FIG. 6A a resorbable backing 13, in FIG. 6B a release layer 16 and a non-resorbable material 15 and in FIG. 6C a non-resorbable backing 15. The haemostatic material 6 is secured to an applicator. It is conceived that various shaped haemostatic materials or flat haemostatic materials may appropriately be utilized with the various applicators and syringe type applicators as described and depicted in these figures, in the Examples, and in the description herein. These haemostatic material may be manufactured as attached to the applicator or manufactured separately and attached to the applicator prior to use.

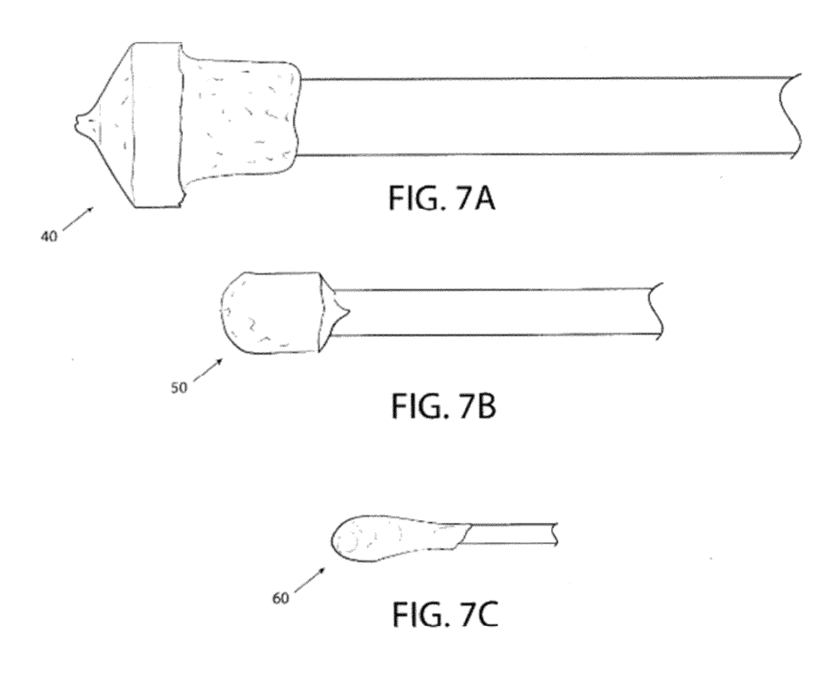

In view of FIGS. 7A, 7B, and 7C, three different applicators and absorbent tips are depicted. It is understood that while these are three examples, other shapes and sizes are appropriate in surgical and trauma situations.

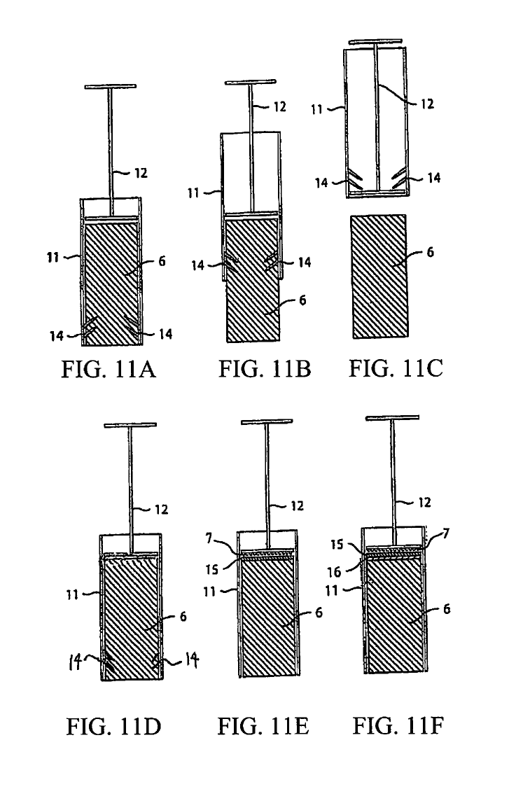

In view of FIG. 11, a depiction of a rod haemostatic material 6 as situated within a tube, wherein the inside of the tube comprises quills 14 that secured the lightweight haemostatic material 6 in place. Upon pressure from the plunger 12, the quills 14, are forced into the other direction and allowing the haemostatic material 6 to be pressed out of the tube 11. Accordingly, the quills are of a material suitable to hold the light weight of the haemostatic material 6, but are sufficiently flexible or hinged to be forced into a different direction, thus allowing the haemostatic material 6 to slide out of the tube, as is depicted in FIGS. 11B and finally 11C.

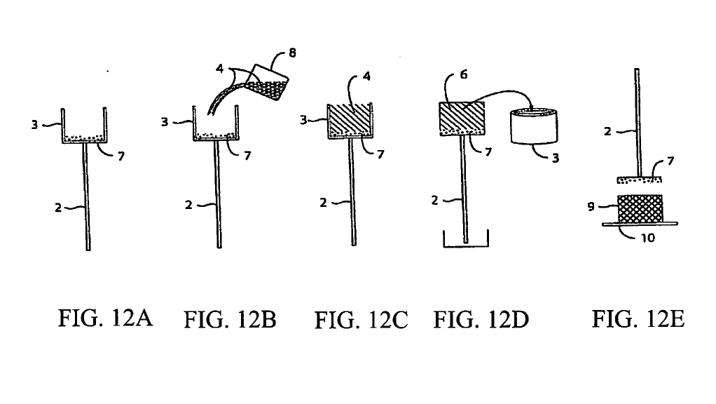

FIG. 11D depicts a haemostatic dressing 6 in a tube 11 wherein said haemostatic dressing having quills depicted as facing the same direction as the travel of the haemostatic material when it is expelled. The quills may still deflect, but do not invert, as in the description of FIGS. 11A, 11B, and 11C. Each of the quills shown in FIGS. 11A-11D are shown enlarged for purposes of depicting the mechanism. The size of the quills 14 is necessary only to fix the haemostatic material in place within the tube 11. The quills 14 may be plastic, metal or other suitable resorbable or non-resorbable material and are intended to fix the material in place, but to allow the haemostatic material to be expelled without significant damage. FIG. 11E depicts a non-resorbable backing 15 connected to said plunger via any of the connective means depicted in FIGS. 2 or 5. Alternatively, a resorbable backing 13 may be utilized in place of the non-resorbable backing 15, Similarly FIG. 11F is a non-resorbable backing 15 that comprises a release layer 16 between said non-resorbable backing and said haemostatic material 6. In view of FIGS. 12A-12E, an embodiment is depicted as one possible mechanism to manufacture a rod shaped haemostatic bandage wherein a sheath 3 is placed at the end of an applicator 2, and a hook or loop material 7 (one option for any number of backing materials) is secured to the bottom of the applicator. A liquid mixture of fibrinogen component and fibrinogen activator 4 is poured from a vessel 8 in to the sheath 3. The liquid mixture 4 is then frozen in place, and ultimately lyophilized. Accordingly, in view of FIG. 12D, the sheath 3 can be removed from the dry, solid cylindrical haemostatic material that is attached to the end of the applicator 2. FIG. 12E further depicts that the applicator 2 can be pressed onto a wound surface 10 and the haemostatic material forms fibrin 9, allowing the applicator 2 to be removed.

The following examples are illustrative only and are not intended to limit the scope of the Invention as defined by the appended claims. It will be apparent to those skilled in the art that various modifications and variations can be made in the methods of the present invention without departing from the spirit and scope of the invention. Thus, it is intended that the present invention cover the modifications and variations of this invention provided they come within the scope of the appended claims and their equivalents.

### EXAMPLES

The following is a list of acronyms used in the Examples below:
CFB: Complete Fibrinogen Buffer (100 mM Sodium Chloride, 1.1 mM Calcium Chloride, 10 mM Tris, 10 mM Sodium Citrate, 1.5% Sucrose, Human Serum Albumin (80 mg/g of total protein) and Tween™ 80 (non-animal source) 15 mg/g total protein)
CTB: Complete Thrombin Buffer (150 mM Sodium Chloride, 40 mM Calcium Chloride, 10 mM Iris and 100 nM L-Lysine with the addition of HSA at 100 ug/ml)
ERL: Enzyme Research Laboratories
EVPA: Ex Vivo Porcine Arteriotomy
EVPCA: Ex Vivo Porcine Carotid Arteriotomy
FD: Inventive haemostatic dressing
HSA: Human Serum Albumin
HD: A “sandwich” fibrin sealant haemostatic dressing as disclosed in U.S. Pat. No. 6,762,336
IFB: Incomplete Fibrinogen Buffer; CFB without HSA and Tween
Fibrinogen Dose: In a solid mass, the amount of fibrinogen within the mass divided by the surface area to be treated. Usually expressed in mg of Fibrinogen per cm², where the mass of fibrinogen is determined via a clottable protein assay
PETG: Glycol-modified Polyethylylenetetrapthalate
PPG: Polypropylene
PVC: Poly vinyl chloride
T:F Thrombin to Fibrinogen ratio. In a test article, the amount of thrombin activity per unit of fibrinogen. Usually expressed in thrombin NIH Units per mg of fibrinogen (measured via a clottable protein assay)
Thrombin Dose: In a solid mass, the amount of thrombin within the mass divided by the surface area to be treated. Usually expressed in NIH Units of thrombin per cm²
TRIS: trishydroxymethylaminomethane (2-amino-2-hydroxymethyl-1,3-propanediol)

The ability of the dressings to seal an injured blood vessel was determined by modifications of an ex two porcine arteriotomy (EVPA) performance test, which was first described in U.S. Pat. No. 6,762,336. The EVPA performance test evaluates the ability of a dressing to stop fluid flow through a hole in a porcine artery. While the procedure described in U.S. Pat. No. 6,762,336 has been shown to be useful for evaluating haemostatic dressings, it failed to replicate faithfully the requirements for success in vivo. More specifically, the procedure disclosed in U.S. Pat. No. 6,762,336 required testing at 37° C., whereas, in the real world, wounds are typically cooler than that. This decreased temperature can significantly reduce the rate of fibrin formation and its haemostatic efficacy in trauma victims. See, e.g., Acheson et al., J. Trauma 59:865-874 (2005). The test in U.S. Pat. No. 6,762,336 also failed to require a high degree of adherence of the dressing to the injured tissue. A failure mode in which fibrin forms but the dressing fails to attach tightly to the tissue would, therefore, not be detected by this test. Additionally, the pressure utilized in the procedure (200 mHg) may be exceeded during therapy for some trauma patients. The overall result of this is that numerous animal tests, typically involving small animals (such as rats and rabbits), must be conducted to accurately predict dressing performance in large animal, realistic trauma studies and in the clinical environment.

In order to minimize the amount of time and the number of animal studies required to develop the present invention, an improved ex vivo testing procedure was developed. To accomplish this, the basic conditions under which the dressing test was conducted were changed, and the severity of the test parameters was increased to include testing at lower temperatures (i.e. 29-33° C. vs. 37° C., representing the real physiologic challenge at realistic wound temperatures (Acheson et al., J. Trauma 59:865-874 (2005)), higher pressures (i.e. 250 mmHg vs. 200 mmHg) a longer test period (3 minutes vs. 2 minutes) and larger sized arterial injuries (U.S. Pat. No. 6,762,336 used an 18 gauge needle puncture, whereas the revised procedure used puncture holes ranging from 2.8 mm to 4 mm×6 mm).

In addition, a new test was derived to directly measure adherence of the dressing to the injured tissue.

Figures and drawings are included within the body of this application and are intended to be part of the application as filed.

### Example 1

In order to apply the haemostatic test articles to the surface of as injured artery surrounded fey a tissue stimulant, the test articles were housed in cylindrical molds made of 10 or 3 mL polypropylene syringes (Becton Dickinson) with the luer-lock end removed. The plungers were withdrawn to the 6 mL and 2 mL mark respectively. For dressings utilizing a backing, the support material was cut and placed into each mold and pushed down until it was adjacent to the plunger. Once prepared the molds were placed upright and surrounded by dry ice, leaving the opening exposed at the top. 1 ml of fibrinogen and 0.15 mL of thrombin (with or without backing material dispersed within) were dispensed into the 10 mL molds and 1 ml of fibrinogen and 0.15 mL of thrombin (with or without support material dispersed within) were dispensed into the 3 mL molds, which were allowed to freeze for 5 minutes. The molds were then placed into the −80° C. freezer for at least two hours before being placed into a pre-cooled Genesis™ lyophylizer (Virtis, Gardiner, N.Y.). The chamber was sealed and the temperature equilibrated. The chamber was then evacuated and the dressings lyophilized via a primary and secondary drying cycle.

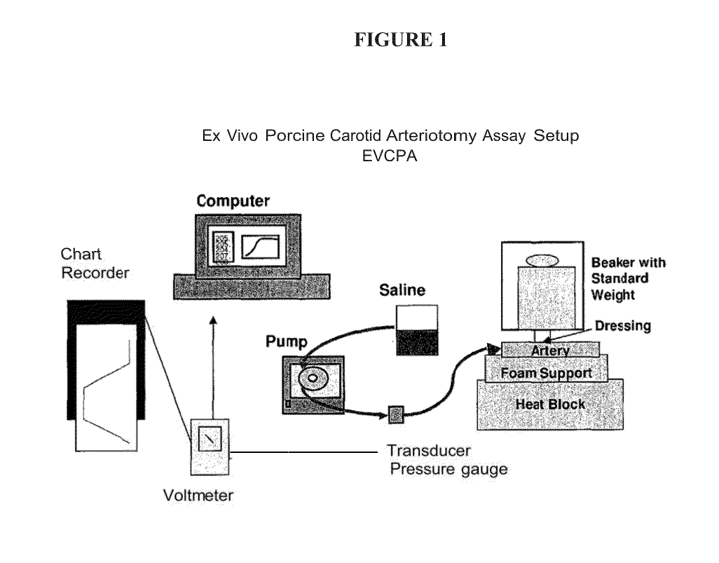

They were subsequently performance tested in a modified EVPA assay (Deep Tissue EVPA). Briefly, in one version, a plastic foam form was slipped over the artery. This covering had a hole in it that corresponded to the hole in the artery and the surrounding tissue (FIG. 1). In another variant, the foam was replaced with a piece of tissue, specifically, bovine muscle, in which a hole had been prepared as with the foam. The foam was maintained at 37° C. by placement in a 37° C. water bath, while the muscle tissue was maintained at 37° C. by placement on a 37° C. block heater. Warm saline was added to the surface of the dressing and the mold was immediately passed down thru the hole in the foam to the artery surface. The plunger was then depressed and held by hand for 3 minutes, after which the mold was withdrawn as the plunger was depressed further. At this point the artery was pressurized and the assay continued as described hereafter.

### Deep Tissue EVPA Testing

Equipment and Supplies:
In-line high pressure transducer (Ashcroft Duralife™ or equivalent)
Peristaltic pump (Pharmacia Biotech™, Model P-1 or equivalent)
Voltmeter (Craftsman™ Professional Model 82324 or equivalent)
Computer equipped with software for recording pressure or voltage information
Tygon™ tubing (assorted sizes) with attachments
Water bath (Baxter Durabath™ or equivalent), preset to 37° C.
Incubation chamber (VWR™, Model 1400G or equivalent), preset to 37° C.
Thermometer to monitor temperatures of both water bath and oven
Assorted forceps, hemostats, and scissors
10 cc, and 20 cc syringes with an approximately 0.6 cm hole drilled in center and smaller hole drilled through both syringe and plunger. This hole, drilled into the end of the syringe, will be used to keep the plunger drawn hack and stationary.
O-rings (size 10 and 13)
Plastic Shields to fit the 10 cc and 20 cc syringes (approximately 3.5 cm in length)
P-1000 Pipetman™ with tips
Programmable Logic Controller (PLC) to control the pumps to maintain the desired pressure profile (Optional, Manual control may be used if desired.)

### 1. Materials and Chemicals

Porcine descending aortas (Pel-Freez Biologicals™, Catalog #59402-2 or equivalent)
Cyanoacrylate glue (Vetbond™, 3M or equivalent)
18-gauge needle(s)
0.9% Saline, maintained at 37° C.
Red food coloring
Vascular Punch(es), 2.8 mm or other

Plastic Wrap

### 2. Artery Cleaning and Storage

Store arteries at −20° C. until used.

Thaw arteries at 37° C. in H2O bath.

Clean fat and connective tissue from exterior surface of artery.

Cut the arteries into ˜5 cm segments.

The arteries may be refrozen to −20° C. and stored until use.

### 3. Artery Preparation for Assay

Turn the artery inside-out so that the smooth, interior wall is lacing outwards.

Stretch a size 13 O-ring over a 20 cc syringe or a size 10 O-ring over a 10 cc syringe with an approximately 0.6 cm (0.25 in) hole drilled into one side.

Pull the artery onto the syringe, taking care not to tear the artery, or have a too loose fit. The artery should fit snugly to the syringe. Slide another O-ring of the same size onto the bottom of the syringe

Carefully poll both O-rings over the ends of the artery. The distance between the O-rings should he at least 3.5 cm

Using the blade of some surgical scissors, gently scrape the surface of the artery in order to roughen fee surface of the artery.

Use a 18-gauge needle to poke a hole through the artery over the site of the hole in the syringe barrel (see note above)

The tip of the biopsy punch is inserted through the hole in the artery. Depress the punch's plunger to make an open hole in the artery. Repeat a couple of times to ensure that the hole is open and free of connective tissue.

Patch holes left by collateral arteries. Generally this is done by cutting a patch from a latex glove and gluing it over the hole with cyanoacrylate glue. Allow the glue to cure for at least 10 minutes.
Place the artery in the warmed, moistened container and place in the incubation chamber. Allow the arteries to warm for at least 30 minutes.

### 4. Solution and Equipment Preparation

1. Check to see that the water bath, block heater and incubation chamber are maintained at 37° C.
2. Make sure that there is sufficient 0.9% saline in the pump's reservoir for completion of the day's assays. Add more if needed.
3. Place 0.9% saline and 0.9% saline with a few drops of red food coloring added into containers in a water bath so that the solutions will he warmed prior to performing the assay.
4. Prepare the container for warming the arteries in the incubation chamber by lining with KimWipes™ and adding a small amount of water to keep the arteries moist.
5. Cheek the tubing for air bubbles. If bubbles exist, turn on fee pump sod allow the 0.9% saline to flow until all bubbles are removed.

### 5. Application of the Dressing

Slip either the warmed (at 37° C.) plastic foam form or the warmed tissue over the artery. Align the hole in it to correspond to the hole in the artery and the surrounding tissue (FIG. 1).

Open fee haemostatic dressing (Test Article) pouch and remove haemostatic dressing & Applicator.

Slowly wet the haemostatic dressing drop wise with 0.9% saline warmed to 29-33° C. or other blood substitute, taking care to keep the saline from running off the edges. Any obvious differences in wetting characteristics from the positive control should be noted on the data collection forms.
NOTE: By way of example, a representative (13-15 mg/cm2 of fibrinogen) 2.4×2.4 cm haemostatic dressing should generally be wet with 800 μl of saline or other blood substitute. The amount of saline used can be adjusted depending on the requirements of the particular experiment being performed; however, any changes-should be noted on the data collection forms.

Immediately pass the dressing in the applicator down thru the hole in the foam to the artery surface. Depress the plunger by hand and hold by hand for 3 minutes, after which the applicator Is withdrawn as the plunger was depressed further.

After polymerization, note the condition of the haemostatic dressing. Any variation from the positive control should be noted on the data collection form.

EXCLUSION CRITERION: The mesh support material must remain over the hole in the artery. If it has shifted during the polymerization and does not completely cover the hole the haemostatic dressing must be excluded.

### Testing Procedure

### 1. Diagram of Testing Equipment Set-Up

The set-up of the testing equipment is shown in FIG. 1. Some additional, unshown components may be utilized to read out (pressure gauge) or control the pressure within the system

### 2. Equipment and Artery Assembly

Fill the artery and syringe with red 0.9% saline warmed to 37° C. taking care to minimize the amount of air bubbles within the syringe and artery. Filling the artery with the opening uppermost can assist with this. Attach the artery and syringe to the testing apparatus, making sure that there are as few air bubbles in the tubing as possible. The peristaltic pump should be calibrated so that it delivers approximately 3 ml/min. If available, the PLC should be operated according to a predetermined range of pressures and hold times as appropriate for the article being tested. If under manual control, the pressure/time profile to be followed is attained by manually turning the pump on and off while referencing the system pressure as read out by one or more pressure-reading components of the system. Following the conclusion of testing, the haemostatic dressing is subjectively assessed with regard to adhesion to the artery and formation of a ping in the artery hole. Any variations from the positive control should be noted on the data collection form.

### Success Criteria

Haemostatic dressings that are able to withstand pressures for 3 minutes are considered to have passed the assay. When a haemostatic dressing has successfully passed the assay the data collection should be stopped immediately so that the natural decrease in pressure that occurs in the artery once the test is ended isn't included on the graphs. Should the operator fail to stop data collection, these points can be deleted from the data file to avoid confusing the natural pressure decay that occurs post-test with an actual dressing failure. The entire testing period from application of the haemostatic dressing to completion must fall within pre-established criteria. The maximum pressure reached should be recorded on the data collection form.

### Failure Criteria

Haemostatic dressings that start leaking saline at any point during testing are considered to have reached the end of the assay.

NOTE: Build failures that are caused by artery swelling can be ignored and the test continued or re-started (as long as the total testing time doesn't fail beyond the established limit).

When leakage does occur, the pressure should be allowed to fall ˜20 mmHg before data collection is stopped so that die failure is easily observed on the graphs. The pressures at which leakage occurred should be recorded on the data collection form. Should the data collection stop in the middle of the experiment doe to equipment failure the data can be collected by hand at 5 second intervals until the end of the test or haemostatic dressing failure, whichever happens first The data points should be recorded on the back of the data collection form, clearly labeled, and entered by hand into the data tables.

### Exclusion Criteria

If the total testing period exceeds the maximum allowed for that procedure, regardless of cause, results must be excluded. If there are leaks from collaterals that can't be fixed either by patching or finger pressure the results must be excluded. If the test fells because of leaks at the O-rings, the results must be excluded. If the mesh support material does not completely cover the hole in the artery, the results must be excluded.

### Adherence Performance Testing

### Equipment and Supplies

Hemostat(s), Porcine artery and haemostatic dressing, optionally after performance of EVPA assay.

### Preparation of the Artery+Dressing

After application of the dressing without completion of the EVPA Assay, the dressing is ready for the Adherence Assay and Weight Limit Test (if applicable). After application of the dressing and subsequent EVPA Analysis, the artery and syringe system is then disconnected slowly from the pump so that solution does not spray everywhere. The warmed, red saline solution from the EVPA Assay remains in the syringe until the Adherence Assay and Weight Limit Test (if applicable) is completed.

### Performance of the Adherence Assay

1. After preparation of the artery and dressing (with or without EVPA analysis), gently lift the corner of the mesh and attach a hemostat of known mass to the corner.
NOTE: If the FD developed a channel leak daring the performance of the EVPA Assay, test the adherence on the opposite of the haemostatic dressing to obtain a more accurate assessment of the overall adherence.

2. Gently let go of the hemostat, taking care not to allow the hemostat to drop or twist. Turn the syringe so that the hemostat is near the top and allow the hemostat to peel back the dressing as far as the dressing will permit. This usually occurs within 10 seconds. After the hemostat has stopped pealing back the dressing, rate the adherence of toe bandage according to the following scale:

TABLE 1.1

| Dressing Performance Score | Amount of Adherence |
|---|---|
| 4 | 90+% |
| 3 | 75-90% |
| 2 | 90-75% |
| 1 | ~50% |
| 0.5 | Only the plug holds the hemostat |
| 0 | No adherence |

### Exclusion Criteria

The mesh support material must remain over the hole in the artery. If it has shifted during the polymerization and does not completely cover the hole the haemostatic dressing must be excluded.

### Success Criteria

Dressings that are given an adherence score of 3 are considered to have passed the assay.

### Failure Criteria

If a dressing does not adhere to the artery after application and/or prior to performing the EVPA assay, it is given a score of 0 and falls the adherence test. If a dressing receives a score≦2, the dressing is considered to have failed the Adherence Assay.

### Weight Held Performance Assay

After the Initial scoring of the “Adherence Test”, weights may then be added to the hemostat in an incremental manner until the mesh support material is pulled entirely off of the artery. The maximum weight that the dressing holds is then recorded as a measure of the amount of weight the dressing could hold attached to the artery.

### Example 2

Similar to the need to evaluate a test article in the context of sealing and injury deep within surrounding tissue, there was also a need to test products that can seal injured tissue where the injured vessels are smaller and thinner-walled than an aorta. The following assay accomplishes this goal.

According to this modification, the porcine carotid artery is attached to a barbed female connector using cotton thread with the connective tissue side exposed. This is in contrast to the standard EVPA where, the internal side is exposed. As the carotid arteries used in the VA model are more elastic and friable fears the aorta, it is more difficult to treat or abrade the surface without damaging and compromising the artery. To ensure that no tears have occurred during the removal of the bulk of the connective tissue, the artery is connected to the barbed connector and solution is pumped into it. If the artery is intact, a 1.5 mm hole is punched into the artery using a biopsy punch.

After the artery is prepped, it is connected to the pump system and placed on top of a piece of foam with a concave “hollow” cut into the surface. This serves as a support for the artery during application of the FD and “compression” of the artery. The test article is applied to the top of the hole and wet with 37° C. 0.9% NaCl. The artery is covered with plastic wrap, and a weight warmed to ˜38-40° C. is then placed on top of the artery. The artery is partially compressed instead of being pressed flat because of the support of the foam.

After the weight has been applied for 5 min., it is then removed, and the pump is turned on. When the solution is coming out of the end of the artery, it is then clamped and allowed to pressurize until 250 mmHg or a leak occurs, whichever comes first.

In development of the assay, the following variables were considered and tested:

Tissue Selection:

In order to mimic a vascular access procedure, a tissue substrate that was elastic yet strong was needed. Contact with rendering companies such as PelFreeze and Animal Technologies revealed 2 types of arteries collected that could be potentially used to mimic the vascular access procedure: porcine renal arteries and porcine carotid arteries. These arteries were comparable in size to a human femoral artery. Both types were purchased to examine their usefulness. The porcine renal artery was too short in useable length (less than 2″), to small an internal diameter, and not as elastic as desired. The porcine carotid artery, however, was highly elastic and offered useable segments of 3-5″ without branching or collateral arteries.

Artery Hole Size:

To determine a size to use for the assay, the actual surgical procedure was mimicked Insofar as possible. A hole was put into the artery using an 18-gauge needle. A 200 uL pipette tip was then pushed into the hole to the point where the diameter was ˜3.5 mm, just larger than a 10 F catheter. The tip was left in place for 2 hrs. and was then removed. The resulting hole was larger than the initial 18-gauge needle punch and, when compared to 2.8, 2.0, and 1.5 mm holes, was very similar to the 1.5 mm hole produced by the biopsy punch.

Surface Preparation:

In the EVPA assay, the interior surface of a porcine aorta is gently abraded using the edge of a pair of scissors to provide a “damaged” surface to which the FD would adhere, mimicking large trauma. For the vascular access procedure, obtaining a uniform, reproducible surface on which to test the FD was important. Starting with the familiar, the carotid artery was turned inside-out and abraded. However, this did not work as the carotid artery is highly elastic, and the scraping of the surface created tears that rendered the artery unusable. Using the exterior surface, the arteries that had the connective tissue carefully removed down to the level of the artery provided a surface that was uniform and best mimicked the vascular access procedure.

Integration of the Artery into the Pump System:

To best mimic the vascular access procedure, the use of the artery without any internal support to interfere with compression was desired. In order to incorporate the artery into the pump system, it was necessary to attach the artery at one end to the tubing and still have an open end to allow solution flow prior to pressurization. After examining different types of tubing and connectors, a barbed low-pressure female connector was chosen. The barb could be either 1/8″ or ¼″, depending upon the inner diameter of the carotid artery. To attach the artery to the barbed end, cable ties, o-rings, and thread were tested. Only the thread prevented leakage during pressurization.

Arterial Support:

In trying to partially-compress the artery on a flat surface, it became clear that some form of support was needed to prevent the artery from shifting during application of the FD and to prevent total compression of the artery. A variety of materials were tested, including gel packs. Styrofoam packaging material, and foam pieces. Foam pieces that had a concave trough cut into the top surface offered she best support: the trough held the artery in place, and it was cut just deep enough to allow partial compression of the artery.

Compression Method:

In the actual surgical procedure, hemostasis is more commonly achieved by manual compression of the artery for a period of ˜20 min. During this time, arterial flow is maintained. Application of a weight to the artery was tested in order to mimic this at the lab bench. Various weights in beakers just large enough to contain the weight were tested on arteries in the foam arterial support. With this set-up, both 200 g and 500 g weight inside a glass beaker (to provide a uniform surface for compression) just large enough to accommodate the weight proved to be ideal for compression. Weights lower or higher provided insufficient or too much compression, respectively.

Temperature Maintenance:

FXIII, a component of the FD that is responsible for cross-linking of fibrin monomers, is thermally labile, and the assay needs maintained around normal body and wound temperatures of 34-36° C. As this set-up cannot be easily transferred to an incubator as in the EVPA, another method had to be devised. Various methods were considered such as warmed gel packs, heating pads, and warming lamps. While these methods would produce a warmer-than-ambient temperature, they were difficult to control to the level that this assay requires. The most practical method was the use of a heat block set to 37° C. While a heat block can maintain a constant temperature for very long periods of time, they were not sufficient to warm the artery and FD to 34-36° C. in the 5 minute time frame of the assay. As the weight that is applied could be a potential heat source, it was warmed in the incubator prior to application, and this addition to the 37° C. heat block was sufficient to maintain the 34-36° C. temperature range.

Data Collection:

For this assay, fee following pieces of data are collected: amount of saline required to wet the dressing, ease of wetting, artery temperature after the incubation period, maximum pressure obtained, failure mode (channel leak, leak through plug), qualitative assessment of the adherence of the dressing to the artery, and overall comments on dressing appearance (mottled, pre-formed fibrin, thin, etc.)

### 1 Test Protocol for Ex Vivo Porcine Carotid Artery Assay (EVPCA)

Equipment and Supplies
In-line high pressure transducer (Ashcroft Duralife or equivalent)
Peristaltic pump (Pharmacia Biotech, Model P-1 or equivalent)
Voltmeter (Craftsman Professional Model 82324 or equivalent)
Computer equipped with software for recording pressure or voltage information
Tygon tubing (asst. sizes) with attachments
Water bath (Baxter Durabath or equivalent), preset to 37° C.
Heat Block (Thermolyne Type 16500 Dri-Bath or equivalent)
Incubation chamber (VWR, Model 1400G or equivalent), preset to ˜40° C.
Thermometer to monitor temperatures of water bath, heat block, and oven
Calibration weights: 200 g and 500 g
Beakers to hold calibration weights
Biopsy-punch(es), 1.5 mm or other required sizes
Assorted forceps, hemostats, and scissors
P-200 and P-100 Pipetman with tips
Plastic 1/8″ and ¼″ low pressure fittings, female connector with a barbed tubing connection
Plastic strips 1/8″ and ¼″ wide

Materials and Chemicals
Porcine carotid arteries (xxx or equivalent)
0.9% Saline, maintained at 37° C.
Red food coloring
Quilting thread or other heavy-gauge thread
Plastic Wrap
Foam pieces with a concave area cut into the surface

Preliminary Procedures

Artery Cleaning and Storage
1. Store arteries at −20° C. until used.
2. Thaw arteries at 37° C. in H2O bath.
3. Clean fat and connective tissue from exterior surface of artery.
4. The arteries may be refrozen to −20° C. and stored until use.

Artery Preparation for Assay
1. Make sure that all connective tissue is removed from the artery.
2. If any collateral arteries or other large holes are visible, cut the artery at the hole. If a small section of artery is produced from the cut, discard it. If 2 pieces are produced that are at least 1½″ long, both pieces may be used for assays.
3. Insert the barbed end of a low pressure connector, either 1/8″ or ¼″ depending upon the internal diameter of the artery, into the larger end of the artery.
4. Cut a piece of quilting or other heavy thread ˜6″ long. Using a square knot, tie the artery to the connector so that it does not come off of the connector.
   Note: As it is possible to tear the artery during the cleaning process, it is important to “leak test” the artery prior to performing the assay.
5. Connect the artery to the male connector at the end of the tubing attached to the pump system.
6. Turn on the pump with the open end of the artery pointed upwards and allow the artery to fill with the red 0.9% NaCl. When the artery is full, clamp the artery closed using a hemostat.
7. Watch the artery as it pressurizes to see if any holes or tears are present. If a hole is present, turn off the pump, unclamp the artery over a beaker to catch the saline solution, and disconnect it from the pump system.
   For arteries with Holes

If the hole is near the open end of the artery, cut off the artery at the hole, leaving the artery attached to the connector.

If the hole is near the connector, remove the artery from the connector, cut the artery at the hole, and re-attach it to the connector as outlined above.

For arteries that have pieces cut off, the remaining piece should be at least 1½″ long. If not, it should be discarded.

If the hole is near the middle of the artery, check the size of the hole. If it is less than 1.5 mm, it may be used for the assay as a hole may be punched around it. If the hole is larger than 1.5 mm, the artery should be discarded.

For Arteries without Holes
If no holes are seen, allow the artery to pressurize to ˜100 mmHg (a reading of 2.0 on the pressure gauge). Turn off the pump and unclamp the artery over a beaker, and disconnect it from the pump system.
If any holes become visible during this period, unclamp the artery over a beaker, disconnect form the pump system, and fix the artery according to the procedures outlined above.
   Note: After the artery has been inspected and any unwanted holes addressed, the test hole may then be punched in the artery
8. Insert a plastic strip into the open end of the artery so that it goes most of the way into the artery.
9. Using the biopsy punch, carefully punch a hole in the artery. Make sure that the punch connects with the plastic strip so that no additional holes are punched in the artery.
10. The punch should totally remove the center portion. If it does not, gently remove it with forceps or by re-cutting it using the biopsy punch.
11. Place the artery in the warmed, moistened container and place in the ˜40° C. incubation chamber to keep the artery moist prior to assay

### Solution and Equipment Preparation

1. Turn on the heat block and check to see that if is maintained at 37° C.
2. Check to see that the water bath is maintained at 37° C. and incubation chamber is maintained at ˜40° C.
3. Make sure that there is sufficient 0.9% saline in the pump's reservoir for completion of the day's assays. Add more if needed.
4. Place 0.9% saline into containers its a 37° C. water bath so that the solutions will be warmed prior to performing the assay.
5. The peristaltic pump should be calibrated so that it delivers approximately 3 ml/min. If not, adjust the settings at this point
6. Check the tubing for air bubbles. If bubbles exist, turn on the pump and allow the 0.9% saline to flow until all bubbles are removed.

### Application of the FD or HD

1. Place a piece of foam with the concave surface on top of the heating block and cover with a piece of plastic wrap.
2. Remove an artery from the warming box and attach it to the pump system.
3. Allow the artery to rest in the concave hollow of the foam piece.
4. Open the haemostatic dressing pouch and remove haemostatic dressing(s). Place any extras in the vacuum dessicator.
5. Place the dressing, mesh support material side UP (or the side closest to the bottom of the mold if no support material is present), over the hole in the artery
6. Slowly wet the haemostatic dressing with an amount of saline appropriate for the article being tested
   NOTE: A standard (13-15 mg/cm² of fibrinogen) 2.4×2.4 cm haemostatic dressing should be wet with 800 μl of saline or other blood substitute. A dressing of 1.5×1.5 cm would require 300 μl of saline or other blood substitute, and a 0.7×0.7 cm dressing would require 70 μl of saline or other blood substitute. The amount of saline used can be adjusted depending on the requirements of the particular experiment being performed; however, any changes should be noted on the data collection forms.
   NOTE: Wet the haemostatic dressing drop wise with 0.9% saline warmed to 37° C. or other blood substitute, taking care to keep the saline from running off the edges. Any obvious differences in wetting characteristics from the positive control should be noted on data collection forms.
7. Cover the artery with plastic wrap, taking care that the dressing doesn't slide around on the surface of the artery.
8. Place a warmed weight carefully on top of the dressing so that it does not shift off of the hole in the artery.
9. Allow the weight to remain on the artery on top of the 37° C. heat block for the duration of the polymerization time.
   NOTE: Time, pressure, and hole size can be altered according to the requirements of the experiment; changes from the standard conditions should be noted on the data collection forms.
10. After polymerization, carefully unwrap the artery and note the condition of the haemostatic dressing. Any variation front the positive control should be noted on the data collection form.
EXCLUSION CRITERION: The mesh support material must remain over the hole in the artery. If it has shifted during the polymerization and does not completely cover the hole the haemostatic dressing must be excluded.

### Testing Procedure

A diagram of testing equipment set-up is shown in FIG. 1.

TABLE 2.1
Conversion Table for Pressure (PSI) to mmHg and Voltage

| Pressure Gauge | | |
|---|---|---|
| Reading (PSI) | mm Hg Equivalent | Voltage Equivalent |
| 1.0 | 50 | 1.25 |
| 2.0 | 100 | 1.50 |
| 3.0 | 150 | 1.75 |
| 4.0 | 200 | 2.00 |
| 5.0 | 250 | 2.25 |

### Equipment and Artery Assembly

1. After the polymerization period is complete, carefully remove the plastic wrap so that the dressing is not disturbed.
2. Trim on the pump and gently Sift the open end of the artery with a hemostat. Allow the artery to fill to the top with 0.9% NaCl. This is done to minimize air bubbles in the system.
3. The system should be operated according to a pre-determined range of pressures and hold times as appropriate for the article being tested. Should the pressure drop below the desired maximum during the hold period, the pump should be turned on again until the maximum pressure is achieved.
4. Should a leak in the system develop other than failure of the FD or HD (i.e. leaking from a hole in the artery, etc.), attempts to correct the problem, should be taken. This might involve clamping the leak for the remainder of the assay. Should the attempts to fix the problem be ineffective, the test article will be excluded from analysis and called a “system failure” (See Exclusion Criteria below).
5. Following the conclusion of testing, the haemostatic dressing is subjectively assessed with regard to adhesion to the artery and formation of a plug in the artery hole. Any variations from the positive control should be noted on the data collection form.

### Success/Fail and Exclusion Criteria

Success criteria
1. Haemostatic dressings that are able to withstand various pressures for 3 minutes are considered to have passed the assay.
2. When a haemostatic dressing has successfully passed the assay the data collection should be stopped immediately so that the natural decrease in pressure that occurs in the artery once the test is coded isn't included on the graphs. Should the operator fail to stop data collection, these points can be deleted from the data file to avoid confusing the natural pressure decay feat occurs post-test with an actual dressing failure.
3. The entire testing period from application of the haemostatic dressing to completion must fell within pre-established criteria.
NOTE: For a single-step increase to maximum pressure the entire testing period should not exceed 15 minutes. Other time limits may be established for other test procedures, and should be noted on the data collection forms.
   4. The maximum pressure reached should be recorded on the data collection form.
NOTE: Typical challenge is 250 mmHg for three minutes in one step, but that may be altered based on the article being tested. The pressure, for example, may be increased in “steps” with holds at various pressures until the 256 mmHg is achieved. One example is increasing the pressure in 50 mmHg increments with a 1 minute hold at each step to ensure that the FD or HD can hold th

### Failure Criteria

1. Haemostatic dressings that start leaking saline at the point of FD or HD attachment at any point during testing are considered to have failed the assay.
NOTE: Build failures that are caused by artery swelling can be ignored and the test continued or re-started (as long as the total testing time doesn't fail beyond the established limit).
   2. When leakage from the FD or HD does occur, the pressure should be allowed to fall ˜20 mmHg before data collection is stopped so that the failure is easily observed on the graphs.
   3. The pressures at which leakage occurred should be recorded on the data collection form.
   4. Should the data collection stop in the middle of the experiment due to equipment failure the data can be collected by hand at 3 second intervals until the end of the test or haemostatic dressing failure, whichever happens first. The data points should be recorded on the hack of the data collection form, clearly labeled, and entered by hand into the data tables.

### Exclusion Criteria

1. If the total testing period exceeds the maximum allowed for that procedure, regardless of cause, results must be excluded.
2. If there are leaks from holes that can't be fixed by clamping or finger pressure the results must be excluded.
3. If the mesh support material does not completely cover the hole in the artery, the results must be excluded

### Example 3

For all dressings, ERL fibrinogen lot 3130 was formulated in CFB. The final pH of the fibrinogen was 7.4±0.1. The fibrinogen concentration was adjusted to 37.5 mg/ml. Once prepared the fibrinogen was placed on ice until use. Thrombin was formulated in CTB. The final pH of the thrombin was 7.4±0.1. The thrombin was adjusted to deliver 0.1 units/mg of Fibrinogen or 25 Units/ml thrombin. For the group with shredded support material dispersed within, it was cut into approximately 1 mm×1 mm pieces and dispersed within the thrombin solution prior to filling the molds. Once prepared the thrombin was placed on ice until use. The temperature of the fibrinogen and thrombin prior to dispensing was 4° C.±2° C. Cylindrical molds made of 10 or 3 mL polypropylene syringes (Becton Dickinson) with the luer-lock end removed were used. The plungers were withdrawn to the 6 mL and 2 mL mark respectively. For dressings utilizing a support material, the support material was cut and placed into each mold and pushed down until it was adjacent to the plunger. Once prepared the molds were placed upright and surrounded by dry ice, leaving the opening exposed at the top. 1 ml of fibrinogen and 0.15 mL of thrombin (with or without support material dispersed within) were dispensed into the 10 mL molds and 1 ml of fibrinogen and 0.15 mL of thrombin (with or without support material dispersed within), were dispensed into the 3 mL molds, which were allowed to freeze for 5 minutes. The molds were then placed into the −80° C. freezer for at least two hours before being placed into the freeze dryer and lyophylized as described above. The compositions are shown in Table 3.1 below.

TABLE 3.1

| | Fibrinogen Dose | Thrombin Dose | |
|---|---|---|---|
| Mold Size | (mg/cm²) | (U/cm²) | T:F Ratio |
| 3 ml | 75 | 7.5 | 0.1 |
| 10 ml | 25 | 2.5 | 0.1 |

Upon removal from the lyophylizer, both groups were performance tested in a modified EVPA assay as described in Example 1 above. Briefly, a plastic foam form was slipped over the artery. This covering had a hole in it that corresponded to the hole in the artery and the surrounding tissue. Warm saline was added to the surface of the dressing and the mold was immediately passed down thru the hole in the foam to the artery surface. The plunger was then depressed and held by hand for 3 minutes, after which the mold was withdrawn as the plunger was depressed further. At this point the artery was pressurized and the assay continued as described in Example 1 above.

Results

TABLE 3.1

| | Mold | EVPA Result | Maximum |
|---|---|---|---|
| Support Material | Size | (@250 mmHg) | Pressure |
| None | 10 ml | Pass | >250 mmHg |
| Dexon Mesh Backing | 10 ml | Pass | ″ |
| ″ | 3 ml | Pass | ″ |
| Shredded Dexon Mesh (Dispersed) | 10 ml | Pass | ″ |
| ″ | 3 ml | Fail | 150 mm Hg |

Conclusions:

Dressings that included no support material or a DEXON™ mesh support material performed well, with all passing the EVPA test at 250 mmHg. When the support material was dispersed throughout the composition, the dressings also performed well, with the large size (10 mL mold) dressings holding the fall 250 mmHg of pressure, while the smaller held up to 150 mmHg of pressure. This indicates that the use of a support material may be optional, and it's location may be on the ‘back’ of the dressing, or dispersed throughout the composition, as desired.

The results demonstrate that the dressings were effective at the highest pressure tested regardless of size, and that they functioned effectively regardless of the presence or absence of the support material. Higher performance was associated with the presence of support material, and a larger applicator.

### Example 4

Dexon™ Mesh support material was cut to fit into and placed into each PETG 1.5×1.5 cm mold. Fifteen microliters of 2% sucrose was pipeted on top of each of the four corners of the support material and the molds were placed inside a −80° C. freezer. Once completed the molds were placed in a −80° C. freezer. All molds remained in the −80° C. freezer for at least 60 minutes. Enzyme Research Laboratories (EEL) Fibrinogen lot 3150 was formulated in 100 mM Sodium Chloride, 1.1 mM Calcium Chloride, 10 mM Tris, 10 mM Sodium Citrate, and 1.5% Sucrose (Fibrinogen complete buffer). In addition, Human Serum Albumin was added to 80 mg/g of total protein and Tween 80 (non-animal source) was added to 15 mg/g total protein. The final pH of the fibrinogen was 7.4+/−0.1. The fibrinogen concentration was adjusted to 37.5 mg/ml. Once prepared the fibrinogen was placed on ice until use. Thrombin was formulated in 150 mM Sodium Chloride, 40 mM Calcium Chloride, 10 mM Tris and 100 mM L-Lysine. The final pH of the thrombin was 7.4+/−0.1. The thrombin was adjusted to 25 Units/ml thrombin, resulting in 0.1 units/mg of Fibrinogen or 1.3 U/cm². Once prepared the thrombin was placed on ice until use. The temperature of the fibrinogen and thrombin prior to dispensing was 4° C.+/−2° C. Molds were removed from the −80° C. freezer and placed on a copper plate that was placed on top of dry ice. A repeat pipetor was filled with fibrinogen and second repeat pipetor was filled with thrombin. Simultaneously 0.8 ml of fibrinogen and 133 micro liters of thrombin were dispensed into each mold. Once the molds were filled, they were returned to the −80° C. freezer for at least two hours before being placed into a pre-cooled Genesis™ lyophylizer (Virtis, Gardiner, N.Y.). The chamber was sealed and the temperature equilibrated. The chamber was then evacuated and the dressings lyophilized as described in Example 3.

Test articles of a different size were also prepared as follows. Support material was cut and placed into each PETG 0.7×0.7 cm mold. Five microliters of 2% sucrose was pipeted on top of each of the four comers of the support material and the molds were placed inside a −80° C. freezer. Once completed the molds were placed in a −80° C. freezer. All molds remained in the −80° C. freezer for at least 60 minutes. Enzyme Research Laboratories (ERL) Fibrinogen lot 3150 was formulated in 100 mM Sodium Chloride, 1.1 mM Calcium Chloride, 10 mM Tris, 10 mM Sodium Citrate, and 1.5% Sucrose (Fibrinogen complete buffer). In addition, Human Serum Albumin was added to 80 mg/g of total protein and Tween 80 (non-animal source) was added to 15 mg/g total protein. The final pH of the fibrinogen was 7.4+/−0.1. The fibrinogen concentration was adjusted to 39.2 mg/ml. Once prepared the fibrinogen was placed on ice until use. Thrombin was formulated in 150 mM Sodium Chloride, 40 mM Calcium Chloride, 10 mM Tris and 100 mM L-Lysine. The final pH of the thrombin was 7.4+/−0.1. The thrombin was adjusted to 25 Units/ml thrombin, which resulted in a final composition of 0.1 units/mg of Fibrinogen or 1.3 U thrombin/cm². Once prepared the thrombin was placed on ice until use. Use temperature of the fibrinogen and thrombin prior to dispensing was 4° C.+/−2° C. Molds were removed from the −80° C. freezer and placed on a copper plate that was placed on top of dry ice. A repeat pipetor was filled with fibrinogen and second repeat pipetor was filled with thrombin. Simultaneously 0.17 ml of fibrinogen and 26 micro liters of thrombin were dispensed into each mold. Once the molds were filled, they were returned to the −80° C. freezer for at least two hours before being placed into the freeze dryer and lyophylized as described above.

The performance of the test articles was determined using the EVPCA assay as described in Example 2 above.

Results:

TABLE 4.1

| Test | Fibrinogen | Thrombin | | % | % | % | % |
|---|---|---|---|---|---|---|---|
| Article | Dose | Dose | T:F | Reaching | Reaching | Reaching | Reaching |
| Size (cm²) | (mg/cm²) | (U/cm²) | (U/mg) | 100 mmHg | 150 mmHg | 200 mmHg | 250 mmHg |
| 0.7 | 13 | 1.3 | 0.1 | 100 | 80 | 40 | 40 |
| 1.5 | 13 | 1.3 | 0.1 | 100 | 80 | 80 | 60 |

### Example 5

Dexon™ Mesh support material was cut to fit into and placed into each PETG 1.5×1.5 cm mold. Fifteen microliters of 2% sucrose was pipeted on top of each of the four corners of the support material and the molds were placed inside a −80° C. freezer. PETG 1.5×1.5 cm molds that did not contain support material were also placed inside the −80° C. freezer. In a third group, the same amount of support material was cut into small pieces (approximately less than 2 mm×2 mm) and placed into PETG 1.5×1.5 cm molds (these dressings are referred to as having their support material ‘dispersed’). Once completed the molds were placed in a −80° C. freezer. All molds remained in the −80° C. freezer for at least 60 minutes. Enzyme Research Laboratories (ERL) Fibrinogen lot 3130 was formulated in 100 mM Sodium Chloride, 1.1 mM Calcium Chloride, 10 mM Tris, 10 mM Sodium Citrate, and 13% Sucrose (Fibrinogen complete buffer). In addition, Human Serum Albumin was added to 80 mg/g of total protein and Tween 80 (non-animal source) was added to 15 mg/g total protein. The final pH of the fibrinogen was 7.4+/−0.1. The fibrinogen concentration was adjusted to 36.56 mg/ml and 14.06 mg/ml. Once prepared the fibrinogen was placed on ice until use. Thrombin was formulated in 150 mM Sodium Chloride, 40 mM Calcium Chloride, 10 mM Tris and 100 mM L-Lysine. The final pH of the thrombin was 7.4+/−0.1. The thrombin was adjusted to deliver 0.01, 0.1 or 1 units/mg of Fibrinogen or 2.5, 25 or 250 Units/ml thrombin. Once prepared the thrombin was placed on ice until use. The temperature of the fibrinogen and thrombin prior to dispensing was 4° C.+/−2° C. Molds were removed from the −80° C. freezer and placed on a copper plate that was placed on top of dry ice. A repeat pipetor was filled with fibrinogen and second repeat pipetor was filled with thrombin. Simultaneously 0.8 ml of fibrinogen and 13.3 micro liters of thrombin were dispensed into each mold. Once the molds were filled, they were returned to the −80° C. freezer for at least two hours before being placed into the freeze dryer. Table 5.1 shows the experimental design.

TABLE 5.1
Experimental Design

| Fibrinogen Dose | Thrombin Dose | T:F | Support |
|---|---|---|---|
| (mg/cm²) | (U/cm²) | (U/mg) | material |
| 5 | 0.05 | 0.01 | Yes |
| 5 | 0.05 | 0.01 | No |
| 5 | 0.05 | 0.01 | Dispersed |
| 5 | 0.5 | 0.1 | Yes |
| 5 | 0.5 | 0.1 | No |
| 5 | 0.5 | 0.1 | Dispersed |
| 5 | 5 | 1 | Yes |
| 5 | 5 | 1 | No |
| 5 | 5 | 1 | Dispersed |
| 13 | 0.13 | 0.01 | Yes |
| 13 | 0.13 | 0.01 | No |
| 13 | 0.13 | 0.01 | Dispersed |
| 13 | 1.3 | 0.1 | Yes |
| 13 | 1.3 | 0.1 | No |
| 13 | 1.3 | 0.1 | Dispersed |
| 13 | 13 | 1 | Yes |
| 13 | 13 | 1 | No |
| 13 | 13 | 1 | Dispersed |

The performance of the test articles was determined using the EVPCA assay as described in Example 2 above.

Results:

TABLE 5.2

| Fibrinogen | Thrombin | Thrombin | | % | % | % |
|---|---|---|---|---|---|---|
| Dose | Dose | (U/mg | Support | Reaching | Reaching | Reaching |
| (mg/cm²) | (U/cm²) | fibrinogen) | material | 150 mmHg | 200 mmHg | 250 mmHg |
| 5 | 0.05 | 0.01 | Yes | 66.6 | 50 | 50 |
| 5 | 0.05 | 0.01 | No | 0 | 16 | 0 |
| 5 | 0.05 | 0.01 | Dispersed | 13.3 | 0 | 0 |
| 5 | 0.5 | 0.1 | Yes | 66.6 | 66 | 50 |
| 5 | 0.5 | 0.1 | No | 60 | 20 | 0 |
| 5 | 0.5 | 0.1 | Dispersed | 40 | 0 | 0 |
| 5 | 5 | 1 | Yes | 33.3 | 0 | 0 |
| 5 | 5 | 1 | No | 0 | 0 | 0 |
| 5 | 5 | 1 | Dispersed | 0 | 0 | 0 |
| 13 | 0.13 | 0.01 | Yes | 100 | 50 | 33.3 |
| 13 | 0.13 | 0.01 | No | 33.3 | 0 | 0 |
| 13 | 0.13 | 0.01 | Dispersed | 20 | 20 | 0 |
| 13 | 1.3 | 0.1 | Yes | 66.6 | 50 | 16.6 |
| 13 | 1.3 | 0.1 | No | 0 | 0 | 0 |
| 13 | 1.3 | 0.1 | Dispersed | 100 | 80 | 40 |
| 13 | 13 | 1 | Yes | 33.3 | 33.3 | 33.3 |
| 13 | 13 | 1 | No | 33.3 | 0 | 0 |
| 13 | 13 | 1 | Dispersed | 33.3 | 16.6 | 16.6 |

### Example 6

Dexon™ Mesh support material was cut to fit into and placed into each PETG 0.7×0.7 cm mold. Five microliters of 2% sucrose was pipeted on top of each of the four corners of the support material and the molds were placed inside a −80° C. freezer. Once completed the molds were placed in a −80° C. freezer. All molds remained in the −80° C. freezer for at least 60 minutes. Enzyme Research Laboratories (ERL) Fibrinogen lot 3130 was formulated in 100 mM Sodium Chloride, 1.1 mM Calcium Chloride, 10 mM Tris, 10 mM Sodium Citrate, and 1.5% Sucrose (Fibrinogen complete buffer). In addition, Human Serum Albumin was added to 80 mg/g of total protein and Tween 80 (non-animal source) was added to 15 mg/g total protein. The final pH of the fibrinogen was 7.4+/−0.1. The fibrinogen concentration was adjusted to 39.2 mg/ml and 32.06 mg/ml. Once prepared the fibrinogen was placed on ice until use. Thrombin was formulated in 150 mM Sodium Chloride, 40 mM Calcium Chloride, 10 mM Tris and 100 mM L-Lysine. The final pH of the thrombin was 7.4+/−0.1. The thrombin was adjusted to deliver 1 unit/mg of Fibrinogen or 250 Units/ml thrombin. Once prepared the thrombin was placed on ice until use. The temperature of the fibrinogen and thrombin prior to dispensing was 4° C.+/−2° C. Molds were removed from the −80° C. freezer and placed on a copper plate that was placed on top of dry ice. A repeat pipetor was filled with fibrinogen and second repeat pipetor was filled with thrombin. Simultaneously 0.2 ml of fibrinogen and 33 micro liters of thrombin were dispensed into each mold. Once the molds were filled, they were returned to the −80° C. freezer for at least two hours before being placed into the freeze dryer. Table 6.1 shows the experimental design.

TABLE 6.1
Experimental Design

| Fibrinogen Dose | Thrombin Dose | T:F | Support |
|---|---|---|---|
| (mg/cm²) | (U/cm²) | (U/mg) | material |
| 16 | 16.1 | 1 | Yes |
| 13 | 13.0 | 1 | Yes |

The performance of the test articles was determined using the EVPCA assay as described in Example 2 above.

Results:

TABLE 6.2

| | % Pass | % Pass | % Pass |
|---|---|---|---|
| Fibrinogen Dose | EVCPA at | EVCPA at | EVCPA at |
| (mg/cm²) | 150 mm Hg | 200 mm Hg | 250 mm Hg |
| 16 | 66.6 | 33.3 | 16.6 |
| 13 | 0 | 0 | 0 |

### Example 7

Dexon™ Mesh support material was cut to fit into and placed into each PETG 1.5×1.5 cm mold. Fifteen microliters of 2% sucrose was pipetted on top of each of the four corners of the support material and the molds were placed inside a −80° C. freezer. Once completed the molds were placed in a −80° C. freezer. All molds remained in the −80° C. freezer for at least 60 minutes. Enzyme Research Laboratories (ERL) Fibrinogen lot 3170P was formulated in 100 mM Sodium Chloride, 1.1 mM Calcium Chloride, 10 mM Tris, 10 mM Sodium Citrate, and 1.5% Sucrose (Fibrinogen complete buffer). In addition, Human Serum Albumin was added to 80 mg/g of total protein and Tween 80 (non-animal source) was added to 15 mg/g total protein. The final pH of the fibrinogen was 7.4+/−0.1. The fibrinogen concentration was adjusted to 36.56 mg/ml and 14.066 mg/ml. Once prepared the fibrinogen was placed on ice until use. Thrombin was formulated in 150 mM Sodium Chloride, 40 mM Calcium Chloride, 10 mM Tris and 100 mM L-Lysine. The final pH of the thrombin was 7.4+/−0.1. The thrombin was adjusted to deliver 0.001, or 0.0001 units/mg of Fibrinogen or 0.25, 0.025 Units/ml thrombin. Once prepared the thrombin was placed on ice until use. The temperature of the fibrinogen and thrombin prior to dispensing was 4° C.+/−−2° C. Molds were removed from the −80° C. freezer and placed on a copper plate that was placed on top of dry ice. A repeat pipetor was filled with fibrinogen and second repeat pipetor was filled with thrombin. Simultaneously 0.8 ml of fibrinogen and 133 micro liters of thrombin were dispensed into each mold. Once the molds were filled, they were returned to the −80° C. freezer for at least two hours before being placed into the freeze dryer. Table 7.1 shows the experimental design.

TABLE 7.1
Experimental Design

| Fibrinogen Dose | Thrombin Dose | T:F | Support |
|---|---|---|---|
| (mg/cm2) | (U/cm²) | (U/mg) | material |
| 13 | 0.013 | 0.001 | Yes |
| 13 | 0.0013 | 0.0001 | Yes |
| 5 | 0.005 | 0.001 | Yes |
| 5 | 0.0005 | 0.0001 | Yes |

The performance of the test articles was determined using the EVPCA assay as described in Example 2 above.

### Example 8

Previously manufactured lyophylized mixtures of fibrinogen & thrombin (lot #012408) were placed into a grinder (Krups) and ground (5 seconds) into a powder. The powdered dressings were placed into a 50 ml conical centrifuge tube. Twenty-five grams of sucrose was ground into powder and placed into another 50 ml conical centrifuge tube. Table 8.1 shows the design of the experiment.

TABLE 8.1
Experimental Design

| | | | | Weight of | |
|---|---|---|---|---|---|
| Fibrinogen | Thrombin | | Support | Dressing | Weight of |
| Dose | Dose | T:F | material/ | Powder | Sucrose |
| (mg/cm²) | (U/cm²) | (U/mg) | Placement | (g) | (g) |
| 5 | 1.3 | 0.26 | Yes | 0.008 | 0.092 |
| 13 | 1.3 | 0.1 | Yes/Bottom | 0.208 | 0.792 |
| 13 | 1.3 | 0.1 | Yes/Top | 0.208 | 0.792 |
| 26 | 1.3 | 0.05 | Yes/Bottom | 0.416 | 0.584 |
| 26 | 1.3 | 0.05 | Yes/Top | 0.416 | 0.584 |
| 26 | 1.3 | 0.05 | Yes/Middle | 0.416 | 0.584 |
| 56 | 1.3 | 0.023 | Yes/Top | 0.12 | 0.00 |
| 56 | 1.3 | 0.023 | Yes/Middle | 0.12 | 0.00 |
| 450 | 1.3 | 0.003 | Yes/Middle | 0.95 | 0.00 |
| 5 | 1.3 | 0.26 | No | 0.008 | 0.092 |
| 13 | 1.3 | 0.1 | No | 0.208 | 0.792 |
| 26 | 1.3 | 0.05 | No | 0.416 | 0.584 |
| 56 | 1.3 | 0.023 | No | 0.12 | 0.00 |
| 5 | 1.3 | 0.26 | Dispersed | 0.008 | 0.092 |
| 13 | 1.3 | 0.1 | Dispersed | 0.208 | 0.792 |
| 26 | 1.3 | 0.05 | Dispersed | 0.416 | 0.584 |
| 56 | 1.3 | 0.023 | Dispersed | 0.12 | 0.00 |

For each group 0.1 g of the powder/sucrose was weighed and placed into a Carver 13 mm Evacuable Pellet Die. For pellets that had a support material, 75 mg of the support material was placed in one of four locations. In the first, the support material (Dexon™ mesh) was placed into the die, followed by fee addition of the powder, these are referred to as being in the ‘bottom’ position. When the powder was placed into the die followed by the support material (Dexon™ mesh) these are referred to as in the ‘top’ position. For pellets with the support material in the ‘middle’ position, 50 mg of the powder/sucrose was weighed and placed into a Carver 13 mm Evacuable Pellet Die, followed by the support material (75 mg of Dexon™ mesh) which was then topped off by another 50 mg of the powder/sucrose mixture. For pellets that had dispersed support material, the powder and 75 mg of shredded support material (Dexon™ mesh) were added to the die at the same time and mixed for 5 seconds with a pipette tip. Once the die was filled with the appropriate material, it was placed in a Carver 4350 manual pellet press. Pressure was applied to give an applied load of 1000 lbs. The resulting pellets were removed and placed into a desiccator until tested.

The performance of the test articles was determined using the EVPCA assay as described in Example 2 above. The test articles containing a fibrinogen dose of 26 or 56 mg/cm² exhibited the best results.

### Example 9

Previously manufactured lyophylized mixtures of fibrinogen & thrombin were placed into a grinder (Krups) and ground (5 seconds) into a powder. The powdered dressings were placed into a 50 ml conical centrifuge tube. Twenty-five grams of sucrose was ground into powder and placed into another 50 ml conical centrifuge tube. Cylindrical molds made of 3 mL polypropylene syringes (Becton Dickinson) with the luer-lock end removed were used. The plungers were withdrawn to the 2 or 3 ml mark.

TABLE 9.1
Experimental Design

| | | | | Weight of | |
|---|---|---|---|---|---|
| Fibrinogen | Thrombin | | | Dressing | Weight of |
| Dose | Dose | T:F | Support | Powder | Sucrose |
| (mg/cm²) | (U/cm²) | (U/mg) | material | (g) | (g) |
| 26 | 1.3 | 0.05 | No | .0208 | 0.0792 |
| 26 | 1.3 | 0.05 | Yes | .0208 | 0.0792 |
| 26 | 1.3 | 0.05 | Dispersed | .0208 | 0.0792 |
| 56 | 1.3 | 0.023 | No | 0.0448 | 0.0552 |
| 56 | 1.3 | 0.023 | Yes | 0.0448 | 0.0552 |
| 56 | 1.3 | 0.023 | Dispersed | 0.0448 | 0.0552 |
| 150 | 1.3 | 0.0087 | No | 0.12 | 0.0 |
| 150 | 1.3 | 0.0087 | Yes | 0.12 | 0.0 |
| 150 | 1.3 | 0.0087 | Dispersed | 0.12 | 0.0 |
| 450 | 1.3 | 0.003 | No | 0.36 | 0.0 |
| 450 | 1.3 | 0.003 | Yes | 0.36 | 0.0 |
| 450 | 1.3 | 0.003 | Dispersed | 0.36 | 0.0 |

For dressings utilizing a support material, 75 mg of Dexon mesh support material was cut to fit into the mold and then placed into each mold, and pushed down until it was adjacent to the plunger. Where syringes had dispersed support material, an equivalent amount of support material was shredded and dispersed within the powder that was added to each syringe. For each group 0.1 g of the powder/sucrose was weighed and placed into a each syringe, except for the 150 mg/cm² group which had 0.12 g added to the syringe. Gelfoam™ was cut to fit into the mold and then placed inside the syringes, either alone or with 26 mg/cm² of dressing powder.

The performance of the test articles was determined using the EVPCA assay as described in Example 2 above. The results are shown in Table 9.2 below.

Results:

TABLE 9.2

| Fibrinogen | | % | % | % | % | % |
|---|---|---|---|---|---|---|
| Dose | Support | Reaching | Reaching | Reaching | Reaching | Reaching |
| (mg/cm²) | Material | 60 mmHg | 100 mmHg | 150 mmHg | 200 mmHg | 250 mmHg |
| 26 | Yes | 100 | 100 | 0 | 0 | 0 |
| 26 | Dispersed | 0 | 0 | 0 | 0 | 0 |
| 26 | No | 0 | 0 | 0 | 0 | 0 |
| 26 | Gelfoam ™ | 100 | 0 | 0 | 0 | 0 |
| 56 | Yes | 100 | 100 | 100 | 100 | 100 |
| 56 | Dispersed | 100 | 100 | 100 | 100 | 100 |
| 56 | No | 0 | 0 | 0 | 0 | 0 |
| 150 | Yes | 100 | 100 | 100 | 100 | 50 |
| 150 | Dispersed | 100 | 100 | 100 | 50 | 50 |
| 150 | No | 100 | 100 | 100 | 100 | 100 |
| 450 | Yes | 100 | 100 | 100 | 100 | 0 |
| 450 | Dispersed | 100 | 100 | 100 | 100 | 100 |
| 450 | No | 100 | 100 | 100 | 0 | 0 |

### Example 10

Enzyme Research Laboratories (ERL) Fibrinogen lot 3170P was formulated in 100 mM Sodium Chloride, 1.1 mM Calcium Chloride, 10 mM Tris, 10 mM Sodium Citrate, and 1.5% Sucrose (Fibrinogen complete buffer). In addition, Human Serum Albumin was added to 80 mg/g of total protein and Tween 80 (non-animal source) was added to 15 mg/g total protein. The final pH of the fibrinogen was 7.4+/−0.1. The fibrinogen concentration was adjusted to 37.5 mg/ml. Once prepared the fibrinogen was placed on ice until use. Fibrinogen was diluted to 18.75 mg/ml and 9.4 mg/ml with Fibrinogen complete buffer.

Enzyme Research Laboratories (ERL) fibrinogen lot 3170P was formulated in 100 mM Sodium Chloride, 1.1 mM Calcium Chloride, 10 mM Tris, 10 mM Sodium Citrate, and 1.5% Sucrose (Fibrinogen complete buffer). This group did not contain Sucrose or Tween. The final pH of the fibrinogen was 7.4+/−0.1. The fibrinogen concentration was adjusted to 37.5 mg/ml. Once prepared the fibrinogen was placed on ice until use. Thrombin-was formulated in 150 mM Sodium Chloride, 40 mM Calcium Chloride, 10 mM Tris and 100 mM L-Lysine. The final pH of the thrombin was 7.4+/−0.1. The thrombin was adjusted to deliver 0.1 units/mg of Fibrinogen or 25 Units/ml thrombin. Thrombin was diluted to 12.5 U/ml and 6.25 U/ml with Thrombin buffer. Once prepared the thrombin was placed on ice until use. The temperature of the fibrinogen and thrombin prior to dispensing was 4° C.+/−2° c. Microcentrifuge tubes (0.65 ml) were placed on dry ice. There were two groups of frozen plugs prepared with one frozen plug per group. One group did not have any support material, and the second group contained shredded support material (Dexon mesh) (0.1 g) dispersed within it. A repeat pipetor was filled with fibrinogen and second repeat pipetor was filled with thrombin. Simultaneously 0.5 ml of fibrinogen and 75 micro liters of thrombin were dispensed into each microcentrifuge tube. Once each microcentrifuge tube was filled, they were transferred to a −80° C. freezer until tested. Table 10.1 shows the experimental design.

TABLE 10.1
Experimental Design

| Fibrinogen Dose | Thrombin Dose | T:F | |
|---|---|---|---|
| (mg/Item) | U/Item | U/mg | Support Material |
| 18.75 | 1.875 | 0.1 | Dispersed |
| No Sucrose | | | |
| or Tween | | | |
| 18.75 | 1.875 | 0.1 | No |
| No Sucrose | | | |
| or Tween | | | |
| 18.75 | 1.875 | 0.1 | Dispersed |
| 18.75 | 1.875 | 0.1 | No |
| 9.4 | 0.94 | 0.1 | Dispersed |
| 9.4 | 0.94 | 0.1 | No |
| 4.7 | 0.47 | 0.1 | Dispersed |
| 4.7 | 0.47 | 0.1 | No |

The performance of the test articles was determined using a modified EVPCA assay. The EVPCA assay (described in Example 2 above) was modified to further enhance the faithfulness of the assay to the actual conditions mat may be encountered in vivo. As described in Example 3, the surrounding of the test blood vessel by closely fitting material can replicate the use of these inventions in sealing an injury deep inside tissue. To further enhance this replication of such a clinical setting, tissue was substituted for the plastic foam that was wrapped around the vessel. The tissue may be chosen to best replicate the intended anatomical location. In this Example commercial meat was used to simulate the leg muscle of a patient undergoing a vascular access procedure. Sufficient tissue was used to simulate a depth of several inches of muscle tissue. Other than this modification, and the employment of an application device as described in Example #3, the assay was carried out as described in Example #2. The results are shown in Table 10.2 below

Results:

TABLE 10.2

| Fibrinogen | Thrombin | | | % | % | % | % |
|---|---|---|---|---|---|---|---|
| Dose | Dose | T:F | Support | Reaching | Reaching | Reaching | Reaching |
| (mg/Item) | (U/Item) | U/mg | Material | 100 mmHg | 150 mmHg | 200 mmHg | 250 mmHg |
| 18.75 No | 1.875 | 0.1 | Dispersed | 50 | 50 | 50 | 50 |
| Sucrose or | | | | | | | |
| Tween | | | | | | | |
| 18.75 No | 1.875 | 0.1 | No | 50 | 0 | 0 | 0 |
| Sucrose or | | | | | | | |
| Tween | | | | | | | |
| 18.75 | 1.875 | 0.1 | Dispersed | 100 | 100 | 100 | 0 |
| 18.75 | 1.875 | 0.1 | No | 100 | 100 | 100 | 0 |
| 9.4 | 0.94 | 0.1 | Dispersed | 100 | 0 | 0 | 0 |
| 9.4 | 0.94 | 0.1 | No | 0 | 0 | 0 | 0 |
| 4.7 | 0.47 | 0.1 | Dispersed | 0 | 0 | 0 | 0 |
| 4.7 | 0.47 | 0.1 | No | 0 | 0 | 0 | 0 |

### Example 11

Previously manufactured lyophylized mixtures of fibrinogen & thrombin (lot #012408) were placed into a grinder (Krups) and ground (5 seconds) into a powder. The powdered dressings were placed into a 30 ml conical centrifuge lube. Twenty-five grams of sucrose was ground into powder and placed into another 50 ml conical centrifuge tube. Table 11.1 shows the design of the experiment.

TABLE 11.1
Experimental Design

| | | | | | Weight | |
|---|---|---|---|---|---|---|
| | | | | | of | |
| Fibrinogen | Thrombin | | Support | | Dressing | |
| Dose | Dose | T:F | material and | Disk | Powder | Weight of |
| (mg/cm²) | (U/cm²) | (U/mg) | placement | Modification | (g) | Sucrose (g) |
| 26 | 1.3 | 0.05 | Yes/Dispersed | Hole in | 0.416 | 0.584 |
| | | | | center of | | |
| | | | | Disk | | |
| 26 | 1.3 | 0.05 | Yes/Dispersed | 12.5% | 0.416 | 0.584 |
| | | | | Removed as | | |
| | | | | a pie shaped | | |
| | | | | wedge | | |

For each group 0.1 g of the powder/sucrose was weighed and placed into a Carver 13 mm Evacuable Pellet Die. Seventy-five mg of shredded support material (Dexon™ mesh) was added to the powder and mixed for 5 seconds with a pipette tip. Once the die was filled with the appropriate material, it was placed in a Carver 4350 manual pellet press. Pressure was applied to give an applied load of 1000 lbs. Once the pellets were removed from the die a small hole was placed in the center of two of pellets using a 1/64″ drill bit. The other two pellets had 1/8″ of the pellet removed in a wedge-shaped piece with the vertex at the center of the pellet. The resulting pellets were removed and placed into a desiccator until tested.

The performance of the test articles was determined using the EVPCA assay as described in Example 2 above. With a modification that a 22 gauge wire was placed into the artery hole and the test article was slid down the wire to come in contact with the artery hole. Once the test article was delivered to the hole the wire was removed and the test proceeded as described. The results are shown in Table 11.2 below.

Results:

TABLE 11.2

| Fibrinogen | Support | | % | % | % | % |
|---|---|---|---|---|---|---|
| Dose | Material | Disk | Reaching | Reaching | Reaching | Reaching |
| (mg/cm²) | Placement | Modification | 100 mmHg | 150 mmHg | 200 mmHg | 250 mmHg |
| 26 | Dispersed | Hole in | 50 | 50 | 50 | 50 |
| | | center of | | | | |
| | | Disk | | | | |
| 26 | Dispersed | 12.5% | 50 | 0 | 0 | 0 |
| | | Removed as | | | | |
| | | a pie shaped | | | | |
| | | wedge | | | | |

### Example 12

Enzyme Research Laboratories (ERL) Fibrinogen lot 3107P was formulated in 100 mM Sodium Chloride, 1.1 mM Calcium Chloride, 10 mM Tris, 10 mM Sodium Citrate, and 1.5% Sucrose (Fibrinogen complete buffer). In addition, Human Serum Albumin was added to 80 mg/g of total protein and Tween 80 (non-animal source) was added to 15 mg/g total protein. The final pH of the fibrinogen was 7.4+/−0.1. The fibrinogen concentration was adjusted to 37.5 mg/ml. Once prepared the fibrinogen was placed on ice until use. Thrombin was formulated in 150 mM Sodium Chloride, 40 mM Calcium Chloride, 10 mM Tris and 100 mM L-Lysine. The final pH of the thrombin was 7.4+/−0.1. The thrombin was adjusted to deliver 0.1 units/mg of Fibrinogen or 25 Units/ml thrombin. Once prepared the thrombin was placed on ice until use. The temperature of the fibrinogen and thrombin prior to dispensing was 4° C.+/−2° C.

Cylindrical molds made of 3 mL polypropylene syringes (Becton Dickinson) with the luer-lock end removed were used. The plungers were withdrawn to the 1.0 ml mark.

Cylindrical molds were placed on dry ice. There were two groups of cylindrical molds prepared with one cylindrical mold per group. One group did not have any support material, and the second group contained shredded support material (0.1 gm Dexon™ mesh) dispersed within it. A repeat pipetor was filled with fibrinogen and second repeat pipetor was filled with thrombin. Simultaneously 0.5 ml of fibrinogen and 75 micro liters of thrombin were dispensed into each cylindrical mold. Once each cylindrical mold was filled, they were transferred to a −80° C. freezer until tested. Table 12.1 shows the experimental design.

TABLE 12.1

| Fibrinogen | Thrombin | T:F | Support |
|---|---|---|---|
| Dose (mg) | (Units) | (U/mg) | Material |
| 18.75 | 1.875 | 0.1 | Dispersed |
| 18.75 | 1.875 | 0.1 | No |

The performance of the test articles was determined using a modified EVPCA assay. The EVPCA assay (described in Example 2 above) was modified to further enhance the faithfulness of the assay to the actual conditions that may be encountered is two. As described in Example 3, the surrounding of the test blood vessel by closely fitting material can replicate the use of these inventions in sealing an injury deep inside tissue. To further enhance this replication of such it clinical setting, tissue was substituted for the plastic foam that was wrapped around the vessel. The tissue may be chosen to best replicate the intended anatomical location. In this Example commercial meat was used to simulate the leg muscle of a patient undergoing a vascular access procedure. Sufficient tissue was used to simulate a depth of several inches of muscle tissue. Other than this modification, and the employment of an application device as described in Example #3, the assay was carried out as described in Example #2. The results are shown in table 12.2

Results:

TABLE 12.2

| | | | | % | % | % | % |
|---|---|---|---|---|---|---|---|
| Fibrinogen | Thrombin | T:F | Support | Reaching | Reaching | Reaching | Reaching |
| Dose (mg) | (Units) | U/mg | Material | 100 mmHg | 150 mmHg | 200 mmHg | 250 mmHg |
| 18.75 | 1.875 | 0.1 | Dispersed | 100 | 100 | 50 | 0 |
| 18.75 | 1.875 | 0.1 | No | 0 | 0 | 0 | 0 |

### Example 13

Hemostatic test materials were manufactured using serological pipettes (with the tapered ends cut off) and absorbable PGA biofelt materials produced by Concordia Medical. Both thin (100 mg/cc) and thick (250 mg/cc) biofelts were used and were sewn into the shape of small end caps that fit onto the ends of 2 ml and 5 ml serological pipettes, respectively.

ERL fibrinogen was formulated in CFB and adjusted to a final fibrinogen concentration of 37.5 mg/ml with a pH of 7.4±0.1. Thrombin (manufactured in-house) was formulated in CTB and adjusted to a final thrombin concentration of 0.1 units/mg of fibrinogen or 25 Units/ml thrombin, with a final pH of 7.4±0.1. Once prepared, the final fibrinogen and thrombin solutions were placed on ice and cooled to 4° C.±2° C.

The 2 ml pipette applicators (capped with the thin biofelt), were manufactured by adding 0.39 ml of fibrinogen (at 4° C.±2° C.) and 0.0585 ml of thrombin (at 4° C.±2° C.) to a 5 ml round-bottom polypropylene tube (12 mm×75 mm). The contents of each tube were mixed by hand and the biofelt-covered end of the 2 ml applicator was inserted into the tube and allowed to absorb the fibrinogen and thrombin mixture for 20 seconds. The applicator was then removed and transferred into a clean 5 ml round-bottom tube which was immediately immersed in liquid nitrogen and frozen for 2 minutes. The frozen 2 ml pipette applicators were then placed at −80° C. until lyophilization.

The 5 ml pipette applicators (capped with the thick biofelt), were manufactured by adding 2.73 ml of fibrinogen (at 4° C.±2° C.) and 0.4095 ml of thrombin (at 4° C.±2° C.) to the barrel of a 10 ml syringe (plunger removed). The contents of each syringe were mixed by band and the biofelt-covered end of the 5 ml pipette kittner was inserted into the syringe and allowed to absorb the fibrinogen and thrombin mixture for 20 seconds. The applicator was then removed and transferred into a clean 10 ml syringe which was immediately immersed in liquid nitrogen and frozen for 2 minutes. The frozen 5 ml pipette applicators were then placed at −80° C. until lyophilization.

The same fibrinogen and thrombin solutions used to manufacture the pipette kittners were also used to manufacture FDs (1.5 cm×1.5 cm) with both biofelts as well as DEXON™ as backing materials. These FDs were tested in the EVPA and Adherence assays and all passed 100%, with adherence scores of 4.0.

Hemostatic test materials were manufactured using absorbable PGA biofelt materials produced by Concordia Medical. Both thin (100 mg/cc) and thick (250 mg/cc) biofelts were out into 2, 3, or 5 mm diameter discs. The biofelt discs were then attached onto the ends of serological pipettes of a similar diameter (with the tapered ends cut off): 2 mm discs onto 1 ml pipettes, 3 mm discs onto 2 ml pipettes, and 5 mm discs onto 5 ml pipettes. This was accomplished by removing the cotton plug inside the pipette, passing a piece of thread through the pipette, looping it through a biofelt disc on the end, and then passing the thread hack through the pipette in the reverse direction. The cotton plug was then replaced so that the biofelt disc could be held in position on the end of the pipette.

ERL fibrinogen was formulated in CFB and adjusted to a final fibrinogen concentration of 37.5 mg/ml with a pH of 7.4±0.1. Thrombin (manufactured in-house) was formulated in CTB and adjusted to a final thrombin concentration of 0.1 units/mg of fibrinogen or 25 Units/ml thrombin, with a final pH of 7.4±0.1. Once prepared, the final fibrinogen and thrombin solutions were placed on ice and cooled to 4° C.±2° C.

All of the pipette applicators were manufactured by adding 0.133 ml of fibrinogen (at 4° C.±2° C.) and 0.02 ml of thrombin (at 4° C.±2° C.) to a 5 ml round-bottom polypropylene tube (12 mm×75 mm). The contents of each tube were mixed by hand and the biofelt-covered disc on the end of the pipette was inserted into the tube and allowed to absorb the fibrinogen and thrombin mixture for 20 seconds. The pipette was then removed and transferred into a clean 5 ml round-bottom tube which was immediately immersed in liquid nitrogen and frozen for 1 minute. The frozen applicators with the FAST hemostatic material were then placed at −80° C. until lyophilization.

Additional cotton-tipped wooden applicators were also produced. To manufacture these applicators, 0.192 ml of fibrinogen (at 4° C.±2° C.) and 0.02 ml of thrombin (at 4° C.±2° C.) were added to a 1.5 ml microcentrifuge tube. The contents of each tube were mixed by hand and the cotton-tipped end of a long wooden applicator was inserted into the tube and allowed to absorb the mixture for 20 seconds. The microcentrifuge tube containing the applicator was then immersed in liquid nitrogen and frozen for 1 minute. The frozen applicators were placed at −80° C. until lyophilization.

The same fibrinogen and thrombin solutions used to manufacture the hemostatic material with applicators were also used to manufacture FDs (1.5 cm×1.5 cm) with DEXON™ as a backing material. These FDs were tested in the EVPA and Adherence assays and all passed 100%, with adherence scores of 4.0.

Two of the 1 ml pipettes (with the 2 mm biofelt discs attached) were then tested for effectiveness in vivo. For each assessment a small piece of tissue was cut from the liver of a pig and the applicator was pressed firmly against the injury site. It was held in place for 2 minutes and then the thread was released so that the biofelt disc could remain on the injury site while the pipette was pulled away. In both of these tests, the biofelt discs adhered to the injury site and hemostasis was achieved.

Hemostatic test materials were manufactured using the thick type of (250 mg/cc) PGA biofelt material from Concordia Medical. The biofelt was cut into 2, 3, or 5 mm diameter discs which were then attached to the ends of serological pipettes (with the tapered ends cut off): 2 mm discs onto 1 ml pipettes, 3 mm discs onto 2 ml pipettes, and 5 mm discs onto 5 ml pipettes. This was accomplished by removing the cotton plug inside the pipette, passing a piece of thread through the pipette, looping it through a biofelt disc on the end, and then passing the thread back through the pipette in the reverse direction. The cotton plug was then replaced so that the biofelt disc could be held in position on the end of the pipette.

Two different formulations of fibrinogen were prepared. First, fibrinogen (front CSL Behring) was formulated in CSLFB and adjusted to a final fibrinogen concentration of 37.5 mg/ml with a pH of 7.4±0.1. Second, fibrinogen (from CSL Behring) was formulated in CSLFB and then underwent glycine precipitation according to the procedure in Okuda et al: A New Method of Purifying Fibrinogen with Both Biological and Immunological Activity from Human Plasma. Preparative Biochemistry & Biotechnology; 2003; 33(4);239-252. The precipitated fibrinogen was then resuspended in CFB and adjusted to a final fibrinogen concentration of 37.5 mg/ml with a pH of 7.4±0.1. Thrombin (manufactured in-house) was formulated in CTB and adjusted to a final thrombin concentration of 0.25 units/mg of fibrinogen or 62.5 Units/ml, with a final pH of 7.4−0.1. Once prepared, the final fibrinogen and thrombin solutions were placed on ice and cooled to 4° C.±2° C.

All of the pipette applicators were manufactured by adding 0.270 ml of fibrinogen (at 4° C.±2° C.) and 0.043 ml of thrombin (at 4° C.±2° C.) to a 5 ml round-bottom polypropylene tube (12 mm×75 mm). However, half of the applicators were made using the standard CSL fibrinogen while the others were made using the glycine-precipitated CSL fibrinogen. The contents of each tube were mixed by hand and the biofelt-covered disc on the end of the pipette applicator was inserted into the tube and allowed to absorb the fibrinogen and thrombin mixture for 25 seconds. The tube containing the applicator was then immediately immersed in liquid nitrogen and frozen for 1 minute. The frozen applicators were placed at −80° C. until lyophilization.

Additional cotton-tipped wooden applicators were also produced. Once again, half of the applicators were made using the standard CSL fibrinogen the others were made using the glycine-precipitated CSL fibrinogen. To manufacture these applicators, 0.270 ml of fibrinogen (at 4° C.±2° C.) and 0.043 ml of thrombin (at 4° C.±2° C.) were added to a 1.5 ml microcentrifuge tube. The contents of each tube were mixed by hand and the cotton-tipped end of a long wooden applicator was inserted into the tube and allowed to absorb the mixture for 20 seconds. The microcentrifuge tube containing the applicator was then immersed in liquid nitrogen and frozen for 1 minute. The frozen applicators were placed at −80° C. until lyophilization.

The same fibrinogen and thrombin solutions used to manufacture the kittners and applicators were also used to manufacture FDs (100 cm×100 cm) with DEXON™ as a backing material. These FDs were tested in the EVPA and Adherence assays and all passed 100%, with adherence scores of 4.0.)

### Example 16

Multiple types of applicators were manufactured by attaching different materials to the ends of 1 ml and/or 2 ml serological pipettes (with the tapered ends cut off). The materials used included DEXON™, calcium alginate, Superstat® modified collagen, and PGA BIOFELT®, which were all cut into discs, as well as Gelfoam® and a puffed cornstarch material which were cut into thicker plug shapes. These materials were all attached to the pipette ends by looping a piece of thread through the material on the end of the pipette, and then passing the thread ends back through the pipette. The cotton plug was then inserted to hold the material on the end of the pipette. Additionally, circular pieces of the plastic hook surface of Velcro were cut and glued onto the ends of 1 ml and 2 ml serological pipettes. PGA BIOFELT® discs were then pressed onto several of these Velcro ends. Cotton-tipped wooden applicators were also used.

ERL fibrinogen was formulated in CFB and adjusted to a final fibrinogen concentration of 37.5 mg/ml with a pH of 7.4±0.1. A yellow dye was then added to the fibrinogen solution. Recombinant thrombin (RECOTHROM®) was reconstituted with the supplied diluent (0.9% sodium chloride) according to the manufacturer's instructions to a concentration of 1000 units/ml with a pH of 6.0±0.1. A portion of this thrombin solution was also dilated in CTB and adjusted to a final thrombin concentration of 0.1 units/mg of fibrinogen or 25 units/ml thrombin, with a final pH of 7.4±0.1. A blue dye was added to both thrombin solutions. Once prepared, the final fibrinogen and thrombin solutions were placed on ice and cooled to 4° C.±2° C.

An applicator of each type was then prepared under each of the following conditions: mixed thrombin and fibrinogen, thrombin alone, and fibrinogen alone. For the mixed thrombin and fibrinogen group, 0.043 ml of the 25 units/ml thrombin solution (at 4° C.±2° C.) was added to a 5 ml round-bottom polypropylene tube (12 mm×75 mm), followed by 0.27 ml of the fibrinogen solution (at 4° C.±2° C.). For the cotton-tipped wooden applicators, the thrombin and fibrinogen solutions were added to a 1.5 ml microcentrifuge tube instead of the 5 ml round-bottom tube. The tubes were then briefly tapped to fully mix the two solutions, which appeared green upon mixing. The tip of an applicator was inserted into each tube and allowed to absorb the thrombin and fibrinogen mixture for 10 seconds. The tubes were then immediately immersed in liquid nitrogen and frozen tor 30 seconds.

The thrombin alone and fibrinogen alone groups were manufactured in a similar manner to the mixed thrombin and fibrinogen group. For the thrombin alone condition, 0.313 ml of the 1000 units/ml thrombin solution (at 4° C.±2° C.) was added to a 5 ml round-bottom polypropylene tube (12 mm×75 mm) or a 1.5 ml microcentrifuge tube. For the fibrinogen alone condition, 0.27 ml of the fibrinogen solution (at 4° C.±2° C.) was added to each tube. The tip of an applicator was then inserted into each tube and allowed to absorb the thrombin or fibrinogen solutions for 10 seconds. The tubes were then immediately immersed in liquid nitrogen and frozen for 30 seconds. After freezing, the applicators were all placed at −80° C. for at least two hours before being lyophilized in the freeze-dryer.

The different applicators with the hemostatic test materials were then evaluated for effectiveness in vivo. For each assessment a small piece of tissue was removed from the spleen of a pig using either a biopsy punch or scissors, and the applicator was pressed firmly against the injury site and held in place for 5 minutes. The thread was released so that the biofelt disc could remain on the injury site while the pipette was pulled away for the 2 mL pipette applicators with biofelt pads. The results of this in vivo experiment are presented below in Table 1. An example of the in vivo evaluation using the 2 mL pipette with a biofelt pad is presented below in FIG. 1.

TABLE 1
Summary Data for Mild Splenic Injury: Various Applicator Devices (9 May 2011)

| | | Injury Size, | Bleeding | Bleeding | | | |
|---|---|---|---|---|---|---|---|
| Animal # | Injury # | Method | Severity | Description | Applicator Device | Actives Formulation | Hemostasis? |
| 2 | 2 | 2.0 mm Biopsy | Mild | Oozing | 2 mL Pipette | FAST with Zymo | No |
| | | Punch | | | String/STB Backer | Thrombin | |
| | | | | | Panel | | |
| 2 | 3 | 2.0 mm Biopsy | Mild | Oozing | 2 mL Pipette | Zymo Thrombin | No |
| | | Punch | | | String/STB Backer | | |
| | | | | | Panel | | |
| 2 | 4 | 2.0 mm Biopsy | Mild | Oozing | 2 mL Pipette | Zymo Thrombin | Yes |
| | | Punch | | | String/Puffed | | |
| | | | | | Cornstarch | | |
| 2 | 5 | 2.0 mm Biopsy | Mild | Oozing | 2 mL Pipette | FAST with Zymo | Yes |
| | | Punch | | | String/GelFoam | Thrombin | |
| 2 | 6 | 2.0 mm Biopsy | Mild | Oozing | 2 mL Pipette | Zymo Thrombin | Partial, slow ooze |
| | | Punch | | | String/GelFoam | | |
| 2 | 7 | 2.0 mm Biopsy | Moderate | Flowing | 2 mL Pipette | ERL Fibrinogen | Partial, slow ooze |
| | | Punch | | | String/GelFoam | | |
| 2 | 8 | 2.0 mm Biopsy | Mild | Oozing | 2 mL Pipette | FAST with Zymo | No |
| | | Punch | | | String/SuperStat | Thrombin | |
| 2 | 9 | 2.0 mm Biopsy | Moderate | Flowing | 2 mL Pipette | FAST with Zymo | No |
| | | Punch | | | String/Calcium | Thrombin | |
| | | | | | Alginate | | |
| 2 | 10 | 2.0 mm Biopsy | Mild | Oozing | 2 mL Pipette | FAST with Zymo | No |
| | | Punch | | | Velcro Hooks | Thrombin | |
| 2 | 12 | 2.0 mm Biopsy | Mild | Oozing | Cotton-tipped | FAST with Zymo | No |
| | | Punch | | | Wooden Applicator | Thrombin | |
| 1 | 5 | 5 mm Scissors | Mild | Oozing | 2 mL Pipette | FAST with Zymo | Yes, with slight |
| | | | | | String/Biofelt | Thrombin | leak |
| 1 | 6 | 2.0 mm Biopsy | Mild | Oozing | 2 mL Pipette | FAST with STB | Yes |
| | | Punch | | | String/Biofelt | Thrombin | |
| 1 | 7 | 2.0 mm Biopsy | Mild | Oozing | 2 mL Pipette | Zymo Thrombin | Yes |
| | | Punch | | | String/Biofelt | | |
| 2 | 1 | 2.0 mm Biopsy | Mild | Oozing | 2 mL Pipette | ERL Fibrinogen | No |
| | | Punch | | | String/Biofelt | | |
| 2 | 11 | 2.0 mm Biopsy | Mild | Oozing | 1 mL Pipette | FAST with Zymo | No |
| | | Punch | | | String/Biofelt | Thrombin | |
| 2 | 15 | 2.0 mm Biopsy | Mild | Oozing | 2 mL Pipette | FAST with STB | Yes |
| | | Punch | | | String/Biofelt | Thrombin | |
| 2 | 16 | 2.0 mm Biopsy | Mild | Oozing | 2 mL Pipette | FAST with STB | Yes |
| | | Punch | | | String/Biofelt | Thrombin | |

### Example 17

ERL fibrinogen was formulated in CFB and adjusted to a final fibrinogen concentration of 37.5 mg/ml with a pH of 7.4±0.1. Thrombin (manufactured in-house) was formulated in CTB and adjusted to a final thrombin concentration of 0.1 units/mg of fibrinogen or 25 Units/ml thrombin, with a final pH of 7.4−0.1. Once prepared, the final fibrinogen and thrombin solutions were placed on ice and cooled to 4° C.±2° C.

Cylindrical molds were prepared by cutting off the luer-lock ends of 3, 10, and 20 ml polypropylene syringes (Becton Dickinson) and withdrawing the syringe plungers to the 2.5, 8, and 15 ml markings, respectively. The molds were then placed upright and surrounded by ice, leaving the open ends exposed at the top, and resorbable DEXON™ backing material was added to the molds as a support material. For half of these molds, the DEXON™ backing material was shredded into small pieces of approximately 1 mm×1 mm in size which were placed into the syringe; for the rest the DEXON™ backing material was kept intact and rolled into a tube which was slid down into the syringe barrel.

The 3 ml syringes were manufactured by dispensing 0.20 ml of thrombin (at 4° C.±2° C.) followed by 13 ml of fibrinogen (at 4° C.±2° C.) into the cooled syringes. The 10 ml and 20 ml syringes were made in the same manner but using 0.82 ml of thrombin with 6.0 ml of fibrinogen for the 10 ml syringes and 1.64 ml of thrombin with 12.0 ml of fibrinogen for the 20 ml syringes. Immediately alter each syringe was filled, it was removed from the ice and the contents were mixed by placing a thumb over the opening and inverting the syringe 3 times. The syringe was then immersed in liquid nitrogen and frozen for 2 minutes. After freezing, the syringes were placed at −80° C. for at least two hours before being lyophilized in the freeze-dryer.

Additionally, 12 mm×75 mm and 17 mm×100 mm polypropylene tubes were also used as molds. These tubes were placed on ice to cool. For the 12 mm×75 mm tubes, 0.41 ml of thrombin (at 4° C.±2° C.) and 2.59 ml of fibrinogen (at 4° C.±2° C.) were dispensed while for the 17 mm×100 mm tubes, 0.68 ml of thrombin (at 4° C.±2° C.) and 4.32 ml of fibrinogen (at 4° C.±2° C.) were dispensed into the cooled tubes. Immediately after each tube was filled, it was removed from the ice and the contents were mixed by placing a thumb over the opening and inverting the tube 3 times. The tube was then immersed in liquid nitrogen and frozen for 2 minutes. After freezing, the syringes were placed at −80° C. for at least two hours before being lyophilized in the freeze-dryer.

Pigs were anesthetized and rendered cold and coagulopathic according to the method of: Bochicchio G, Kilbourne M, Kuehn R, Keledjian K, Hess J, Scalea T. Use of a modified chitosan dressing in a hypothermic coagulopathic grade V liver injury model. Am J Surg 2009; I98:617e22. The pigs were then given a Grade V thru and thru liver injury by use of a 1″ diameter electric drill into the fundus of the liver. The resulting injury was a full thickness wound that included laceration of multiple large blood vessels with a significant blood loss and corresponding drop in blood pressure.

Immediately following injury, the injury site was treated with a 20 ml syringe described above. The first animal was treated with a syringe filled with material that included shredded backing material. The second was treated with material containing an intact rolled-up sheet of backing material.

The material was applied by inserting the open end of the syringe into the liver wound, and advancing the plunger of the syringe to expel the rod-like material within, while simultaneously withdrawing the syringe barrel from the wound site in order to deliver the material to the entire depth of the wounded tissue. Once this application was complete the manual pressure was applied to the wound site for approximately 150 seconds.

Upon removal of pressure complete hemostasis was observed. The animals were observed for approximately one hour, during which hemostasis was uninterrupted and their blood pressures stable.

### Example 18

ERL fibrinogen was formulated in CFB and adjusted to a final fibrinogen concentration of 37.5 mg/ml with a pH of 7.4±0.1. Thrombin (manufactured in-house): was formulated in CTB and adjusted to a final thrombin concentration of 0.1 units/mg of fibrinogen or 25 Units/ml thrombin, with a final pH of 7.4±0.1. Once prepared, the final fibrinogen and thrombin solutions were placed on ice and cooled to 4° C.±2° C.

Cylindrical molds were prepared by cutting off the luer-lock ends of 1, 3, 10, 20, 30, and 60 ml polypropylene syringes (Becton Dickinson) and withdrawing the syringe plungers to their respective full volume capacities. Additional molds were made using cylindrical open top molds with screwed-advance plungers. The molds were then placed upright and surrounded by ice, leaving the open ends exposed at the top, and resorbable DEXON™ backing material was added to the molds as a support material. For these molds, the DEXON™ backing material was cut into discs corresponding to the approximate diameters of each syringe size. Each disc were then pushed down into the syringe barrel and positioned on top of the plunger.

The syringes were manufactured by mixing the thrombin (at 4° C.±2° C.) and fibrinogen (at 4° C.±2° C.) solutions in the volumes shown below in Table 1 and then transferring the mixtures to the cooled syringes. Immediately after each syringe was filled, it was immersed in liquid nitrogen and frozen for 2 minutes. After freezing, the syringes were placed at −80° C. for at least two hours before being lyophilized in the freeze-dryer.

TABLE 1
Dimensions and Volumes of Syringe Molds

| Syringe | | | | | Fibrinogen | Thrombin | Total | Total | Total |
|---|---|---|---|---|---|---|---|---|---|
| Size | Dia. | Radius | Length | Vol. | Vol. | Vol. | Vol. | Fibrinogen | Thrombin |
| (ml) | (mm) | (mm) | (mm) | (mm³) | (ml) | (ml) | (ml) | (g) | (units) |
| 1 | 4.78 | 2.39 | 50 | 897.2 | 0.80 | 0.12 | 0.92 | 0.03 | 2.99 |
| 3 | 8.66 | 4.33 | 50 | 2945 | e2.65 | 0.40 | 3.05 | 0.10 | 9.95 |
| 10 | 14.5 | 7.25 | 60 | 9907.8 | 8.75 | 1.31 | 10.0 | 0.33 | 32.83 |
| 20 | 19.1 | 9.565 | 70 | 20119.5 | 17.50 | 2.63 | 20.1 | 0.66 | 65.67 |
| 30 | 21.7 | 10.85 | 80 | 29586.9 | 26.25 | 3.94 | 30.1 | 0.98 | 98.50 |
| 60 | 26.7 | 13.35 | 110 | 61589.3 | 52.50 | 7.88 | 60.3 | 1.97 | 197.00 |

Pigs were anesthetized and rendered cold and coagulopathic as described in the previous Example. Grade 3 injuries to the liver were created using surgical scissors. In the first treatment a 1 ml syringe was used and the fibrin sealant “putty” extruded into the injury site and pressed into place by fingertips of a hand wearing surgical gloves. After approximately 2 minutes of manual pressure the hand was removed. The fibrin in the wound did not stick or adhere to the surgical gloves, and hemostasis was achieved where the material had been applied. This process was repeated at another site using the fibrin sealant “putty” extruded by the screw-type plunger applicator. Again manual pressure for approximately 2 minutes was applied using a gloved hand directly on the putty. The result was hemostasis, and the putty did not adhere to the glove. Similar results were also obtained from injury site son the spleen and kidney, using putty from several different applicators.

Success from these trials constitutes justification to further optimize their properties and production processes. Optimization of the Thrombin:Fibrinogen ratio, fibrinogen dose, freezing media (ie liquid nitrogen, dry ice/alcohol bath, liquid nitrogen vapour etc), mixing processes such as pre-filling the syringe with one of the two solutions, dispensing of pre-mixed or individual solutions, the effects of various mold sizes and geometries, optimal residual moisture levels and the calibrated return of moisture into the dried product and freeze-drying cycle will be investigated.

ERL fibrinogen was formulated in CFB and adjusted to a final fibrinogen concentration of 37.5 mg/ml with a pH of 7.4±0.1. Thrombin (manufactured in-house) was formulated in CTB and adjusted to three thrombin concentrations: 1.0. units/mg of fibrinogen (or 250 Units/ml thrombin), 0.1 units/mg of fibrinogen (or 25 Units/ml thrombin), and 0.01 units/mg of fibrinogen (or 2.5 Units/ml thrombin), all of which were adjusted to a final pH of 7.4±0.1. Once prepared, the final fibrinogen and thrombin solutions were placed on ice and cooled to 4° C.±2° C.

Cylindrical molds were prepared by cutting off the luer-lock ends of 1, 3, and 10 ml polypropylene syringes (Becton Dickinson) and withdrawing the syringe plungers to their respective full volume capacities. The syringes were then placed upright and surrounded by ice, leaving the open ends exposed at the top. The 1 ml syringes were manufactured by mixing 0.14 ml of thrombin (at 4° C.±2° C.) and 0.86 ml of fibrinogen (at 4° C.±2° C.) and transferring the mixture to the cooled syringes. The 3 ml and 10 ml syringes were made in the same manner but using 0.41 ml of thrombin with 2.59 ml of fibrinogen for the 3 ml syringes and 1.36 ml of thrombin with 8.64 ml of fibrinogen for the 10 ml syringes. Immediately after each syringe was filled, it was immersed in liquid nitrogen and frozen for 2 minutes. After freezing, the syringes were placed in a −80° C. freezer for at least two hours before being lyophilized in the freeze-dryer.

A splenic injury was created fey using a hemostat to pierce the spleen and create an opening down into the organ, and then expanding it using the hemostat in order to produce mild to moderate bleeding, avoiding pulsatile bleeding if possible. Initial bleeding was assessed as mild to moderate or pulsatile for approximately 30 seconds. Shed blood was suctioned from the cavity, and a 3 mL syringe was applied with manual pressure to the injured surface of the spleen for 3 minutes, followed by compression and examination to record the effects of treatment. The initial treatment was determined by the surgeon to include at least 1 syringe inside the wound, followed by 3 minutes of manual compression while holding the spleen together. Hemostasis was evaluated immediately after the cessation of application pressure. The results of this evaluation are presented below in Table 1.

### Example 19

TABLE 1
Summary Data for the Mild to Moderate Splenic Injury

| | Syringe | Thr:Fib | Hemostasis | Hemostasis | | |
|---|---|---|---|---|---|---|
| | Size | (IU Thr/mg | After 1^{st} | After 2^{nd} | Total # | |
| Animal # | (mL) | Fib) | Application? | Application? | Used | Comments |
| 4 | 3 | 0.01 | Yes | — | 2 | Slow to set |
| | | | | | | up |
| 4 | 3 | 0.1 | Yes | — | 2 | |
| 1 | 3 | 1.0 | No | No | 2 | Set up too |
| | | | | | | fast |
| | | | | | | Stuck to |
| | | | | | | gauze |

The liver injury used to test 3 mL syringes was performed in a manner similar to the splenic injury model above. The liver of each subject was injured using a hemostat to pierce the liver and create an opening down into the organ. Treatment consisted of the application of a 3 mL syringe to the injury site of the liver, followed by 3 minutes of manual compression while holding the liver together and examination to record the effects of treatment. Hemostasis was evaluated immediately after the cessation of application pressure. The results of this evaluation are presented below in Table 2.

Another liver injury was also performed and was created using a drill with a 1″ auger bit, with the goal of producing a Grade 3 or greater hepatic injury. Treatment consisted of the application of a 10 mL syringe to the injured surfaces of the liver, followed by compression and examination to record the effects of treatment. The initial treatment was determined by the surgeon to include at least 1 syringe inside the wound, followed by 3 minutes of manual compression while holding the liver together.

Hemostasis was evaluated immediately after the cessation of application pressure, and 5 minutes after the initial application of the syringe. The results of this evaluation are also presented below in Table 2.

TABLE 2
Summary Data for the Liver Injury Model

| | Syringe | Thr:Fib | Hemostasis | Hemostasis | | |
|---|---|---|---|---|---|---|
| | Size | (IU Thr/mg | After 1^{st} | After 2^{nd} | Total # | |
| Animal # | (mL) | Fib) | Application? | Application? | Used | Comments |
| 7 | 3 | 0.1 | Yes | — | | |
| 7 | 3 | 0.1 | Yes | — | | |
| 7 | 10 | 0.1 | Yes | — | | Used syringe |
| | | | | | | and FD |
| 7 | 10 | 0.1 | Yes | — | | Used syringe |
| | | | | | | and FD |

The kidney injury used to test 3 mL syringes was performed in a manner similar to the splenic injury model above. The kidney of each subject was injured using a hemostat to pierce the liver and create an opening down into the organ.

Treatment consisted of the application of a 3 mL syringe to the injury site of the kidney, followed by 3 minutes of manual compression while holding the kidney together and examination to record the of feels of treatment. Hemostasis was evaluated immediately after the cessation of application pressure. The results of this evaluation are presented below in Table 3.

TABLE 3
Summary Data for the Kidney Injury Model

| | Syringe | Thr:Fib | Hemostasis | Hemostasis | | |
|---|---|---|---|---|---|---|
| | Size | (IU Thr/mg | After 1^{st} | After 2^{nd} | Total # | |
| Animal # | (mL) | Fib) | Application? | Application? | Used | Comments |
| 8 | 3 | 0.01 | Yes | — | | Slow to set |
| | | | | | | up |
| 8 | 3 | 0.01 | Yes | — | | Slow to set |
| | | | | | | up |

ERL fibrinogen was formulated in CFB and adjusted to a final fibrinogen concentration of 37.5 mg/ml with a pH of 7.4±0.1. The fibrinogen was then formulated with one of each of the following additives: 1,2-Propanediol/Propylene Glycol, Glycerol, PEG 400, Poly Propylene Glycol 425, Poly Propylene Glycol 3500, Sorbitol, Dibutyl Sebacate, DL-Alpha-Monoolein, Dextrin, D-Mannitol, Trehalose, or PEG 4000. Thrombin (manufactured in-house) was formulated in CTB and adjusted to a thrombin concentration of 0.1 units/mg of fibrinogen (or 25 Units/ml thrombin) and a final pH of 7.4±0.1. Once prepared, the final fibrinogen and thrombin solutions were placed on ice and cooled to 4° C.±2° C.

Cylindrical molds were prepared by cutting off the leur-lock ends 3 ml polypropylene syringes (Becton Dickinson) and withdrawing the syringe plungers to their full volume capacities. The syringes were then placed upright and surrounded by ice, leaving the open ends exposed at the top. The 3 ml syringes were manufactured by mixing 0.41 ml of thrombin (at 4° C.±2° C.) and 2.59 ml of fibrinogen (at 4° C.±2° C.) and transferring the mixture to the cooled syringes. Immediately after filling each syringe was frozen, with half of the syringes from each condition being frozen by immersion in liquid nitrogen and the other half by immersion in a dry ice/methanol mixture. After freezing, the syringes were placed at −80° C. for at least two hours before being lyophilized in the freeze-dryer.

The liver injuries were achieved with the use of scissors or a knife and were made deep enough to produce mild to moderate bleeding, avoiding pulsatile bleeding if possible. Initial bleeding was assessed as mild to moderate or pulsatile for approximately 30 seconds. Shed blood was suctioned from the peritoneal cavity, and the syringe was applied with manual pressure to the injured surface of the spleen for 3 minutes. Hemostasis was evaluated immediately after the cessation of application pressure, and 5 minutes after the initial application of the syringe. The results of this evaluation are presented below in Table 1.

TABLE 1
Summary Data for Mild to Moderate Liver Injury: Syringe Putty-type Devices,
9 May 2011

| | | Injury Size, | Bleeding | Bleeding | Actives | | |
|---|---|---|---|---|---|---|---|
| Animal # | Injury # | Method | Severity | Description | Formulation | Additive | Hemostasis? |
| 1 | 17 | 2.2 × 0.5 mm | Mild to | Oozing, | FAST with | Dibutyl | Yes |
| | | Scissors | Moderate | Flowing | Zymo | Sebacate | |
| | | | | | Thrombin | | |
| 1 | 18 | 2 × 1 mm | Mild to | Oozing, | FAST with | Dibutyl | Yes |
| | | Scissors | Moderate | Flowing | Zymo | Sebacate | |
| | | | | | Thrombin | | |
| 1 | 19 | 2 × 1 × 3 mm | Mild to | Oozing, | FAST with | Dibutyl | No |
| | | Scissors | Moderate | Flowing | Zymo | Sebacate | |
| | | | | | Thrombin | | |
| 1 | 20 | Scissors | Mild to | Oozing, | FAST with | Propylene | No |
| | | | Moderate | Flowing | Zymo | Glycol | |
| | | | | | Thrombin | | |
| 1 | 21 | Scissors | Mild to | Oozing, | FAST with | Dextrin | Partial, |
| | | | Moderate | Flowing | Zymo | | slight |
| | | | | | Thrombin | | oozing |
| 1 | 22 | 2 × 1 × 4 mm | Mild to | Oozing, | FAST with | Glycerol | No |
| | | Scissors | Moderate | Flowing | Zymo | | initially, |
| | | | | | Thrombin | | Yes later |
| 1 | 23 | Scissors | Mild to | Oozing, | FAST with | PEG 400 | No |
| | | | Moderate | Flowing | Zymo | | |
| | | | | | Thrombin | | |
| 1 | 24 | 1.5 mm | Mild to | Oozing, | FAST with | PEG 400 | No |
| | | Scissors | Moderate | Flowing | Zymo | | |
| | | | | | Thrombin | | |
| 1 | 25 | 1.5 × 0.5 × 1 mm | Mild to | Oozing, | FAST with | Polypropylene | Yes, |
| | | Scissors | Moderate | Flowing | Zymo | Glycol | oozing in |
| | | | | | Thrombin | 425 | gauze area |
| 2 | 17 | Scissors | Mild to | Oozing, | FAST with | Polypropylene | Yes, |
| | | | Moderate | Flowing | Zymo | Glycol | oozing in |
| | | | | | Thrombin | 3500 | gauze area |
| 2 | 18 | Scissors | Mild to | Oozing, | FAST with | Sorbitol | Yes, |
| | | | Moderate | Flowing | Zymo | | oozing in |
| | | | | | Thrombin | | gauze area |
| 2 | 19 | Scissors | Mild to | Oozing, | FAST with | Dibutyl | Yes, |
| | | | Moderate | Flowing | Zymo | Sebacate | oozing in |
| | | | | | Thrombin | | gauze area |
| 2 | 20 | Scissors | Mild to | Oozing, | FAST with | DL-a- | Yes, |
| | | | Moderate | Flowing | Zymo | Monoolein | oozing in |
| | | | | | Thrombin | | gauze area |
| 2 | 21 | Scissors | Mild to | Oozing, | FAST with | D-Mannitol | Yes, |
| | | | Moderate | Flowing | Zymo | | oozing in |
| | | | | | Thrombin | | gauze area |
| 2 | 22 | Scissors | Mild to | Oozing, | FAST with | Trehalose | Yes, |
| | | | Moderate | Flowing | STB Thrombin | | oozing in |
| | | | | | | | gauze area |
| 2 | 23 | Scissors | Mild to | Oozing, | FAST with | PEG 4000 | Yes, |
| | | | Moderate | Flowing | STB Thrombin | | oozing in |
| | | | | | | | gauze area |
| 2 | 24 | Scissors | Mild to | Oozing, | FAST with | PEG 400 | Yes, |
| | | | Moderate | Flowing | Zymo | | oozing in |
| | | | | | Thrombin | | gauze area |
| 2 | 25 | Scissors | Mild to | Oozing, | FAST with | None | Yes, |
| | | | Moderate | Flowing | Zymo | | oozing in |
| | | | | | Thrombin | | gauze area |
| 2 | 26 | Scissors | Mild to | Oozing, | FAST with | None | Yes, |
| | | | Moderate | Flowing | Zymo | | oozing in |
| | | | | | Thrombin | | gauze area |
| 2 | 27 | Scissors | Mild to | Oozing, | FAST with | Dextrin | Yes, |
| | | | Moderate | Flowing | Zymo | | oozing in |
| | | | | | Thrombin | | gauze area |
| 2 | 28 | Scissors | Mild to | Oozing, | FAST with | Dibutyl | Yes, |
| | | | Moderate | Flowing | Zymo | Sebacate | oozing in |
| | | | | | Thrombin | | gauze area |
| 2 | 29 | Scissors | Mild to | Oozing, | FAST with | Polypropylene | Yes, |
| | | | Moderate | Flowing | Zymo | 3500 | oozing in |
| | | | | | Thrombin | | gauze area |

### Example 21

In order to assess the performance of hemostatic applicators in vitro, the Mild to Moderate Hemorrhage Assay (MMHA) was developed to more closely model the application of a hemostatic agent to an injury site using an applicator compared to the previously developed EVPA assay. This assay could then be used to evaluate the ability of the hemostatic applicator to stop the flow of fluid through a hole(s) in an animal tissue or tissue-like substrate. The assay could be adapted for use with a variety of tissues or tissue-like substrates and could be used to model different types of bleeding by varying the size and number of holes made in the tissue substrate, as well as the flow rate of the fluid facing pumped through it.

The syringe, barrel is first filled with buffered saline solution warmed to 37° C. The surface of the tissue substrate, normally a piece of sausage casing soaked in buffered saline solution, is scraped and placed on top of the syringe barrel with the scraped side facing upwards. The Plexiglass panel is placed on top of the syringe and secured with screws and wingnuts so that the sausage casing is held tightly between the O-ring and the syringe barrel. A hole is then made in the middle of the casing and the peristaltic pump is turned on so that some of the saline solution is pumped through the hole. The applicator to be tested is then pressed firmly against the casing hole for 3-5 minutes. Subsequently, the applicator is pulled away, the syringe is pressurized, and the performance of the hemostatic test material is evaluated according to the amount of pressure held without leaking. Afterwards, the casing can be removed from the apparatus and if a backing material is present, the adherence can be tested using a modified Adherence assay.

In order to test the performance of applicators in this assay, a pre-made FD was cut into small pieces of about 5 mm in diameter. The DEXON™ backing material was removed from some of the pieces while the rest had the backing material intact. These pieces were applied to a 2.8 mm hole in the sausage casing using one of the following applicators: a 2 ml serological pipette with a circular piece of the plastic hook surface of Velcro cut and glued onto the end, a 5 ml serological pipette with a flat-tipped silicone plug in the end, and a 5 ml serological pipette with a round-tipped silicone plug in the end. All of the applicators were held firmly against the casing holes for 5 minutes.

The results of this evaluation showed that when used with FD pieces that contained the backing material, all three applicator types held a pressure of 3 psi (˜150 mmHg). Additionally, the flat-tipped silicone and the Velcro applicators held for 3 minutes at 3 psi (˜150 mmHg) and 5 minutes at 5 psi (˜250 mmHg). The adherence was also tested and shown to be excellent, yielding adherence scores of 4.0.

The same FDs used for these applicators were also tested in the EVPA and Adherence assays. They all exhibited excellent performance, passing 100%, with adherence scares of 4.0.

### Example 22

Two of the thick BIOFELT® disc applicators described in Example 2 were tested for in vitro performance in MMHA. One was applied as manufactured, with the 2 ml serological pipette applicator shaft. Additionally, one of the BIOFELT® disc applicator tips was applied using a different applicator shaft: a 5 ml serological pipette with a flat-tipped silicone plug in the end. These applicators were applied to a 2.8 mm hole in MMHA and were held firmly against the hole for 5 minutes. Both applicators failed to hold pressure in this assessment; however, the adherence scores were 4.0, demonstrating that the applicators did adhere well to a tissue substrate in vitro.

Given that these applicators produced hemostasis when tested on mild to moderate injuries in vivo, the 2.8 mm hole used in this in vivo assay simulates a large injury and may have been too severe of a challenge for this type of applicator. Particularly since the manufacture of these applicators included transferring them into new tubes after a brief immersion in the fibrinogen/thrombin mixture, the BIOFELT® discs on the applicator ends may not have retained enough of the hemostatic material to contend with this bigger challenge.

Another style of applicator was also manufactured and tested for in vitro performance in the MMHA. An active component tip of about 5 mm in diameter was fashioned from a pre-made FD (from STB Lot#012011) and the PGA hacking material present on the FD piece was left intact. The active component tip was then used with an applicator shaft consisting of a 5 ml serological pipette with a flat-tipped silicone plug in the end. This applicator was applied to a 2.8 mm hole in MMHA and was held firmly against the hole for 5 minutes. The applicator tested in this assay held a range of pressures without leaking: 3 minutes at 2 psi (˜100 mmHg), 3 minutes at 3 psi (˜150 mmHg), and 3 minutes at 5 psi (˜250 mmHg). The adherence was also tested in a modified Adherence assay and shown to be excellent, generating an adherence score of 4.0.

FDs from the same lot (STB Lot#012011) used to make the applicator tested above, were also tested for performance in the EVPA and Adherence assays, as well as analyzed via gel electrophoresis. These FDs demonstrated excellent in vitro performance in the EVPA and Adherence assays, passing both assays 100% and yielding adherence scores of 4.0. In the clotting time gel electrophoresis analysis, pieces of an FD from the same lot were hydrated and allowed to clot for a certain period of time, at which point the reactions were quenched with a reducing solution. The reaction times varied from 15 seconds to 10 minutes, and included 13 time points in between. Any clots that had been formed were allowed to dissolve without further reaction. The solutions were then analyzed by gel electrophoresis as presented in FIGS. 1-2. Densitometry was used to quantify the fibrinogen chain conversions that had occurred at each time point. The time course data from this analysts was then compiled and a clotting profile was generated and is shown in FIG. 3. The profile illustrates that both Aα chain conversion and γ-γ dimer formation neared completion within 3 minutes, indicating rapid formation of a fibrin clot. The Bβ chain conversion progressed at a steady but slower rate of reaction.

FDs from Lot#012011, manufactured at fibrinogen doses of 11 and 13 mg/cm², were also assessed for performance in vivo. In this study, FDs were evaluated for their ability to achieve hemostasis in porcine models of both moderate liver and severe aortotomy injuries. For the moderate liver injury, a 4 cm diameter portion of the liver was excised, deep enough to produce moderate bleeding while avoiding pulsatile bleeding. Initial bleeding was assessed for approximately 30 seconds and shed blood was suctioned from the peritoneal cavity. A 2″×4″ FD containing 11 mg/cm² fibrinogen (from STB Lot#012011) was then applied with manual pressure to the injured surface of the liver for 5 minutes, at which point the degree of hemostasis was recorded.

For the aortotomy injury, the aorta was dissected free from the surrounding tissue and blood flow through the artery was occluded by tying off the artery above and below the injury site. A 4 mm aortic punch was then used to create a hole in the aorta. The ties were released and free pulsatile bleeding was allowed for 5 seconds. A 2″×4″ FD (from STB Lot#012011) was then applied to the injury site through the pool of blood while uncontrolled bleeding continued. The FD was pressed firmly against the wound for 5 minutes, at which point the degree of hemostasis was recorded. FDs at fibrinogen doses of both 11 and 13 mg/cm² were evaluated in this aortotomy injury model. For all of the FDs tested in both injury models, immediate and durable hemostasis was achieved.

### Example 23

Three varieties of Fabco® ENDOSTIK® endoscopic dissector sticks were used as pre-made applicators. These all consist of long plastic sticks with different styles of cotton tips. The ones used were the 5 mm kittner-dipped, 5 mm ballet-tipped, and the 10 mm cherry-tipped ENDOSTIKs®. In addition, two other types of applicators were manufactured. One type was made by cutting circular pieces of the fiber loop surface of Velcro® and super gluing them onto the ends of 2 ml serological pipettes (with the tapered ends cut off). These pieces were all cut to a diameter of approximately 6 mm so that they would match the diameter of the 2 ml pipettes. The second type was manufactured by cutting off 14 cm-long pieces of the plastic sticks from the ENDOSTIKs®. Circular pieces of the plastic book surface of Velcro® were next cut to a diameter of 5 mm and super glued onto the ends of the plastic sticks. PGA BIOFELT® discs (5 mm in diameter) were then pressed onto the Velcro® ends.

ERL fibrinogen was formulated in CFB and adjusted to a final fibrinogen concentration of 37.5 mg/ml with a pH of 7.4±0.1. A yellow dye was then added to the fibrinogen solution. Recombinant thrombin (Zymogenetic's RECOTHROM®) was reconstituted with the supplied diluent sodium chloride) according to the manufacturer's instructions to a concentration of 1000 units/ml with a pH of 6.0±0.1. A portion of this thrombin solution was also diluted in CTB and adjusted to a final thrombin concentration of 25 units/ml (for a ratio of 0.1 units thrombin/mg of fibrinogen), with a final pH of 2.4±0.1. A blue dye was added to both thrombin solutions. Once prepared, the final fibrinogen and thrombin solutions were placed on ice and cooled to 4° C.±2° C.

Applicators of each type were prepared according to each of the following conditions: mixed thrombin and fibrinogen, thrombin alone, and fibrinogen alone. For the mixed thrombin and fibrinogen groups, 0.043 ml of the 25 units/ml thrombin solution (at 4° C.±2° C.) and 0.27 ml of the fibrinogen solution (at 4° C.±2° C.) were added to either a 14 ml (17 mm×100 mm) round-bottom polypropylene tube for the cherry-tipped ENDOSTIKs® or a 5 ml (12 mm×75 mm) round-bottom polypropylene tube for all of the other applicators. The tubes were then briefly tapped to fully mix the two solutions, which appeared green upon mixing. The tip of an applicator was inserted into each tube and allowed to absorb the thrombin and fibrinogen mixture for 10 seconds. The tubes containing the applicators were then immediately frozen by immersion in a dry ice/alcohol bath for 3 minutes.

For the half of the applicators with the plastic hook Velcro® and BIOFELT® disc, after the fibrinogen and thrombin mixture was absorbed, the applicator was removed and transferred into a clean 5 ml round-bottom tube which was immediately frozen by immersion in a dry ice/alcohol bath for 3 minutes. After freezing, the applicators were placed at −80° C. for at least two hours before being lyophilized in the freeze-dryer. The thrombin alone and fibrinogen alone groups were manufactured in the same manner as the mixed thrombin and fibrinogen group except that only thrombin or fibrinogen was added to each tube. For the thrombin alone condition, 0.313 ml of the 1000 units/ml thrombin solution (at 4° C.±2° C.) was added to each tube and for the fibrinogen alone condition, 0.27 ml of the fibrinogen solution (at 4° C.±2° C.) was added to each tube.

The PGA BIOFELT® discs with the active components were securely held in place by the Velcro® hooks, but could be detached with a modest amount of force. The tip geometry of the applicators made using the round bottom tube was rounded, indicating that the active components on the tip can be molded into the desired shape.

The differed applicators with the hemostatic test materials were then evaluated for effectiveness in vivo. For each assessment a small piece of tissue was removed from the spleen of a pig using either a biopsy punch or scissors, and the applicator was pressed firmly against the injury site and held in place for 5 minutes. The results of this in vivo experiment are presented below in Table 1.

TABLE 1
Summary Data for Mild Splenic Injury: Endostick and 2 mL Pipette Applicator Devices (9 May 2011)

| | | Injury Size, | Bleeding | Bleeding | | | |
|---|---|---|---|---|---|---|---|
| Animal # | Injury # | Method | Severity | Description | Applicator Device | Actives Formulation | Hemostasis? |
| 1 | 1 | 1.5 mm Biopsy | Mild | Oozing | Flat Endostik | None | No |
| | | Punch | | | | | |
| 1 | 1 | 1.5 mm Biopsy | Mild | Oozing | Endostik, | FAST with Zymo | No |
| | | Punch | | | Velcro | Thrombin | |
| | | | | | Hooks/Biofelt | | |
| 1 | 2 | 2.0 mm Biopsy | Mild | Oozing | Endostik, | FAST with Zymo | No |
| | | Punch | | | Velcro | Thrombin | |
| | | | | | Hooks/Biofelt | | |
| 1 | 3 | 5 mm Scissors | Mild | Oozing | Endostik, | FAST with Zymo | No |
| | | | | | Velcro | Thrombin | |
| | | | | | Hooks/Biofelt | | |
| 1 | 4 | 5 mm Scissors | Mild | Oozing | Endostik, | FAST with Zymo | No |
| | | | | | Velcro | Thrombin | |
| | | | | | Hooks/Biofelt | | |
| 1 | 14 | 2.0 mm Biopsy | Mild | Oozing | 2 mL Pipette | FAST with Zymo | No |
| | | Punch | | | Velcro Soft Loops | Thrombin | |
| 1 | 8 | 2.0 mm Biopsy | Mild | Oozing | Cherry Endostick | None | No |
| | | Punch | | | | | |
| 1 | 9 | 2.0 mm Biopsy | Mild | Oozing | Cherry Endostick | ERL Fibrinogen | No |
| | | Punch | | | | | |
| 1 | 10 | 2.0 mm Biopsy | Mild | Oozing | Cherry Endostick | Zymo Thrombin | No |
| | | Punch | | | | | |
| 1 | 11 | 2.0 mm Biopsy | Mild | Oozing | Cherry Endostick | FAST with Zymo | No |
| | | Punch | | | | Thrombin | |
| 1 | 12 | 2.0 mm Biopsy | Mild | Oozing | Bullet Endostick | FAST with Zymo | No |
| | | Punch | | | | Thrombin | |
| 1 | 13 | 2.0 mm Biopsy | Mild | Oozing | Kittner Endostick | FAST with Zymo | No |
| | | Punch | | | | Thrombin | |

### Example 24

ERL fibrinogen was formulated in CFB and adjusted to a final fibrinogen concentration of 37.5 mg/ml with a pH of 7.4±0.1. Recombinant thrombin (Zymogenetic's RECOTHROM®) was reconstituted with the supplied diluent (0.9% sodium chloride) according to the manufacturers instructions to a concentration of 1000 units/ml with a pH of 6.0±0.1. The thrombin solution was then diluted in CTB and adjusted to a final thrombin concentration of 0.1 units/mg of fibrinogen (25 units/ml thrombin), with a final pH of 7.4±0.1. Once prepared, the 1 thrombin solutions were placed on ice and cooled to 4° C.±2° C.

Cylindrical molds were prepared by cutting off the luer-lock ends of 1, 3, 5, 20, 30, and 60 ml polypropylene syringes (Becton Dickinson) and withdrawing the syringe plungers to their respective full volume capacities. The syringes were then placed upright and surrounded by ice, leaving the open ends exposed at the top. The 1 ml syringes were manufactured by mixing 0.14 ml of thrombin (at 4° C.±2° C.) and 0.86 ml of fibrinogen (at 4° C.±2° C.) and transferring the mixture to the cooled syringes. The rest of the syringes were made in the same manner but using 0.41 ml of thrombin with 2.63 ml of fibrinogen for the 3 ml syringes, 0.69 ml of thrombin with 4.38 ml of fibrinogen for the 5 ml syringes, 2.75 ml of thrombin with 17.5 ml of fibrinogen for the 20 ml syringes, 4.13 ml of thrombin with 26.25 ml of fibrinogen for the 30 ml syringes, and 8.25 ml of thrombin with 52.50 ml of fibrinogen for the 60 ml syringes. Immediately after being filled, the 1, 3, 5, and 20 ml syringes were frozen by immersion in a dry ice/ethanol mixture for 5 minutes. The 30 and 60 ml syringes were frozen by immersion in liquid nitrogen for 1 minute. After freezing, the syringes were placed at −80° C. for at least two hours before being lyophilized in the freeze-dryer.

The splenic injuries were achieved with the use of scissors, while the liver injury was achieved with the use of a drill with at 1¼″ auger bit, with the goal of producing a Grade 3 or greater injury.

Treatment consisted of the application of a syringe appropriate to the size of the injured surface, followed by compression and examination to record the effects of treatment. The initial treatment was determined by the surgeon to include at least 1 syringe inside the wound, followed by 3 minutes of manual compression while holding the organ together.

Hemostasis was evaluated immediately after the cessation of application pressure, and 5 minutes after the initial application of the syringe. The results of this evaluation are presented below in Table 1.

TABLE 1
Summary Data for Moderate to Severe Splenic and Liver Injuries: Syringes (6 Jan. 2012

| | | | Injury | | | | | |
|---|---|---|---|---|---|---|---|---|
| | | | Size, | Bleeding | Bleeding | | Actives | |
| Animal # | Injury # | Organ | Method | Severity | Description | Applicator | Formulation | Hemostasis? |
| 1 | 1 | Spleen | 3 × 2 cm | Moderate | Flowing | 1 mL | FAST with | No (came |
| | | | Scissors | | | Syringe, | Zymo | off with |
| | | | | | | used 3 | Thrombin | Lap Pad) |
| 1 | 2 | Spleen | 3 × 2 cm | Moderate | Flowing | 3 mL | FAST with | Yes |
| | | | Scissors | | | Syringe | Zymo | |
| | | | | | | | Thrombin | |
| 1 | 3 | Spleen | 3 cm | Moderate | Flowing | 3 mL | FAST with | Yes |
| | | | Scissors | | | Syringe | Zymo | |
| | | | | | | | Thrombin | |
| 1 | 6 | Liver | 1¼ Auger | Severe | Pulsatile | 20 mL | FAST with | Yes |
| | | | Drill | | | Syringe, | Zymo | |
| | | | | | | used 3 | Thrombin | |
| 1 | 14 | Liver | 1¼ Auger | Severe | Pulsatile | 60 mL | FAST with | Yes |
| | | | Drill | | | Syringe, | Zymo | |
| | | | | | | used 2 | Thrombin | |

### Example 25

Hemostatic test materials (pellets) were made using a fibrinogen solution, mixed with either a gelatin solution or a starch solution. Gelatin was formulated to 10% in water and held at 37° C. Puffed cornstarch was dissolved in 20 ml of water to make a 20% starch solution and held at 37° C. ERL fibrinogen was formulated in CFB and adjusted to a final fibrinogen concentration of 37.5 mg/ml with a pH of 7.4±0.1.

For the 101% Gelatin condition, the gelatin and fibrinogen solutions were added to the wells of a 96 well flat-bottom plate as follows: rows 1 and 2; 150 ul of 10% gelatin followed by 150 ul of fibrinogen solution; row 3: 100 ul of 10% gelatin followed by 200 ul of fibrinogen solution; row 4: 25 ul of fibrinogen solution followed by 275 ul of 10% gelatin; row 5: 60 ul of 10% gelatin followed 240 ul of fibrinogen solution; row 6: 300 ul of fibrinogen solution alone; row 8: 240 ul of 10% gelatin followed 60 ul of fibrinogen solution. Row 7 was left empty. After the wells had been filled, the plate was frozen in a −80° C. freezer.

For the 20% Starch condition, a plate was prepared similar to the one for the Gelatin condition, except that the 20% Starch solution was substituted for the 10% Gelatin solution in all instances.

The plates were then lyophilized for 24 hours. Once the freeze-drying cycle was complete, the plates were removed and the pellets inside the wells were evaluated. Two types of evaluation were performed on the pellets. The first involved the ability of the pellet to be removed intact from the well. This was accomplished by inserting a small pipet tip into the center of the well and then attempting to lift out the pellet. The second evaluation was used to determine the wetting ability of the pellets. In this case, 0.075 ml of 37° C. saline solution was added to each pellet and the hydration of the pellet was observed.

Results:

Gelatin:

Rows 1, 3, 4 and 8 pellets were all hard to remove and very stiff. When liquid was applied, the pellets were all slow to wet. Row 5 pellets were easy to remove and wet easily. Row 6 was hard to remove, but wet very well.

Starch:

Pellets in all rows, with the exception of row 3, were fluffy and hard to remove from the wells, but wet well. Pellets in row 3 were easier to remove and wet.

Next, gelatin was formulated to 10% and 7.5% in water and held at 37° C. ERL fibrinogen was formulated in CFB and adjusted to a final fibrinogen concentration of 37.5 mg/ml with a pH of 7.4±0.1. The gelatin and fibrinogen solutions were added to the wells of a 96 well flat-bottom plate as follows: columns 2 through 12 received 100 ul of water, while the first four rows of column 1 received 200 ul of 10% gelatin solution and the last four rows of column 1 received 200 ul of 7.5% gelatin solution. Serial two-fold dilutions of the gelatin were performed, leaving 100 ul of gelatin in each well. Each well then received 100 ul of fibrinogen solution. The plate was then gently mixed, and placed on ice for 15 minutes. After the 15 minutes on ice, the temperature of the gelatin/fibrinogen mixture was 5° C. The plate was then moved to a −80° C. freezer. After 2 minutes in the −80° C. freezer, the temperature reached −1° C. Each well then received 15 ul of thrombin at 25 U/ml. The plate was then re-frozen and lyophilized.

Three types of evaluation were performed on the pellets. The first involved the ability of the pellets to be removed intact from the well. This was accomplished by insetting a small pipet tip into the center of the well and then attempting to lift out the pellet. The second evaluation was used to determine the wetting ability of the pellets. In this case, 0.075 ml of 37° C. saline solution was added to each pellet and the hydration of the pellet was observed. The third was evaluation of the clot that was formed after wetting with 0.075 ml of 37° C. saline solution.

Results:

Pellets containing gelatin ranging from 2.5% to 7.5% were removed easily from the wells. They also had a good texture and wet easily. Pellets made with 10% gelatin were hard and stiff, hot were easy to remove from the wells. These pellets were slow to wet. The pellets that contained 1.875% gelatin or less were fragile and were difficult to remove from the wells. These pellets all wet rapidly.

Evaluation of the clots formed showed that clots containing 7.5% or 10% gelatin were liquidy and not well formed. The clots formed from gelatin ranging from 2.5 to 3.75% were good and had a small amount of liquid. The remaining pellets did not form good clots and contained a large amount of liquid.

Gelatin was then formulated to 5% and 1.875% in water and held at 37° C. ERL fibrinogen was formulated in CFB and adjusted to a final fibrinogen concentration of 37.5 mg/ml with a pH of 7.4±0.1. The gelatin and fibrinogen solutions were added to the wells of a 96 well flat-bottom plate as follows: row 1: 100 ul of 5% gelatin; row 2: 100 ul of 1.875% gelatin; row 3: 100 ul of 2.5% gelatin; row 4: 100 ul of 0.938% gelatin; row 5: 100 ul of 1.25% gelatin; row 6: 100 ul of 0.469% gelatin. Each well then received 100 ul of fibrinogen solution. The plate was then gently mixed, and placed on ice for 15 minutes. After 15 minutes on ice, the temperature of the gelatin/fibrinogen mixture was 5° C. and the plate was then moved to a −80° C. freezer. After 2 minutes in the −80° C. freezer, the temperature reached −1° C. Each well then received 15 ul of thrombin at 25 U/ml. The plate was then re-frozen and lyophilized.

Four types of evaluation were performed on the pellets. The first involved the ability of the pellets to be removed intact from the well. This was accomplished by inserting a small pipet tip into the center of the well and then attempting to lift out the pellet. The second evaluation was used to determine the wetting ability of the pellets. In this case, 0.075ml of 37° C. saline solution was added to each pellet and the hydration of the pellet was observed. The third was evaluation of the dot that was formed after wetting with 0.075 ml of 37° C. saline solution. The fourth was how much weight the pellet could hold prior to crushing.

Results:

Pellets containing gelatin ranging from 5% to 0.938% were easy to remove from the wells. Pellets containing gelatin at 0.469% were too fragile to remove from the wells. When 75 ul of 37° C. water was added to the pellets, the pellets wet well and good clots were formed for all conditions except for the clot containing 0.469% gelatin.

Pellets were removed from the wells and increasing weights were placed on top of them until the pellets were crushed. A summary of the weight results are shown below in Table 1.

TABLE 1
Results of Weight Required to Crush Pellets Test

| % Gelatin | Weight to Crush (g) |
|---|---|
| 5 | 226 |
| 2.5 | 226 |
| 1.875 | 126 |
| 1.25 | 76 |
| 0.938 | 46 |
| 0.469 | 36 |
| 0 | 26 |

Multiple types of applicators were manufactured by attaching different materials to the ends of 2 ml and 5 ml serological pipettes (with the tapered ends cut off). The materials used included 2 types of PGA BIOFELT® which were cut into discs, as well as a much thicker type of biofelt, Gelfoam®, and a puffed cornstarch material which were all cut into thicker plug shapes. These materials were all attached to the pipette ends by looping a piece of thread through the material on the end of the pipette, and then passing the thread ends back through the pipette. The cotton plug was then inserted to hold the material on the end of the pipette.

ERL fibrinogen was formulated in CFB and adjusted to a final fibrinogen concentration of 37.5 mg/ml with a pH of 7.4±0.1. Two additional fibrinogen formulations were also prepared using different fibrinogen sources (either reconstituted fibrinogen from Kedrion or fibrinogen purified in-house from F1 paste). For the Kedrion fibrinogen formulations, additional sucrose was also added. Recombinant thrombin (RECOTHROM®) was reconstituted with the supplied diluent (0.9% sodium chloride) according to the manufacturer's instructions to a concentration of 1000 units/ml with a pH of 6.0±0.1. A portion of this thrombin solution was also diluted in CTB and adjusted to a final thrombin concentration of 0.1 units/mg of fibrinogen or 25 units/ml thrombin, with a final pH of 7.4±0.1. Once prepared, the final fibrinogen and thrombin solutions were placed on ice and cooled to 4° C.±2° C.

Applicators of each type were then prepared with either a mixture of thrombin and fibrinogen or with thrombin alone. For the mixed thrombin and fibrinogen groups, applicators were manufactured under the following conditions: ERL fibrinogen at 15 mg/cm², fibrinogen purified from F1 paste at 13 mg/cm², Kedrion fibrinogen at 15 mg/cm², and Kedrion fibrinogen at 8 mg/cm². For all of these applicators, the 25 units/ml thrombin solution (at 4° C.±2° c.) was added to round-bottom polypropylene tubes (for a final thrombin concentration of 0.1 units/mg of fibrinogen), followed by the appropriate fibrinogen solution (at 4° C.±2° C.) and briefly mixed. For the thrombin alone condition, the 1000 units/ml thrombin solution (at 4° C.±2° C.) was added to round-bottom polypropylene tubes. The tip of an applicator was then inserted into each tube and allowed to absorb the thrombin or fibrinogen and thrombin solutions for 5 seconds. The tubes were then immediately immersed in liquid nitrogen and frozen. After freezing, the applicators were all placed at −80° C. for at least two hours before being lyophilized in the freeze-dryer.

### Example 27

Multiple types of applicators were manufactured by attaching different materials to the ends of 5 ml serological pipettes (with the tapered ends cut off). The materials used included PGA BIOFELT® which was cut into discs, as well as Gelfoam® and a puffed cornstarch material which were both cut into thicker plug shapes. These materials were all attached to the pipette ends by looping a piece of thread through the material on the end of the pipette, and then passing the thread ends back through the pipette. The cotton plug was then inserted to hold the material on the end of the pipette.

ERL fibrinogen was formulated in CFB and adjusted to a final fibrinogen concentration of 37.5 mg/ml with a pH of 7.4±0.1. A yellow dye was then added to the fibrinogen solution. Recombinant thrombin (RECOTHROM®) was reconstituted with the supplied diluent (0.9% sodium chloride) according to the manufacturer's instructions to a concentration of 1000 units/ml with a pH of 6.0±0.1. A portion of this thrombin solution was also diluted in CTB and adjusted to a final thrombin concentration of 0.1 units/mg of fibrinogen or 25 units/ml thrombin, with a final pH of 7.4±0.1. A blue dye was added to both thrombin solutions. Once prepared, the final fibrinogen and thrombin solutions were placed on ice and cooled to 4° C.±2° C.

Applicators of each type were then prepared with either a mixture of thrombin and fibrinogen or with thrombin alone. For the mixed thrombin and fibrinogen group, 0.043 ml of the 25 units/ml thrombin solution (at 4° C.±2° C.) was added to each round-bottom polypropylene tube, followed by 0.27 ml of the fibrinogen solution (at 4° C.±2° C.). The tubes were then briefly tapped to fully mix the two solutions, which appeared green upon mixing. For the thrombin alone condition, 0.313 ml of the 1000 units/ml thrombin solution (at 4° C.±2° C.) was added to each round-bottom polypropylene tube. The tip of an applicator was then inserted into each tube and allowed to absorb the thrombin or fibrinogen and thrombin solutions for 15 seconds. The tubes were then immediately frozen by immersion in a dry ice/ethanol mixture for 2 minutes. After freezing, the applicators were all placed at −80° C. for at least two hours before being lyophilized in the freeze-dryer.

A splenic injury was created by excising a portion of the spleen with a biopsy punch, deep enough to produce mild to moderate bleeding. Initial bleeding was assessed as mild to moderate or pulsatile for approximately 30 seconds. Shed blood was suctioned from the peritoneal cavity, and a 5 ml applicator was applied with manual pressure to the injured surface of the spleen for 3 minutes. After 30 seconds, the thread was released so that the biofelt disc could remain on the injury site while the pipette was pulled away. The results of this evaluation are presented below in Table 1.

TABLE 1
Summary Data for Mild to Moderate Splenic Injuries: Serological Pipette
Applicators 6 Jan. 2012

| | | Injury Size, | Bleeding | Bleeding | | Actives | |
|---|---|---|---|---|---|---|---|
| Animal # | Injury # | Method | Severity | Description | Applicator | Formulation | Hemostasis? |
| 1 | 7 | 2 mm | Mild to | Oozing, | 5 mL | FAST with | Yes |
| | | Biopsy | Moderate | Flowing | Pipette, | Zymo | |
| | | Punch | | | String/Biofelt | Thrombin | |
| 1 | 8 | 3 mm | Mild to | Oozing, | 5 mL | FAST with | Yes |
| | | Biopsy | Moderate | Flowing | Pipette, | Zymo | |
| | | Punch | | | String/Biofelt | Thrombin | |
| 1 | 9 | 3 mm | Moderate | Flowing | 5 mL | FAST with | No, oozing |
| | | Biopsy | | | Pipette, | Zymo | |
| | | Punch | | | String/Puffed | Thrombin | |
| | | | | | Cornstrach | | |

What is claimed is:

1. A device for treating wounded internal tissue in a mammal comprising:
(a) an applicator; and
(b) a haemostatically effective amount of a haemostatic material.

2. The device according to claim 1, wherein said applicator comprises a rod, plunger or tamper.

3. The device according to claim 2, wherein said applicator further comprises a sheath.

4. The device according to claim 3, wherein substantially all of said haemostatic material is contained within said sheath.

5. The device according to claim 2, wherein said haemostatic material is affixed to a terminal portion of said rod, plunger or tamper.

6. The device according to claim 5, wherein said device further comprises a retainer to maintain said haemostatic material on said terminal portion of said rod, plunger or tamper.

7. The device according to claim 6, wherein said retainer comprises at least one of a hook, tie, clamp, quill, hook and loop fastener, break away layer and claw.

8. The device according to claim 1, wherein said applicator comprises a guide wire.

9. The device according to claim 1, wherein said haemostatic material includes at least one support layer.

10. The device according to claim 9, further comprising at least one physiologically acceptable adhesive between said haemostatic material and said support layer.

11. The device according to claim 1, wherein said haemostatic material also contains one or more of the following: at least one filler; at least one solubilizing agent; at least one foaming agent; and at least one release agent.

12. The device according to claim 1, wherein said haemostatic material also contains at least one therapeutic supplement selected from the group consisting of antibiotics, anticoagulants, steroids, cardiovascular drugs, growth factors, polyclonal antibodies, monoclonal antibodies, chemoattractants, anesthetics, antiproliferatives, antitumor agents, antivirals, cytokines, colony stimulating factors, antifungals, antiparasitics, antiinflammatories, antiseptics, hormones, vitamins, glycoproteins, fibronectin, peptides, proteins, carbohydrates, proteoglycans, antiangiogenins, antigens, nucleotides, lipids, liposomes, fibrinolysis inhibitors, procoagulants, anticoagulants, vascular constrictors and gene therapy reagents.

13. The device according to claim 1, wherein said haemostatic material has been subjected to at least one process selected from the group consisting of freezing, lyophilization, drying, spray-drying, vacuum drying and vitrification, and combinations of two or more thereof.

14. The device according to claim 1, wherein said haemostatic material is cast or formed from a single aqueous solution containing both said fibrinogen component and said fibrinogen activator.

15. The device according to claim 1, wherein said haemostatic material is formed from separate aqueous solutions of said fibrinogen component and said fibrinogen activator.

16. The device according to claim 1, wherein said haemostatic material is substantially homogenous.

17. The device according to claim 5, wherein said applicator includes a grip at the terminal portion of said rod, plunger or tamper opposite said haemostatic material.

18. The device according to claim 1, wherein said applicator includes a buffer or cushion to reduce pressure on said wounded internal tissue during application of said haemostatic material.

19. The device according to claim 1, wherein said applicator comprises a pressure-sensitive mechanism or material.

20. The device according to claim 1, wherein said applicator comprises a pressure-limiting mechanism or material.

21. The device according to claim 1, wherein said applicator comprises a mechanism to facilitate observation of the treatment site.

22. The device of claim 1, wherein said hemostatic material consists essentially of a fibrinogen component and a fibrinogen activator.

23. The device according to claim 22, wherein said haemostatic material comprises at least one layer of said fibrinogen component and at least one layer of said fibrinogen activator.

24. The device according to claim 22, wherein said haemostatic material comprises a plurality of particles consisting essentially of said fibrinogen component and a plurality of particles consisting essentially of said fibrinogen activator.

25. The device according to claim 24, wherein said plurality of particles consisting essentially of said fibrinogen component and said plurality of particles consisting essentially of said fibrinogen activator are admixed.

26. The device of claim 1, wherein said hemostatic material consists essentially of a fibrinogen component.

27. The device of claim 1, wherein said hemostatic material consists essentially of a fibrinogen activator.
